(19) United States
(12) Patent Application Publication
(10) Pub. No.: US 2025/0064938 A1
(43) Pub. Date: Feb. 27, 2025

(54) IONIZABLE LIPIDS FOR MULTIPLE ORGAN TARGETING

(71) Applicant: Massachusetts Institute of Technology, Cambridge, MA (US)

(72) Inventors: Daniel Griffith Anderson, Framingham, MA (US); Bowen Li, Malden, MA (US); Akiva Gordon, Somerville, MA (US); Idris Raji, Boston, MA (US)

(73) Assignee: Massachusetts Institute of Technology, Cambridge, MA (US)

(21) Appl. No.: 18/725,289

(22) PCT Filed: Jan. 3, 2023
(86) PCT No.: PCT/US2023/010027

§ 371 Date: Jun. 28, 2024

Related U.S. Application Data
(60) Provisional application No. 63/296,291, filed on Jan. 4, 2022.

(51) Int. Cl. A61K 47/54; A61K 9/127; A61K 31/7105; A61K 48/00; C07C 233/47; C07D 211/14; C07D 231/12; C07D 233/61; C07D 243/08; C07D 295/13; C12N 15/113

(52) U.S. Cl. CPC A61K 47/543; A61K 9/1272; A61K 31/7105; A61K 47/545; A61K 47/548; A61K 47/554; A61K 48/0033; C07C 233/47; C07D 211/14; C07D 231/12; C07D 233/61; C07D 243/08; C07D 295/13; C12N 15/113; C12N 2310/11; C12N 2310/14; C12N 2310/141; C12N 2310/531

(57) ABSTRACT

Provided herein are lipid compounds of Formulae (I) and (II), and pharmaceutically acceptable salts, co-crystals, tautomers, stereoisomers, solvates, hydrates, polymorphs, isotopically labeled derivatives, prodrugs, and compositions thereof. Also provided are methods and kits involving the inventive lipid compounds, compositions, or formulations for treating and/or preventing diseases (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject, methods for synthesizing the compounds described herein, and compounds described herein synthesized by the synthetic methods described herein. The compounds are effective carriers for the delivery of an agent such as a polynucleotide (e.g., RNA) to a tissue or cell in a subject (e.g., a liver, lung, or spleen tissue/cell).

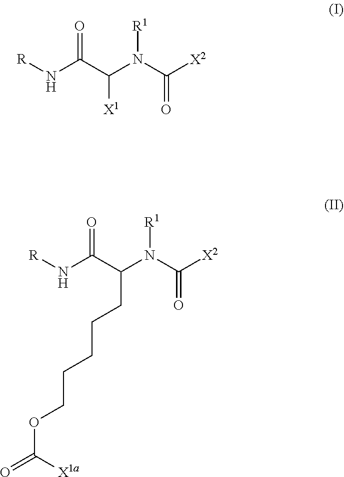

### CROSS-REFERENCE TO RELATED APPLICATIONS

This application claims the benefit of and priority under 35 U.S.C. § 119(e) to U.S. Provisional Application No. 63/296,291, filed Jan. 4, 2022, titled IONIZABLE LIPIDS FOR MULTIPLE ORGAN TARGETING, the contents of which are incorporated herewith by reference in their entirety.

### BACKGROUND OF THE INVENTION

There is considerable interest in developing nucleic acid-based therapeutics. Nucleic acid-based therapeutics in the form of mRNA can supply the body with encoded information for making specific antigen/protein of interest that may provide therapeutic value in connection with specific diseases and are being vigorously pursued in developing vaccines for cancer and other infectious diseases, with other applications including immunotolerance, gene delivery, editing and silencing. Despite their potentials, the clinical applications of mRNA and siRNA have been limited by the inability to site-specifically deliver them to target organs and tissues. Their large size, multiple negative charges, and susceptibility to endonuclease degradation make their clinical translation even more challenging. As naked mRNA is inherently unstable and prone to rapid nuclease degradation, an appropriate delivery vehicle is important for RNA therapy. This necessitates the need to develop efficient delivery systems that can preserve the integrity of mRNA/siRNA and induce organ/tissue-selective delivery.

Among the many delivery systems that have been studied, lipid nanoparticles (LNPs) have emerged as the platform of choice for delivery of mRNA. Typically, lipid nanoparticles consist of: i) a helper lipid; ii) a PEG-lipid; iii) cholesterol or a sterol; and iv) an ionizable lipid. In 2018, LNPs enabled the first FDA approval of an siRNA drug (Onpattro); two years later, two SARS-CoV-2 vaccines (Comirnaty, Spikevax) based on LNPs containing mRNA were developed during the COVID-19 pandemic. The ionizable lipid is a particularly important component, as it is responsible for complexing mRNA and effecting organ/tissue-selective delivery of such mRNA. Once LNPs are internalized by cells via endocytosis, acidification of the endosomal compartment leads to protonation of the ionizable lipid, destabilizing both the LNP and endosomal membranes, and ultimately enabling the RNA payload to escape from the endosome and enter the cytosol. Structurally, ionizable lipids comprise amine head group, linker and hydrocarbon tails. Unsaturation and branching in tails, as well as pKa of amine head groups have been identified to improve the efficiency of delivery of mRNA.¹ Likewise, biodegradability in the form of ester bonds have become an important feature to help mitigate toxicity to target tissues.⁸ The space of possible structures for each component is vast and provides unique properties and utility for LNP designs.

Each of the three FDA-approved RNA-LNPs mentioned above carry a unique ionizable lipid. For example, Onpattro contains the ionizable lipid DLin-MC3-DMA (MC3), a terminal tertiary amine linked to symmetrical linoleic tails. However, the low biodegradability of this lipid limits clearance and raises toxicity concerns, limiting both the therapeutic window and dosage repeatability. The LNP used by Pfizer's mRNA vaccine (Comirnaty) adopts an ionizable lipid with a terminal amino alcohol headgroup linked to two identical, biodegradable lipid tails. Similarly, the LNP in Spikevax utilizes an ionizable lipid with a shorter amino alcohol headgroup and distinct biodegradable lipid tails. The role ionizable lipids play in LNPs has also encouraged novel lipid designs that diverge from traditional patterns.

Thus, given the targetability, adaptability, and biocompatability of lipids for delivering agents including mRNA, it is important to develop lipid compounds for delivering mRNA therapeutics in vivo to treat various diseases (e.g., proliferative diseases, infectious diseases) and activate the immune system. It is important to develop a fast and efficient method to quickly generate a large number of lipids to achieve safe and efficient delivery of agents (e.g., mRNA).

Lipid nanoparticles potently transfect organs such as liver, lungs and spleen, making them suitable for developing therapeutics and vaccines for diseases affecting these organs. The biodegradable tails in the lipids disclosed herein decrease the likelihood of inducing toxicity to target organs.

Despite the need for novel ionizable lipid designs, their development is hindered by cumbersome, time-consuming synthesis and screening methods. Additionally, while rational design strategies can provide meaningful improvements, they dramatically limit the design space possible and will overlook unexpected yet powerful lipid designs. Incorporating a rational design strategy with a high-throughput synthesis strategy can provide a structurally diverse in silico library of hundreds of thousands of lipids; however, physical limitations hinder the synthesis and screening of such an extensive library. Therefore, the use and training of machine learning algorithms (MLA) provide an opportunity to reduce synthesis and screening requirements. A major roadblock to developing an effective computer model is the availability of a sufficiently large data set for training. The Ugi 3-component reaction (3-CR) is suitable for high throughput combinatorial synthesis (HTCS) of ionizable lipids.³ Although 3-CR lipid synthesis was completed without needing toxic catalysts, solvent exchange, or (de)protection steps within 24 hours at room temperature,³ the 3-CR lipids did not have biodegradable tails, did not show organ-specific targeting of lipid nanoparticles, and were not suitable for applications other than vaccines.

### SUMMARY OF THE INVENTION

The present disclosure describes the development of a high throughput synthesis of over 600 ionizable lipids and screening for their ability to deliver mRNA locally and to specific organs in mice. Biodegradable lipids were identified that are efficient at delivering mRNA to the liver, lungs and spleen.

In one aspect, the present disclosure provides compounds of Formula (I):

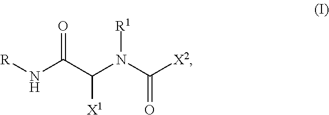

and pharmaceutically acceptable salts, co-crystals, tautomers, stereoisomers, solvates, hydrates, polymorphs, and isotopically labeled derivatives thereof, wherein R, R¹, X¹, and X² are as defined herein.

In another aspect, the present disclosure provides compounds of Formula (II):

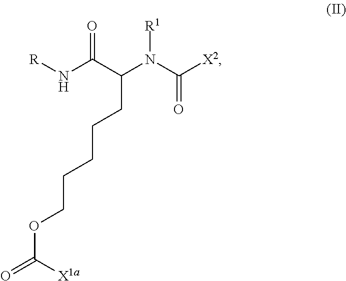

and pharmaceutically acceptable salts, co-crystals, tautomers, stereoisomers, solvates, hydrates, polymorphs, and isotopically labeled derivatives, wherein R, R¹, X^{1a}, and X² are as defined herein.

In another aspect, provided herein are compositions (e.g., pharmaceutical compositions) including a compound provided herein, and optionally an agent (e.g., a small organic molecule, inorganic molecule, nucleic acid, protein, peptide, or polynucleotide (e.g., RNA)). In certain embodiments, the composition is in the form of a particle (e.g., a nanoparticle or a microparticle). In certain embodiments, a composition described herein includes a therapeutically or prophylactically effective amount of a compound described herein. The compositions may be useful in delivering an agent (e.g., a polynucleotide (e.g., RNA)) to a cell, organ, or tissue, in treating a disease (e.g., a genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject in need thereof, or in preventing a disease in a subject in need thereof. In certain embodiments, the compound is administered or used in treating and/or preventing a disease (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject in need thereof.

In still another aspect, described herein are kits including a container with a compound or composition described herein. A kit described herein may include a single dose or multiple doses of the compound or composition. The described kits may be useful in treating and/or preventing a disease (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject in need thereof, and/or in delivering an agent (e.g., a polynucleotide (e.g., RNA)) to a cell, tissue, or organ in a subject. In certain embodiments, a kit described herein further includes instructions for using the compound or composition included in the kit.

In certain embodiments, the compositions are useful in delivering an agent (e.g., a polynucleotide (e.g., RNA)) to a cell, tissue, or organ in a subject. In certain embodiments, the compositions are adjuvants in mRNA vaccine systems. In certain embodiments, the compositions are used to deliver mRNA agents as part of vaccine systems with a range of antigens including tumor-associated antigens, personalized multi-epitope antigens, and bacterial and viral proteins. In certain embodiments, the compositions activate an innate immune response in the subject. Another aspect of the present disclosure relates to methods of treating a disease in a subject in need thereof, the methods comprising administering to the subject a therapeutically effective amount of a compound or composition described herein. In another aspect, the present disclosure provides methods of preventing a disease in a subject in need thereof, the methods comprise administering to the subject a prophylactically effective amount of a compound or composition described herein. In another aspect, provided herein are methods of delivering an agent (e.g., RNA) to a cell, tissue, or organ (e.g., a lung, liver, or spleen cell/tissue) in a subject.

In yet another aspect, the present disclosure provides methods for making compounds of Formula (I). In yet another aspect, the present disclosure provides compounds of Formula (I), synthesized by methods described herein.

In yet another aspect, the present disclosure provides compounds and compositions described herein for use in a method of the disclosure (e.g., a method of delivering an agent (e.g., a polynucleotide (e.g., RNA)) to a subject, tissue, or cell (e.g., a tissue or cell of the liver, lung or spleen), or a method of treating and/or preventing a disease (e.g., proliferative disease, infectious disease, autoimmune disease)).

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The term “aliphatic” includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, the term “alkyl” includes straight, branched and cyclic alkyl groups. An analogous convention applies to other generic terms such as “alkenyl”, “alkynyl”, and the like. Furthermore, the terms “alkyl”, “alkenyl”, “alkynyl”, and the like encompass both substituted and unsubstituted groups. In certain embodiments, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the disclosure contain 1-20 aliphatic carbon atoms. In certain other embodiments, the alkyl, alkenyl, and alkynyl groups employed in the disclosure contain 1-10 aliphatic carbon atoms. In yet other embodiments, the alkyl, alkenyl, and alkynyl groups employed in the disclosure contain 1-8 aliphatic carbon atoms. In still other embodiments, the alkyl, alkenyl, and alkynyl groups employed in the disclosure contain 1-6 aliphatic carbon atoms. In yet other embodiments, the alkyl, alkenyl, and alkynyl groups employed in the disclosure contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₅ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₈ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

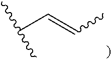

may be an (E)- or (Z)-double bond.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃-s cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiiranyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

“Heteroaryl” refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to an optionally substituted alkyl group substituted by an optionally substituted heteroaryl group.

“Unsaturated” or “partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups). Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups, are further referred to using the suffix -ene, e.g., alkylene, alkenylene, alkynylene, carbocyclylene, heterocyclylene, arylene, and heteroarylene.

An atom, moiety, or group described herein may be unsubstituted or substituted, as valency permits, unless otherwise provided expressly. The term “optionally substituted” refers to substituted or unsubstituted.

A group is optionally substituted unless expressly provided otherwise. The term “optionally substituted” refers to being substituted or unsubstituted. In certain embodiments, alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. In certain embodiments, the substituent is a carbon atom substituent. In certain embodiments, the substituent is a nitrogen atom substituent. In certain embodiments, the substituent is an oxygen atom substituent. In certain embodiments, the substituent is a sulfur atom substituent.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R—, —NR_{bb}SO₂R^{aa}, —NR^{bb}C(═O)N(R^{bb})₂, —C(═NR^{bb})R^{aa}, —C(═NR^{bb})ORₐₐ, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, —NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups;
each instance of R^{bb} is, independently, selected from hydrogen, —OH, —OR^{aa}, —N(R^{cc})₂, —CN, —C(═O)Rₐₐ, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ee})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

“Acyl” refers to a moiety of the formula: —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb}SO₂R^{aa}, —C(═S)N(R^{bb})₂, —C(═O)SR^{aa}, or —C(═S)SR^{aa}, wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{aa})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R⁾₃⁺X⁻, —P(OR^{cc})₂, —P(OR⁾₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂ wherein R^{aa}, R^{bb}, and R^{cc} are as defined herein).

A “hydrocarbon chain” refers to a substituted or unsubstituted divalent alkyl, alkenyl, or alkynyl group. A hydrocarbon chain includes (1) one or more chains of carbon atoms immediately between the two radicals of the hydrocarbon chain; (2) optionally one or more hydrogen atoms on the chain(s) of carbon atoms; and (3) optionally one or more substituents (“non-chain substituents,” which are not hydrogen) on the chain(s) of carbon atoms. A chain of carbon atoms consists of consecutively connected carbon atoms (“chain atoms”) and does not include hydrogen atoms or heteroatoms. However, a non-chain substituent of a hydrocarbon chain may include any atoms, including hydrogen atoms, carbon atoms, and heteroatoms. For example, hydrocarbon chain —C^{A}H(C^{B}H₂C^{C}H₃)— includes one chain atom C^{A}, one hydrogen atom on C^{A}, and non-chain substituent —(C^{B}H₂C^{C}H₃). The term “Cₓ hydrocarbon chain,” wherein x is a positive integer, refers to a hydrocarbon chain that includes x number of chain atom(s) between the two radicals of the hydrocarbon chain. If there is more than one possible value of x, the smallest possible value of x is used for the definition of the hydrocarbon chain. For example, —CH(C₂H₅)— is a C₁ hydrocarbon chain, and

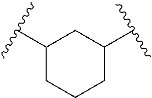 [SMILES: *C1CCCC(*)C1]

is a C₃ hydrocarbon chain. When a range of values is used, the meaning of the range is as described herein. For example, a C₃₋₁₀ hydrocarbon chain refers to a hydrocarbon chain where the number of chain atoms of the shortest chain of carbon atoms immediately between the two radicals of the hydrocarbon chain is 3, 4, 5, 6, 7, 8, 9, or 10. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH—(CH₂)₂—, —CH₂—C≡C—CH₂—, and —C≡C—CH═CH— are all examples of a unsubstituted and unsaturated hydrocarbon chain. In certain embodiments, the hydrocarbon chain is unsubstituted (e.g., —C≡C— or —(CH₂)₄—). In certain embodiments, the hydrocarbon chain is substituted (e.g., —CH(C₂H₅)— and —CF₂—). Any two substituents on the hydrocarbon chain may be joined to form an optionally substituted carbocyclyl, optionally substituted heterocyclyl, optionally substituted aryl, or optionally substituted heteroaryl ring. For instance,

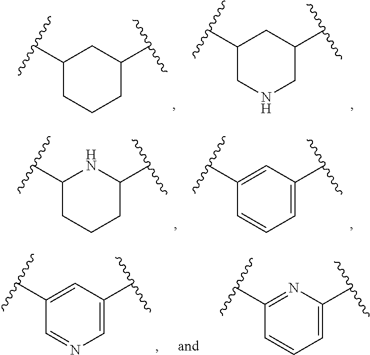 [SMILES: *C1CCCC(*)C1 ; *C1CNCC(*)C1 ; *C1CCCC(*)N1 ; *c1cccc(*)c1 ; *c1cncc(*)c1 ; *c1cccc(*)n1]

are all examples of a hydrocarbon chain. In contrast, in certain embodiments,

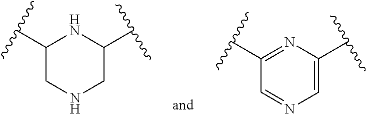 [SMILES: *C1CNCC(*)N1 ; *c1cncc(*)n1]

are not within the scope of the hydrocarbon chains described herein. When a chain atom of a Cₓ hydrocarbon chain is replaced with a heteroatom, the resulting group is referred to as a Cₓ hydrocarbon chain wherein a chain atom is replaced with a heteroatom, as opposed to a Cₓ₋₁ hydrocarbon chain. For example,

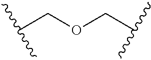 [SMILES: *COC*]

is a C₃ hydrocarbon chain wherein one chain atom is replaced with an oxygen atom.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and claims. The invention is not intended to be limited in any manner by the above exemplary listing of substituents.

### Other Definitions

The following definitions are more general terms used throughout the present application.

The term “solvate” refers to forms of the compound, or a salt thereof that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

As used herein the term “inhibit” or “inhibition” in the context of disease treatment (e.g., tumor growth), refers to a reduction in the level of tumor growth. In some embodiments, the term refers to a reduction of the level of tumor growth to a level that is statistically significantly lower than an initial level, which may, for example, be a baseline level of tumor growth. In some embodiments, the term refers to a reduction of the level of tumor growth to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of tumor growth.

When a compound, pharmaceutical composition, method, use, or kit is referred to as “selectively,” “specifically,” or “competitively” targeting a disease in a target (e.g., cell (e.g., cancer cell)), the compound, pharmaceutical composition, method, use, or kit inhibits the target, to a greater extent (e.g., not less than 2-fold, not less than 5-fold, not less than 10-fold, not less than 30-fold, not less than 100-fold, not less than 1,000-fold, or not less than 10,000-fold; and/or: not more than 2-fold, not more than 5-fold, not more than 10-fold, not more than 30-fold, not more than 100-fold, not more than 1,000-fold, or not more than 10,000-fold) than binding or inhibiting a different target (e.g., different cell).

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. A “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a biological sample.

The term “tissue” refers to any biological tissue of a subject (including a group of cells, a body part, or an organ) or a part thereof, including blood and/or lymph vessels, which is the object to which a compound, particle, and/or composition of the invention is delivered.

A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for treating a disease.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for preventing and/or treating a disease (e.g., a proliferative disease (e.g., cancer)).

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MD); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

The term “spleen disease” refers to a disease of the spleen. Example of spleen diseases include, but are not limited to, splenomegaly, spleen cancer, asplenia, spleen trauma, idiopathic purpura, Felty's syndrome, Hodgkin's disease, and immune-mediated destruction of the spleen.

The term “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawl symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D.C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

An “effective amount” of a compound or composition described herein refers to an amount sufficient to elicit the desired biological response. An effective amount of a polymer or composition described herein may vary depending on such factors as the desired biological endpoint, the pharmacokinetics of the composition, the condition being treated, the mode of administration, and the age and health of the subject. In certain embodiments, an effective amount is a therapeutically effective amount. In certain embodiments, an effective amount is a prophylactically effective amount. In certain embodiments, an effective amount is the amount of a composition or pharmaceutical composition described herein in a single dose. In certain embodiments, an effective amount is the combined amounts of a composition or pharmaceutical composition described herein in multiple doses.

A “therapeutically effective amount” of a compound or composition described herein is an amount sufficient to provide a therapeutic benefit in the treatment of a condition or to delay or minimize one or more symptoms associated with the condition. A therapeutically effective amount of a compound or composition means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound or composition described herein is an amount sufficient to prevent a condition, or one or more symptoms associated with the condition or prevent its recurrence. A prophylactically effective amount of a compound or composition means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The terms “nucleic acid” or “nucleic acid sequence”, “nucleic acid molecule”, “nucleic acid fragment” or “polynucleotide” are used interchangeably. A polynucleotide molecule is a biopolymer composed of nucleotide monomers covalently bonded in a chain. DNA (deoxyribonucleic acid) and RNA (ribonucleic acid) are examples of polynucleotides with distinct biological function. DNA consists of two chains of polynucleotides, with each chain in the form of a helical spiral. RNA is more often found in nature as a single-strand folded onto itself. Exemplary types of RNA include double-stranded RNA (dsRNA), small interfering RNA (siRNA), short hairpin (shRNA), microRNA (miRNA), messenger RNA (mRNA), antisense RNA, transfer RNA (tRNA), small nuclear RNA (snRNA), and ribosomal RNA (rRNA).

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region.

The term “RNA interference” or “RNAi” refers to a biological process in which RNA molecules inhibit gene expression or translation, by neutralizing targets mRNA molecules. Since the discovery of RNAi and its regulatory potentials, it has become evident that RNAi has immense potential in suppression of desired genes. RNAi is now known as precise, efficient, stable, and better than antisense technology for gene suppression. Two types of small ribonucleic acids molecules are central to RNA interference: miRNA and siRNA. These small RNAs can bind to mRNA molecules and either increase or decrease their activity (e.g., preventing an mRNA from being translated into a protein). The RNAi pathway is found in many eukaryotes, including animals, and is initiated by the enzyme Dicer, which cleaves long dsRNA molecules into short double-stranded fragments of ˜20 nucleotide siRNAs. Each siRNA is unwound into two single-stranded RNAs (ssRNAs), the passenger strand and the guide strand. The passenger strand is degraded and the guide strand is incorporated into the RNA-induced silencing complex (RISC). The most well-studied outcome is post-transcriptional gene silencing, which occurs when the guide strand pairs with a complementary sequence in a mRNA molecule and induces cleavage by Argonaute 2 (Ago2), the catalytic component of the RISC complex. In some organisms, this process spreads systematically, despite the initially limited molar concentrations of siRNA.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

The disclosure is not intended to be limited in any manner by the above exemplary listing of substituents. Additional terms may be defined in other sections of this disclosure.

### BRIEF DESCRIPTION OF THE DRAWINGS

FIGS. 1A-1B show UGI 4-component reaction for high-throughput synthesis of ionizable lipids. FIG. 1A shows the reactants, amine, isocyanide, aldehyde, and carboxylic acid are classified as headgroup, linker, lipid tail 1, and lipid tail 2 respectively as depicted in the scheme. FIG. 1B shows the synthesis of a representative lipid using UGI 4-CR.

FIG. 2 shows high-throughput synthesis and screening of ionizable lipid library. FIG. 2A shows a schematic illustration of lipid library synthesis and screening to generate data for training ML algorithm. FIG. 2B shows all four components (reactants) used for synthesis of 384 ionizable lipid library. FIG. 2C shows a heatmap showing in-vitro transfection data in HeLa cells after treatment with LNP encapsulating mLuc made from 384 ionizable lipids. FIGS. 2D-2I show batch-based screening of 384 LNP encapsulating mLuc. FIG. 2D shows IVIS imaging of luciferase expression 6 hours after IM injection of LNP batches containing Tails 1; FIG. 2E shows a graph comparing levels of luciferase within LNP batches containing Tails 1; FIG. 2F shows IVIS imaging of luciferase expression 6 hours after IM injection of LNP batches containing Tails 2 and top performing Tails 1; FIG. 2G shows a graph comparing levels of luciferase within LNP batches containing Tails 2 and top performing Tails 1; FIG. 2H shows IVIS imaging of luciferase expression 6 hours after IM injection of LNP batches containing Linkers and top performing Tails 1 and 2; FIG. 2I shows a graph comparing levels of luciferase within LNP batches containing Linkers and top performing Tails 1 and 2. FIG. 2J shows the top 8 combinations of Tails 1, Tails 2, Linkers were reacted with 25 amines to give 200 ionizable lipids. FIG. 2K shows a heatmap showing in-vitro transfection data in HeLa cells after treatment with LNP made from 200 ionizable lipids. Lipid tail/linker combinations are shown on the Y-axis and are of formula WXY, wherein W refers to Tail 1 aldehyde number shown in FIG. 4A, X refers to Tail 2 carboxylic acid number shown in FIG. 4A, and Y refers to the Linker isocyanide number shown in FIG. 4A. For example, “111” refers to aldehyde 1 for Tail 1, carboxylic acid 1 for Tail 2, and isocyanide 1 for the Linker. HG1-25 on the X-axis refer to amine Headgroups 1-25 shown in FIG. 4A. FIG. 2L shows in-vitro transfection of HeLa cells by lipid nanoparticles made from 200 lipids.

FIGS. 3A-3H show the ML algorithm was developed using data from LNP screen. FIG. 3A shows a schematic illustration of processes involved in design of machine learning algorithm. FIGS. 3B-3D show receiver operating characteristic area under the curve (ROC or ROC-AUC) and precision-recall area under the curve (PR or PR-AUC) curves evaluating model performance for: XGBoost (FIG. 3B); Logistic regression (FIG. 3C); random forest (FIG. 3D). FIGS. 3E-3H show top 10 contributing descriptors in: Amine headgroups (FIG. 3E); Linkers (FIG. 3F); Tails 1 (FIG. 3G); and Tails 2 (FIG. 3H), calculated from XGBoost model. The specified molecular descriptors are AATSC8i (Average centered Broto-Moreau autocorrelation—lag 8/weighted by first ionization potential), AATSC8v (Average centered Broto-Moreau autocorrelation—lag 8/weighted by van der Waals volumes), ATSC8v (Centered Broto-Moreau autocorrelation—lag 8/weighted by van der Waals volumes), MLFER_E (Excessive molar refraction), GATS4c (Geary autocorrelation—lag 4/weighted by charges), AATS6i (Average Broto-Moreau autocorrelation—lag 6/weighted by first ionization potential), AATS3e (Average Broto-Moreau autocorrelation—lag 3/weighted by Sanderson electronegativities), JGI3 (Mean topological charge index of order 3), BCUTw.11 (nhigh lowest atom weighted BCUTS), ATSC5i (Centered Broto-Moreau autocorrelation—lag 5/weighted by first ionization potential), nAtomLAC (Number of atoms in the longest aliphatic chain), nO (Number of oxygen atoms), VE3_Dt (Logarithmic coefficient sum of the last eigenvector from detour matrix), AMR (Molar refractivity), VE1_Dt (Coefficient sum of the last eigenvector from detour matrix), A Log P (Ghose-Crippen Log Kow), ATS6m (Broto-Moreau autocorrelation—lag 6/weighted by mass), GATS5c (Geary autocorrelation—lag 5/weighted by charges), AATSC3i (Average centered Broto-Moreau autocorrelation—lag 3/weighted by first ionization potential), BCUTp.1h (nlow highest polarizability weighted BCUTS), nBondsD (Number of double bonds), ATSC6c (Centered Broto-Moreau autocorrelation—lag 6/weighted by charges), ATSC7c (Centered Broto-Moreau autocorrelation—lag 7/weighted by charges), AATSC6m (Average centered Broto-Moreau autocorrelation—lag 6/weighted by mass), ATSC3c (Centered Broto-Moreau autocorrelation—lag 3/weighted by charges), ATSC1c (Centered Broto-Moreau autocorrelation—lag 1/weighted by charges), ATSC8c (Centered Broto-Moreau autocorrelation—lag 8/weighted by charges), AATS0p (Average Broto-Moreau autocorrelation—lag 0/weighted by polarizabilities), VR1_Dt (Randic-like eigenvector-based index from detour matrix), AATS1p (Average Broto-Moreau autocorrelation—lag 1/weighted by polarizabilities), ATSC3v (Centered Broto-Moreau autocorrelation—lag 3/weighted by van der Waals volumes), nH (Number of hydrogen atoms), ATSC6c (Centered Broto-Moreau autocorrelation—lag 6/weighted by charges), ATSC2m (Centered Broto-Moreau autocorrelation—lag 2/weighted by mass), apol (Sum of the atomic polarizabilities (including implicit hydrogens)), AATS0e (Average Broto-Moreau autocorrelation—lag 0/weighted by Sanderson electronegativities), and AATS5p (Average Broto-Moreau autocorrelation—lag 5/weighted by polarizabilities).

FIGS. 4A-4K show the screening of an expanded library with ML yields 119-23, a multi-purpose ionizable lipid. FIG. 4A shows an expanded library containing 40,000 (40 amines×10 linkers x 10 Tail 1×10 Tail 2) ionizable lipids was proposed which were then screened using the ML algorithm. FIG. 4B shows pie charts showing ML algorithm rankings of top 10 amines, linkers, tails 1, and tails 2. FIG. 4C shows that, to test the ML algorithm prediction, a library of 16 ionizable lipids was made from top two amines, linkers, tails 1, and tails 2. FIG. 4D shows IVIS imaging of luciferase expression 6 hours after IM injection of LNP encapsulating mLuc made from top 16 ionizable lipids. FIG. 4E shows the structure of highest performing ionizable lipid 119-23. FIG. 4F shows a comparison of liver and spleen transfection activity of LNP made from C12-200, MC3, and 119-23 FIG. 4G shows SORT was used to navigate LNP to the lungs, showing 119-23 performed better than C12-200 and MC3. FIG. 4H shows a schematic illustration of Ail4 mice model. FIGS. 4I-4K show tdTomato⁺ cells after intravenous injection of LNP made from 119-23 containing 0.75 mg/kg CRE mRNA in the liver (ANOVA with Tukey post test, * indicates significance vs. control, FIG. 4I), spleen (ANOVA with Tukey post test, * indicates significance vs. control, FIG. 4J), and lung (separate t tests for each cell type, FIG. 4K).

FIG. 5 shows in vivo activity of lipid nanoparticles made from 200 compound lipid library in HeLa cells.

FIG. 6 shows resynthesized hits from in-vivo and in-vitro final screen.

FIG. 7 shows in-vivo evaluation of mLuc encapsulating lipid nanoparticles made from resynthesized top performing lipids. On the left is bio-distribution data in various organs after IV administration, while the images on the right show data from IM administration of lipid nanoparticles.

FIG. 8A shows additional linkers, and FIG. 8B shows additional lipid products.

FIG. 9 shows the structures of lipids that were formulated into lipid nanoparticles encapsulating mLuc and screened in vivo by IV administration.

FIGS. 10A-10C show the comparison of performance of the 119-23 and 242-23 LNP's with MC3. FIG. 10A shows both 119-23 and 242-23 LNPs potently transfect the liver and performed better than MC3 LNP. FIG. 10B shows SORT of LNPs to the lungs revealed both 119-23 and 242-23 performed better than MC3 in lungs transfection. FIG. 10C shows the time course comparison of erythropoietin protein production by 119-23 and MC3 LNP encapsulating Epo mRNA after intravenous injection. 119-23 shows higher EPO transfection compared with MC3.

FIG. 11 shows structural analogs of lipid 119-23 made by systematically varying the tail length.

FIG. 12 shows in vivo transfection activity of tail length variants of 119-23.

FIG. 13 shows tdTomato⁺ cells after intravenous injection of LNP containing 0.75 mg/kg CRE mRNA in the liver (ANOVA with Tukey post test, * indicates significance vs. control).

FIG. 14 shows tdTomato⁺ cells after intravenous injection of LNP containing 0.75 mg/kg CRE mRNA in the spleen (ANOVA with Tukey post test).

### DETAILED DESCRIPTION OF CERTAIN EMBODIMENTS OF THE INVENTION

Before the disclosed systems, compounds, compositions, methods, reagents, uses, and kits are described in more detail, it should be understood that the aspects described herein are not limited to specific embodiments, methods, apparati, or configurations, and as such can, of course, vary. It is also to be understood that the terminology used herein is for the purpose of describing particular aspects only and, unless specifically defined herein, is not intended to be limiting.

Provided herein are new ionizable lipid compounds, compositions, kits, methods for the treatment of various diseases (e.g., genetic diseases, proliferative diseases, hematological diseases, neurological diseases, painful condition, psychiatric disorder, metabolic disorder, long-term medical condition, inflammatory diseases, autoinflammatory diseases, liver diseases, lung diseases, spleen diseases, familial amyloid neuropathy, cardiovascular diseases, viral infection, infectious diseases, fibrotic condition, or autoimmune diseases).

Also provided are methods of synthesizing compounds described herein. Additionally provided are methods of delivering agents (e.g., RNA (e.g., mRNA)) to cells for the treatment of various diseases (e.g., genetic diseases, proliferative diseases, hematological diseases, neurological diseases, painful condition, psychiatric disorder, metabolic disorder, long-term medical condition, inflammatory diseases, autoinflammatory diseases, liver diseases, lung diseases, spleen diseases, familial amyloid neuropathy, cardiovascular diseases, viral infection, infectious diseases, fibrotic condition, or autoimmune diseases).

In one aspect, disclosed are compounds of Formula (I):

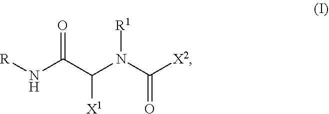

or a pharmaceutically acceptable salt, co-crystal, tautomer, stereoisomer, solvate, hydrate, polymorph, or isotopically enriched derivative thereof, wherein:
R is substituted or unsubstituted alkyl, substituted or unsubstituted heteroalkyl, or substituted or unsubstituted carbocyclyl;
R¹ is substituted or unsubstituted alkyl, substituted or unsubstituted heteroalkyl, or substituted or unsubstituted heterocyclyl; and
X¹ and X² are each independently substituted or unsubstituted C₈-C₁₀₀ aliphatic, or substituted or unsubstituted C₈-C₁₀₀ heteroaliphatic.
   Formula (I) includes substituent X¹. In certain embodiments, X¹ is substituted or unsubstituted C₁₂-C₃₀ heteroaliphatic. In certain embodiments, X¹ is substituted or unsubstituted C₁₂-C₂₄ heteroaliphatic. In certain embodiments, X¹ is of the formula:

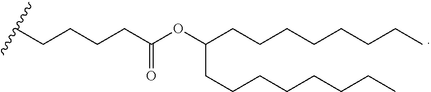 [SMILES: *CCCCC(=O)OC(CCCCCCCC)CCCCCCCC]

In certain embodiments, X¹ is of the formula:

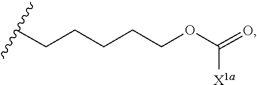

wherein X^{1a} is substituted or unsubstituted C₈-C₁₀₀ aliphatic, or substituted or unsubstituted C₈-C₁₀₀ heteroaliphatic. In certain embodiments, X¹ is of the formula:

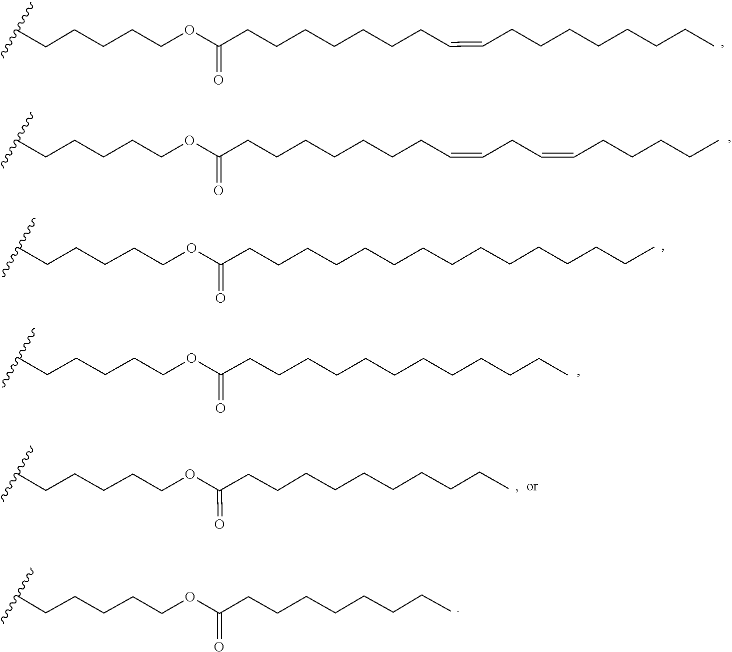 [SMILES: *CCCCCOC(=O)CCCCCCC/C=C\CCCCCCCC ; *CCCCCOC(=O)CCCCCCC/C=C\C/C=C\CCCCC ; *CCCCCOC(=O)CCCCCCCCCCCCCCC ; *CCCCCOC(=O)CCCCCCCCCCCC ; *CCCCCOC(=O)CCCCCCCCCC ; *CCCCCOC(=O)CCCCCCCC]

In another aspect, the compounds of Formula (I) is of Formula (II):

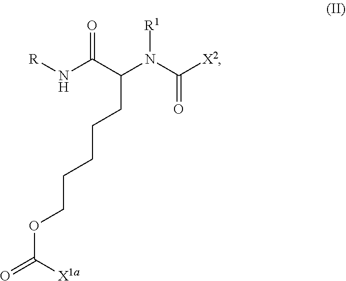

or a pharmaceutically acceptable salt, co-crystal, tautomer, stereoisomer, solvate, hydrate, polymorph, or isotopically enriched derivative thereof, wherein:
R is substituted or unsubstituted alkyl, substituted or unsubstituted heteroalkyl, or substituted or unsubstituted carbocyclyl;
R¹ is substituted or unsubstituted alkyl, substituted or unsubstituted heteroalkyl, or substituted or unsubstituted heterocyclyl;
X² is substituted or unsubstituted C₈-C₁₀₀ aliphatic, or substituted or unsubstituted C₈-C₁₀₀ heteroaliphatic; and
X^{1a} is substituted or unsubstituted C₈-C₁₀₀ aliphatic, or substituted or unsubstituted C₈-C₁₀₀ heteroaliphatic.

Formula (I) and Formula (II) includes the substituent R. In certain embodiments, R is substituted or unsubstituted alkyl. In certain embodiments, R is unsubstituted alkyl. In certain embodiments, R is substituted or unsubstituted C₁-C₆ alkyl. In certain embodiments, R is substituted or unsubstituted C₁-C₆ alkyl. In certain embodiments, R is substituted or unsubstituted C₂-C₄ alkyl. In certain embodiments, R is of the formula:

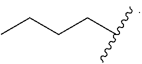 [SMILES: *CCCC]

In certain embodiments R is of the formula:

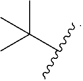 [SMILES: *C(C)(C)C]

In certain embodiments, R is of the formula:

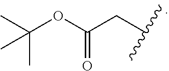 [SMILES: *CC(=O)OC(C)(C)C]

In certain embodiments, R is of the formula:

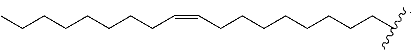 [SMILES: *CCCCCCCC/C=C\CCCCCCCC]

In certain embodiments, R is of the formula:

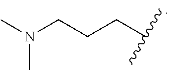 [SMILES: *CCCN(C)C]

In certain embodiments, R is of the formula:

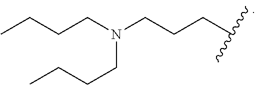 [SMILES: *CCCN(CCCC)CCCC]

In certain embodiments, R is of the formula:

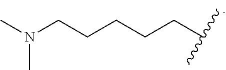 [SMILES: *CCCCCN(C)C]

In certain embodiments, R is of the formula:

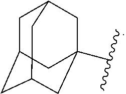 [SMILES: *C12CC3CC(CC(C3)C1)C2]

In certain embodiments, R is of the formula:

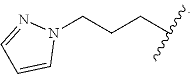 [SMILES: *CCCn1cccn1]

In certain embodiments, R is substituted or unsubstituted carbocyclyl. In certain embodiments, R is unsubstituted carbocyclyl. In certain embodiments, R is substituted or unsubstituted cyclohexyl. In certain embodiments, R is unsubstituted cyclohexyl.

Formula (I) and Formula (II) includes the substituent R¹. In certain embodiments, R¹ is substituted or unsubstituted alkyl. In certain embodiments, R¹ is substituted or unsubstituted C₁-C₆ alkyl. In certain embodiments, R¹ is of the formula:

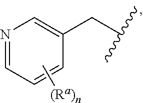

wherein R^{a} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; and n is 1, 2, 3, or 4. In certain embodiments, R¹ is substituted C₂-C₃ alkyl. In certain embodiments, R¹ is of the formula:

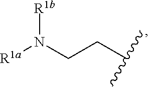

wherein R^{1a} and R^{1b} are each independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or R^{1a} and R^{1b} are joined together to form an optionally substituted heterocyclic ring. In certain embodiments, R^{1a} and R^{1b} are each independently optionally substituted C₁-C₆ alkyl. In certain embodiments, R^{1a} and R^{1b} are substituted or unsubstituted methyl. In certain embodiments, R¹ is of the formula:

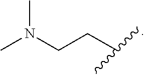 [SMILES: *CCN(C)C]

In certain embodiments, R¹ is of the formula:

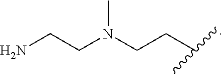 [SMILES: *CCN(C)CCN]

In certain embodiments, R¹ is of the formula:

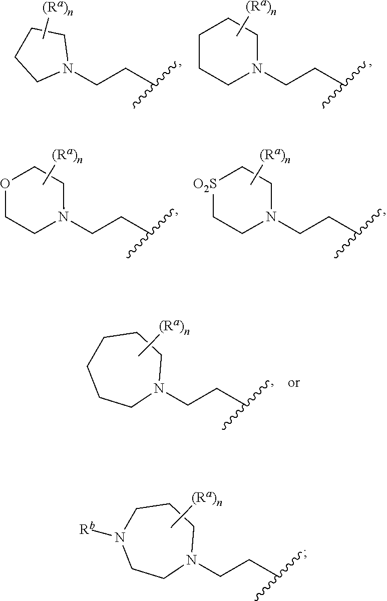

wherein R^{a} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; R^{b} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or a nitrogen protecting group; and n is 1, 2, 3, or 4. In certain embodiments, R¹ is of the formula:

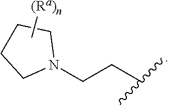

In certain embodiments, R¹ is of the formula:

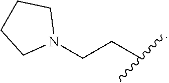 [SMILES: *CCN1CCCC1]

In certain embodiments, R¹ is of the formula:

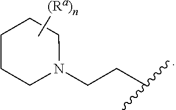

In certain embodiments, R¹ is of the formula:

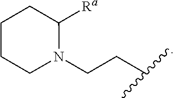

In certain embodiments, R¹ is of the formula:

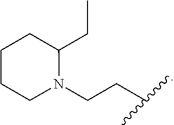 [SMILES: *CCN1CCCCC1CC]

In certain embodiments, R¹ is of the formula:

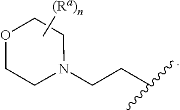

In certain embodiments, R¹ is of the formula:

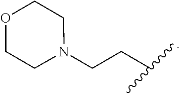 [SMILES: *CCN1CCOCC1]

In certain embodiments, R¹ is of the formula:

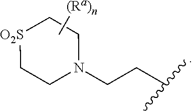

In certain embodiments, R¹ is of the formula:

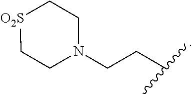 [SMILES: *CCN1CCS(=O)(=O)CC1]

In certain embodiments, R¹ is of the formula:

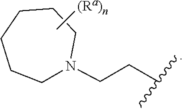

In certain embodiments, R¹ is of the formula:

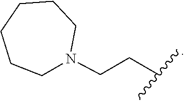 [SMILES: *CCN1CCCCCC1]

In certain embodiments, R¹ is of the formula:

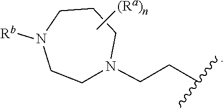

In certain embodiments, R¹ is of the formula:

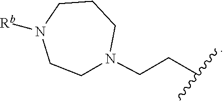

In certain embodiments, R¹ is of the formula:

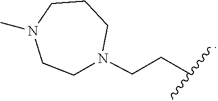 [SMILES: *CCN1CCCN(C)CC1]

In certain embodiments, R¹ is of the formula:

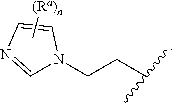

In certain embodiments, R¹ is of the formula:

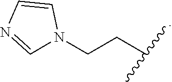 [SMILES: *CCn1ccnc1]

In certain embodiments R¹ is of the formula:

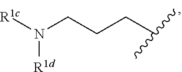

wherein R^{1c} and R^{1d} are each independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or R^{1a} and R^{1b} are joined together to form an optionally substituted heterocyclic ring. In certain embodiments, R^{1c} and R^{1d} are each independently optionally substituted C₁-C₆ alkyl. In certain embodiments, R^{1c} and R^{1d} are substituted or unsubstituted methyl. In certain embodiments, R¹ is of the formula:

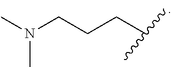 [SMILES: *CCCN(C)C]

In certain embodiments, R¹ is of the formula:

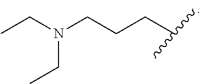 [SMILES: *CCCN(CC)CC]

In certain embodiments, R¹ is of the formula:

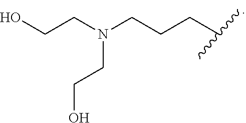 [SMILES: *CCCN(CCO)CCO]

In certain embodiments, R¹ is of the formula:

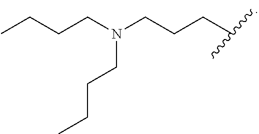 [SMILES: *CCCN(CCCC)CCCC]

In certain embodiments, R^{1c} and R^{1d} are joined together to form an optionally substituted heterocyclic ring. In certain embodiments, R¹ is of the formula:

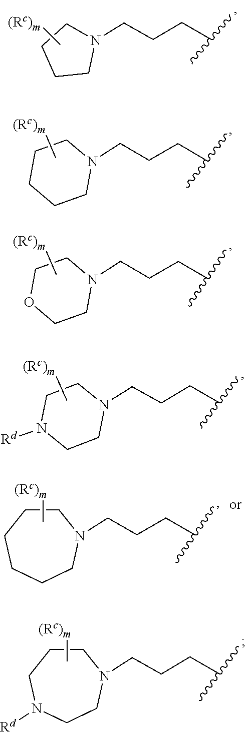

wherein R^{c} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; R^{d} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or a nitrogen protecting group; and m is 1, 2, 3, or 4. In certain embodiments, R¹ is of the formula:

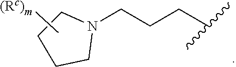

In certain embodiments, R¹ is of the formula:

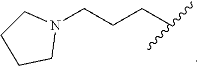 [SMILES: *CCCN1CCCC1]

In certain embodiments, R¹ is of the formula:

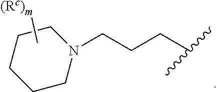

In certain embodiments, R¹ is of the formula:

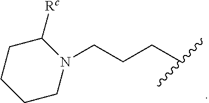

In certain embodiments, R¹ is of the formula:

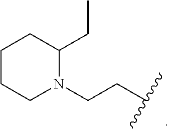 [SMILES: *CCN1CCCCC1CC]

In certain embodiments, R¹ is of the formula:

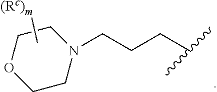

In certain embodiments, R¹ is of the formula:

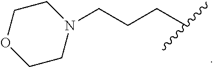 [SMILES: *CCCN1CCOCC1]

In certain embodiments, R¹ is of the formula:

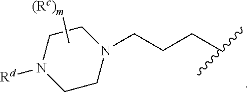

In certain embodiments, R¹ is of the formula:

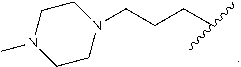 [SMILES: *CCCN1CCN(C)CC1]

In certain embodiments, R¹ is of the formula:

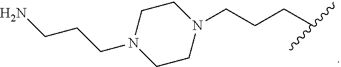 [SMILES: *CCCN1CCN(CCCN)CC1]

In certain embodiments, R¹ is of the formula:

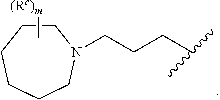

In certain embodiments R¹ is of the formula:

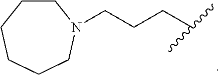 [SMILES: *CCCN1CCCCCC1]

In certain embodiments, R¹ is of the formula:

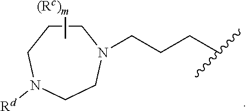

In certain embodiments, R¹ is of the formula:

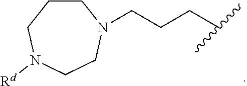

In certain embodiments, R¹ is of the formula:

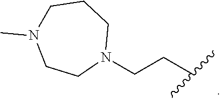 [SMILES: *CCN1CCCN(C)CC1]

In certain embodiments, R¹ is substituted or unsubstituted heterocyclyl.
In certain embodiments, R¹ is of the formula:

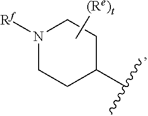

wherein R^{e} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; R^{f} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or a nitrogen protecting group; and t is 1, 2, 3, or 4. In certain embodiments, R¹ is of the formula:

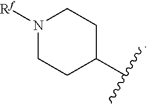

In certain embodiments, R¹ is of the formula:

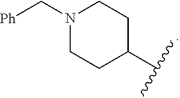 [SMILES: *C1CCN(Cc2ccccc2)CC1]

Formula (I) and Formula (II) includes the substituent X². In certain embodiments, X² is substituted or unsubstituted C₈-C₁₀₀ aliphatic. In certain embodiments, X² is substituted or unsubstituted C₁₂-C₂₀ aliphatic. In certain embodiments, X² is unsubstituted C₈-C₁₀₀ aliphatic. In certain embodiments, X² is substituted or unsubstituted C₁₂-C₂₀ aliphatic. In certain embodiments, X² is of the formula:

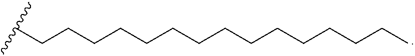 [SMILES: *CCCCCCCCCCCCCCC]

In certain embodiments, X² is of the formula:

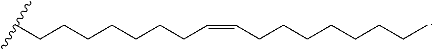 [SMILES: *CCCCCCC/C=C\CCCCCCCC]

In certain embodiments, X² is of the formula:

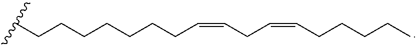 [SMILES: *CCCCCCC/C=C\C/C=C\CCCCC]

In certain embodiments, X² is substituted or unsubstituted C₈-C₁₀₀ heteroaliphatic. In certain embodiments, X² is substituted or unsubstituted C₅-C₂₄ heteroaliphatic. In certain embodiments, X² is of the formula:

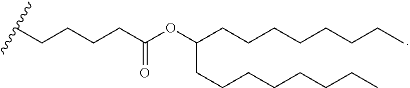 [SMILES: *CCCCC(=O)OC(CCCCCCCC)CCCCCCCC]

In certain embodiments, X² is of the formula:

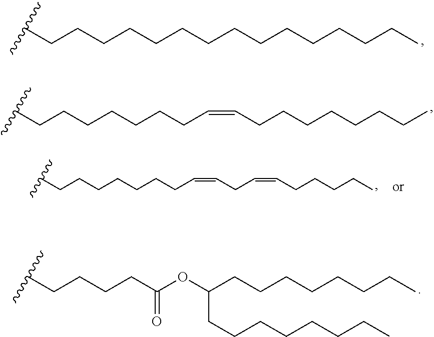 [SMILES: *CCCCCCCCCCCCCCC ; *CCCCCCC/C=C\CCCCCCCC ; *CCCCCCC/C=C\C/C=C\CCCCC ; *CCCCC(=O)OC(CCCCCCCC)CCCCCCCC]

Formula (II) includes the substituent X^{1a}. In certain embodiments, X^{1a} is substituted or unsubstituted C₈-C₁₀₀ aliphatic. In certain embodiments, X^{1a} is substituted or unsubstituted C₈-C₁₈ aliphatic. In certain embodiments, X^{1a} is unsubstituted C₈-C₁₈ aliphatic.

In certain embodiments, the compound of Formula (I) is of the formula:

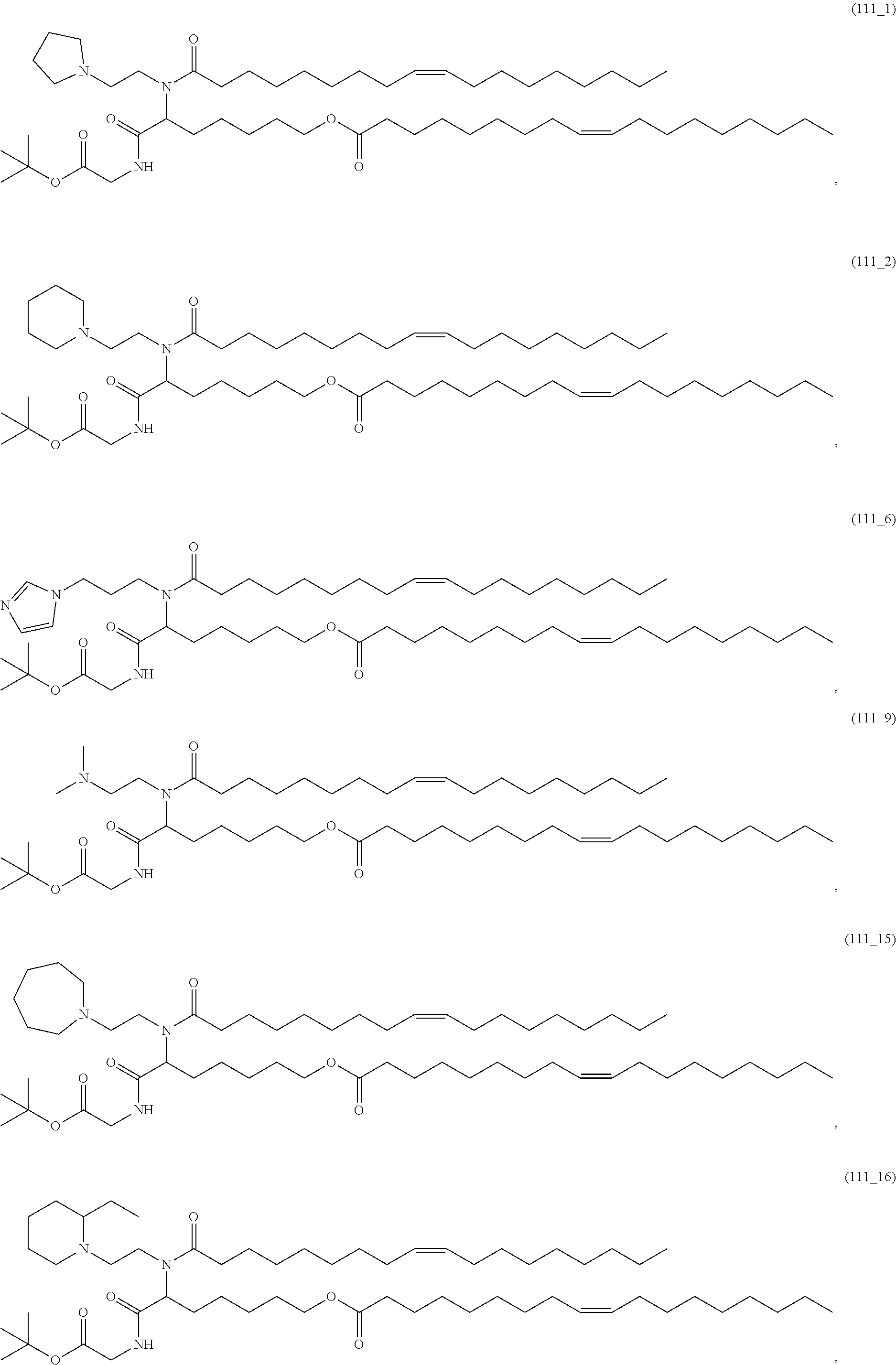

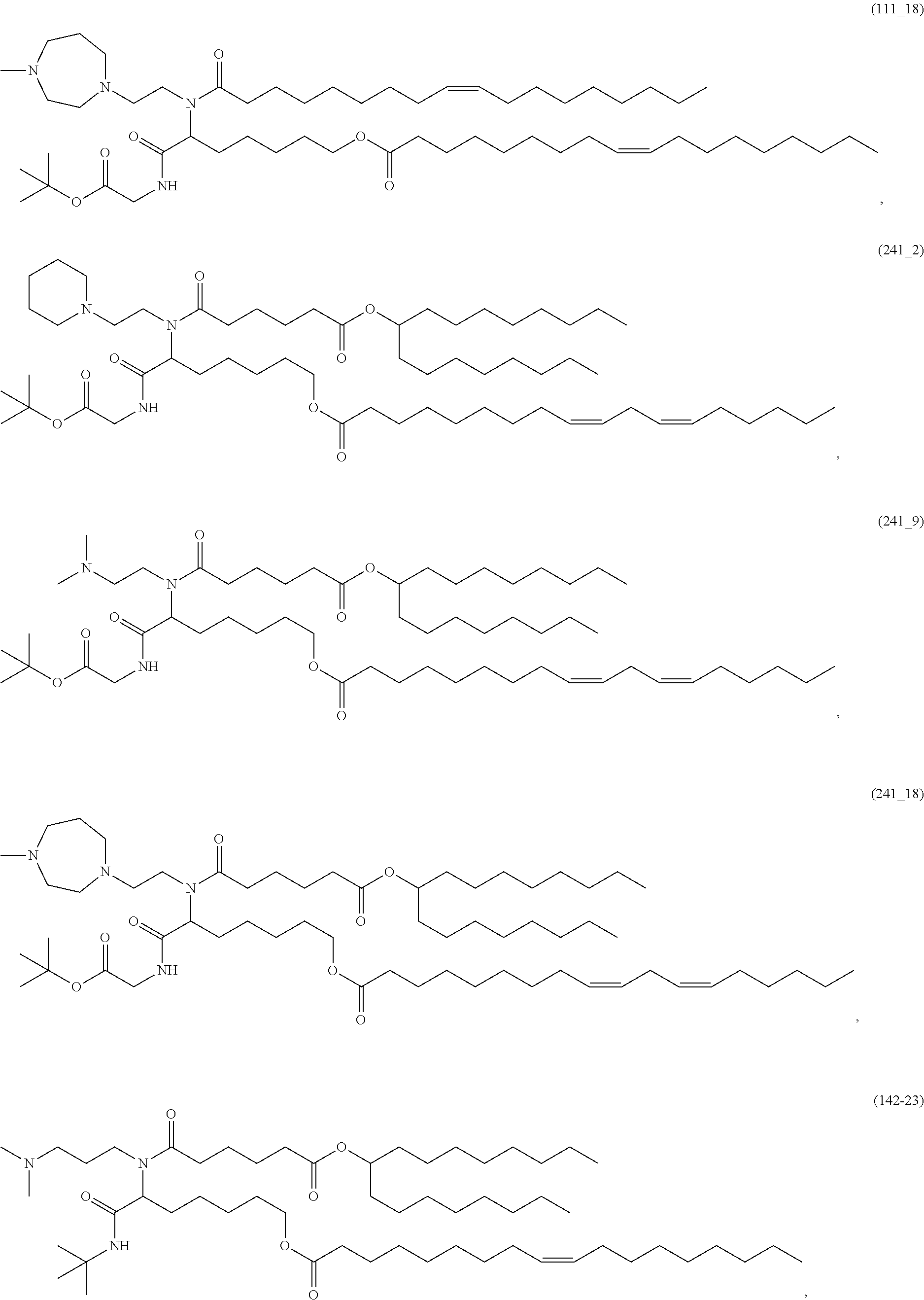

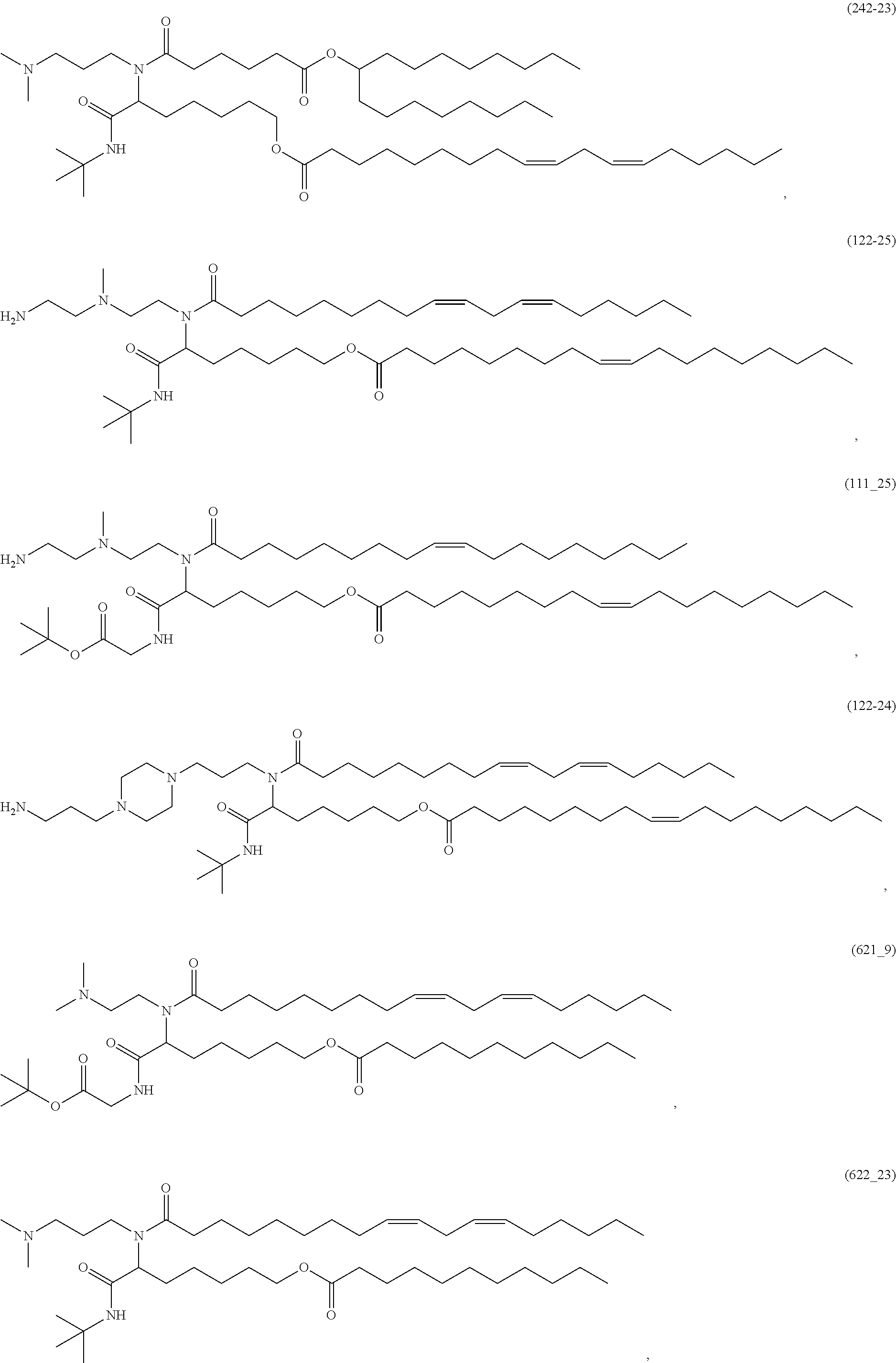

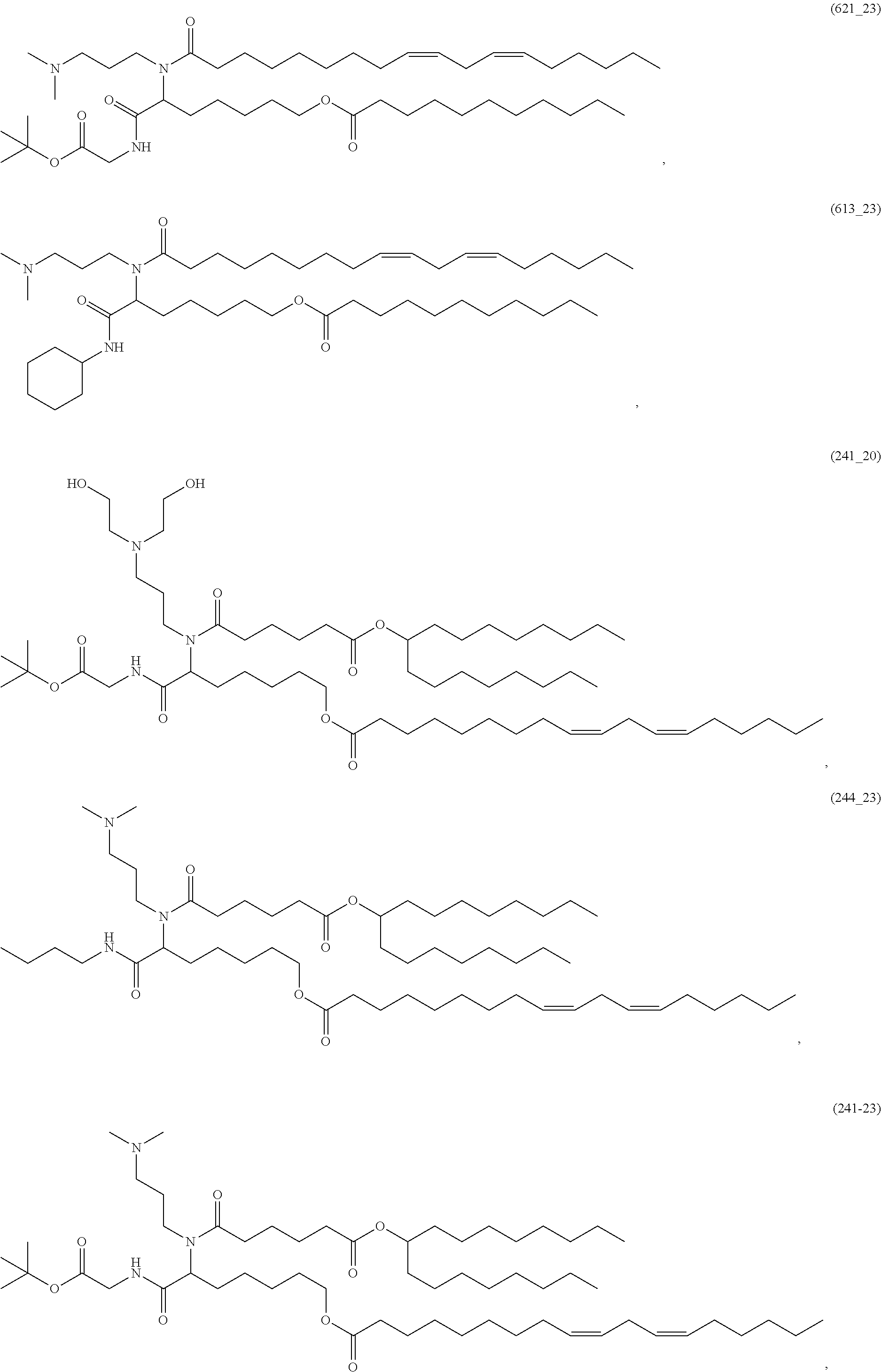

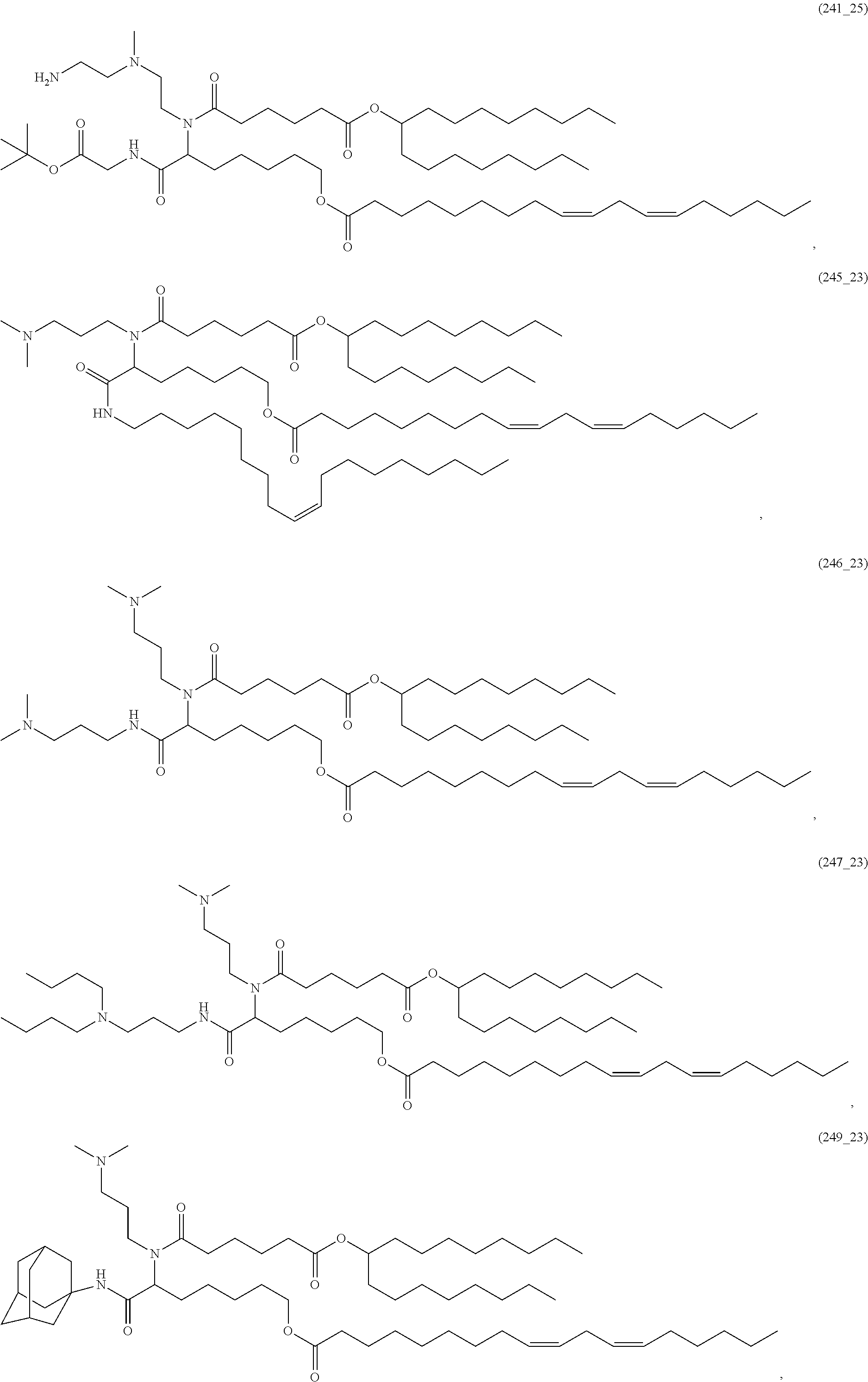

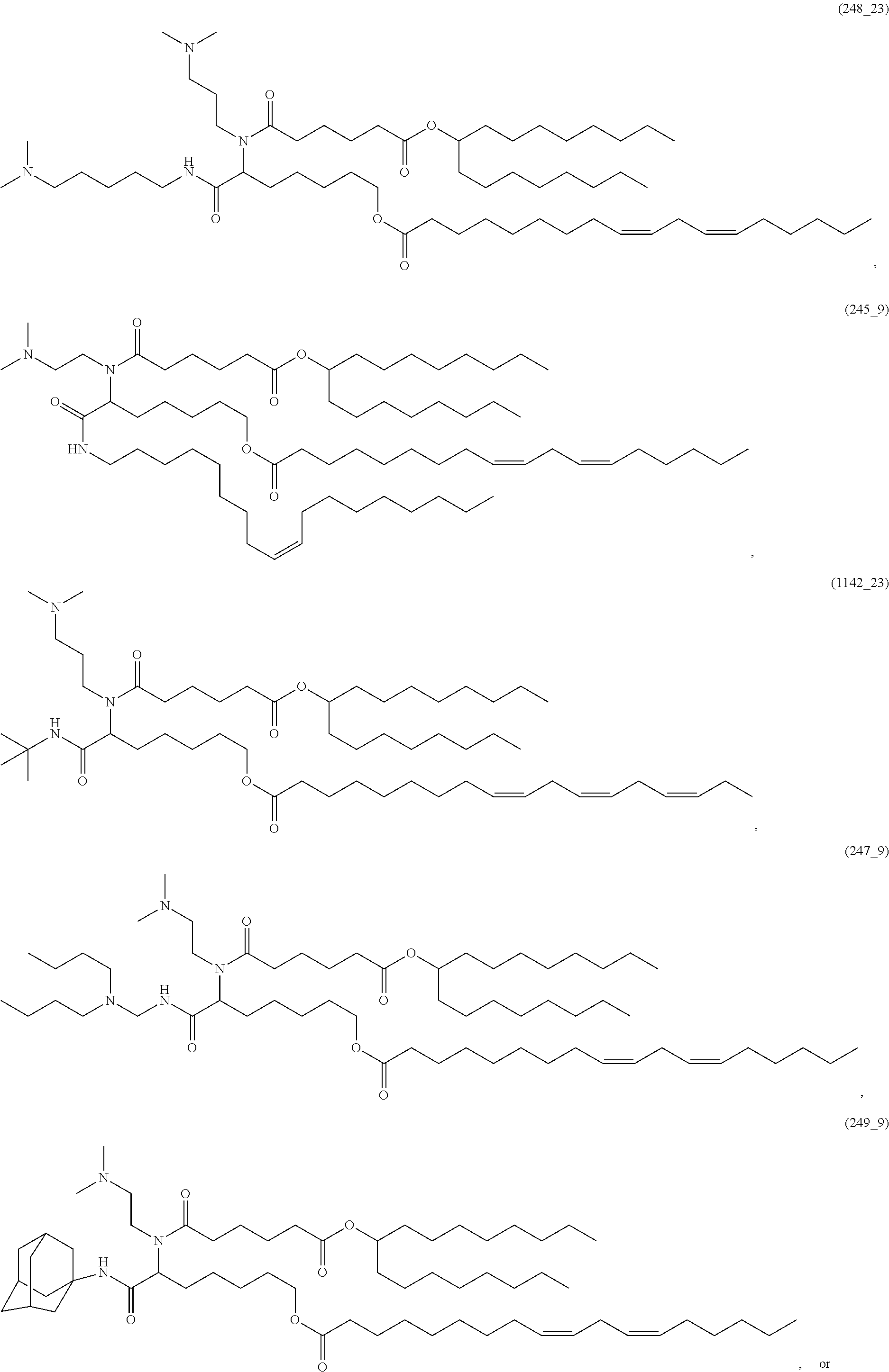

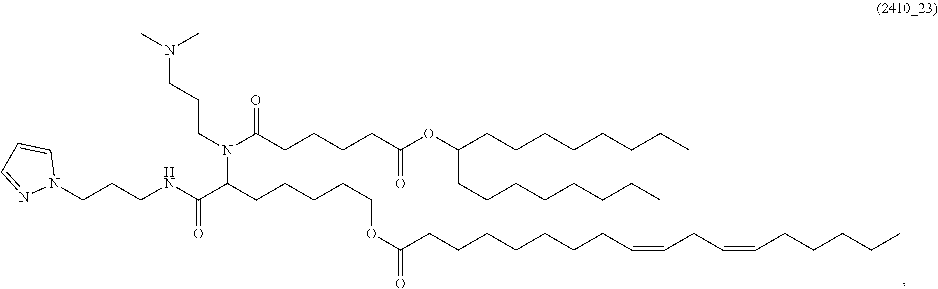 [SMILES: CCCCC/C=C\C/C=C\CCCCCCCC(=O)OCCCCCC(C(=O)NCCCn1cccn1)N(CCCN(C)C)C(=O)CCCCC(=O)OC(CCCCCCCC)CCCCCCCC]

or a pharmaceutically acceptable salt, co-crystal, tautomer, stereoisomer, solvate, hydrate, polymorph, or isotopically enriched derivative thereof.

In certain embodiments, the compound of Formula (I) is 119-23:

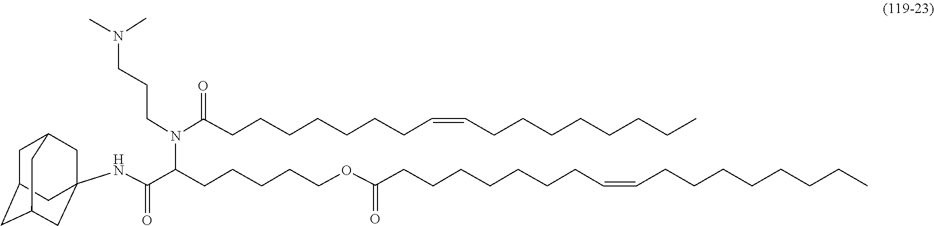 [SMILES: CCCCCCCC/C=C\CCCCCCCC(=O)OCCCCCC(C(=O)NC12CC3CC(CC(C3)C1)C2)N(CCCN(C)C)C(=O)CCCCCCC/C=C\CCCCCCCC]

or a pharmaceutically acceptable salt, co-crystal, tautomer, stereoisomer, solvate, hydrate, polymorph, or isotopically enriched derivative thereof.

In certain embodiments, the compound of Formula (I) is of formula:

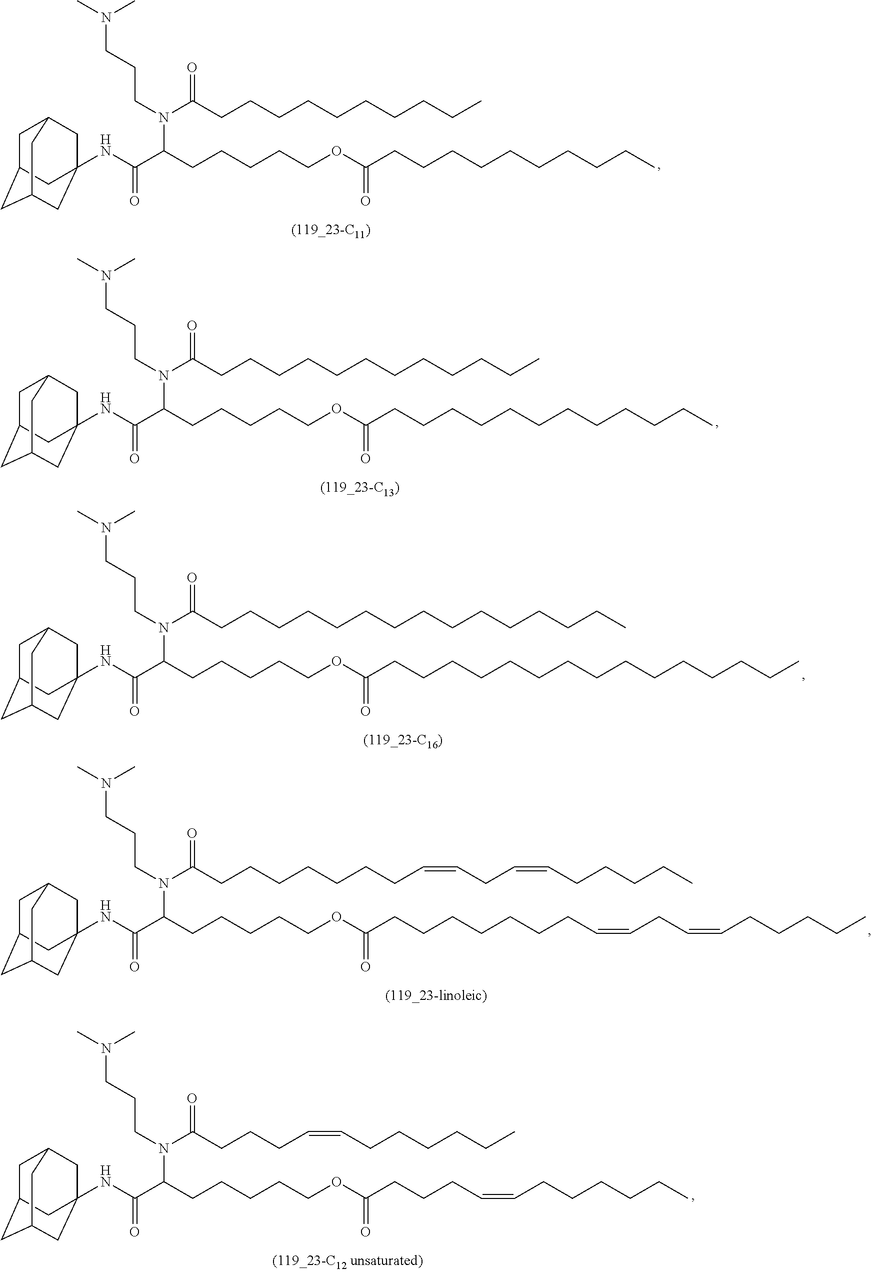 [SMILES: CCCCCCCCCCC(=O)OCCCCCC(C(=O)NC12CC3CC(CC(C3)C1)C2)N(CCCN(C)C)C(=O)CCCCCCCCCC ; CCCCCCCCCCCCC(=O)OCCCCCC(C(=O)NC12CC3CC(CC(C3)C1)C2)N(CCCN(C)C)C(=O)CCCCCCCCCCCC ; CCCCCCCCCCCCCCCC(=O)OCCCCCC(C(=O)NC12CC3CC(CC(C3)C1)C2)N(CCCN(C)C)C(=O)CCCCCCCCCCCCCCC ; CCCCC/C=C\C/C=C\CCCCCCCC(=O)OCCCCCC(C(=O)NC12CC3CC(CC(C3)C1)C2)N(CCCN(C)C)C(=O)CCCCCCC/C=C\C/C=C\CCCCC ; CCCCCC/C=C\CCCC(=O)OCCCCCC(C(=O)NC12CC3CC(CC(C3)C1)C2)N(CCCN(C)C)C(=O)CCC/C=C\CCCCCC]

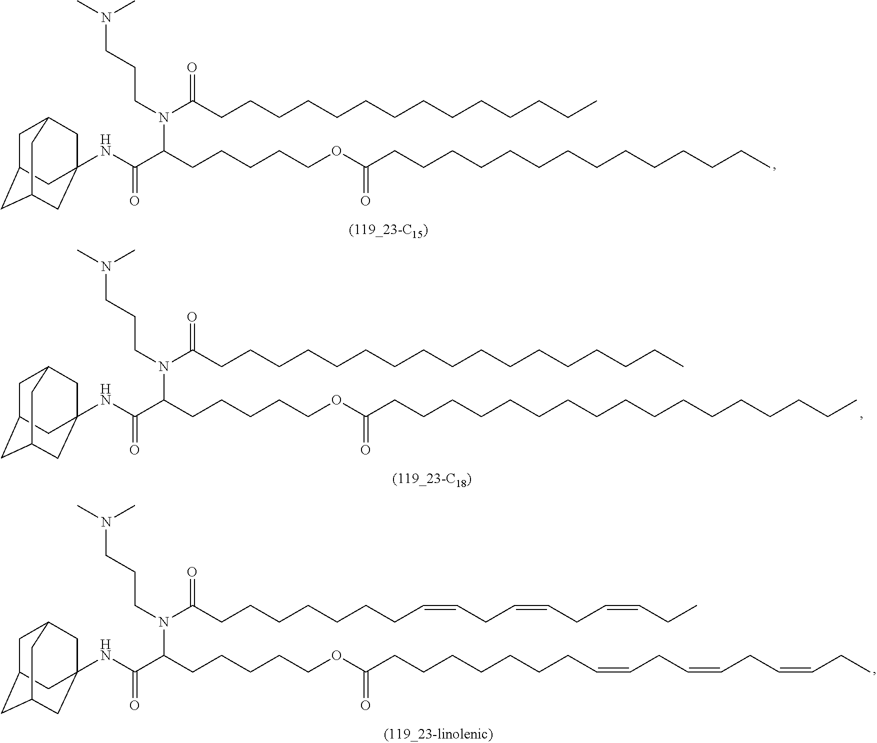

or a pharmaceutically acceptable salt, co-crystal, tautomer, stereoisomer, solvate, hydrate, polymorph, or isotopically enriched derivative thereof.

In certain embodiments, the compound of Formula (I) is 224-23:

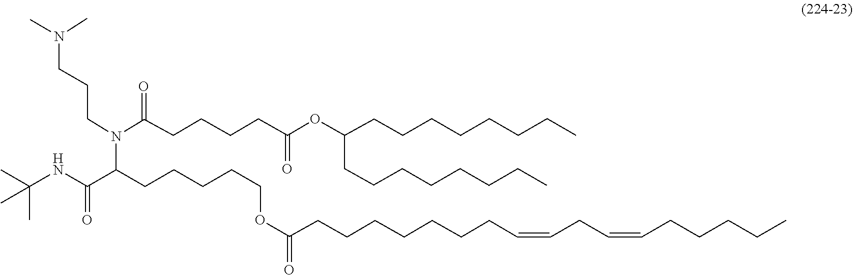 [SMILES: CCCCC/C=C\C/C=C\CCCCCCCC(=O)OCCCCCC(C(=O)NC(C)(C)C)N(CCCN(C)C)C(=O)CCCCC(=O)OC(CCCCCCCC)CCCCCCCC]

or a pharmaceutically acceptable salt, co-crystal, tautomer, stereoisomer, solvate, hydrate, polymorph, or isotopically enriched derivative thereof.

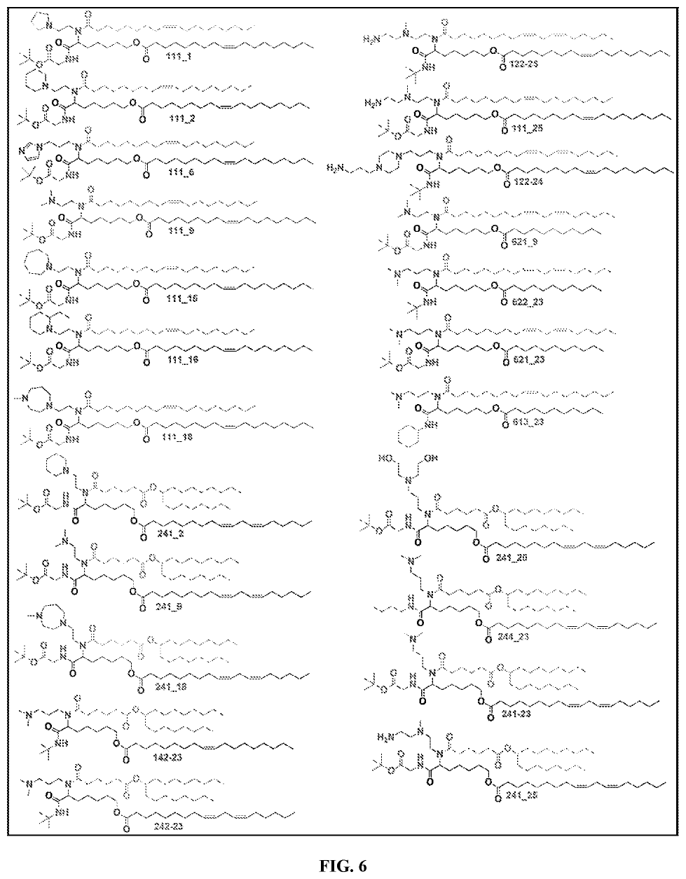

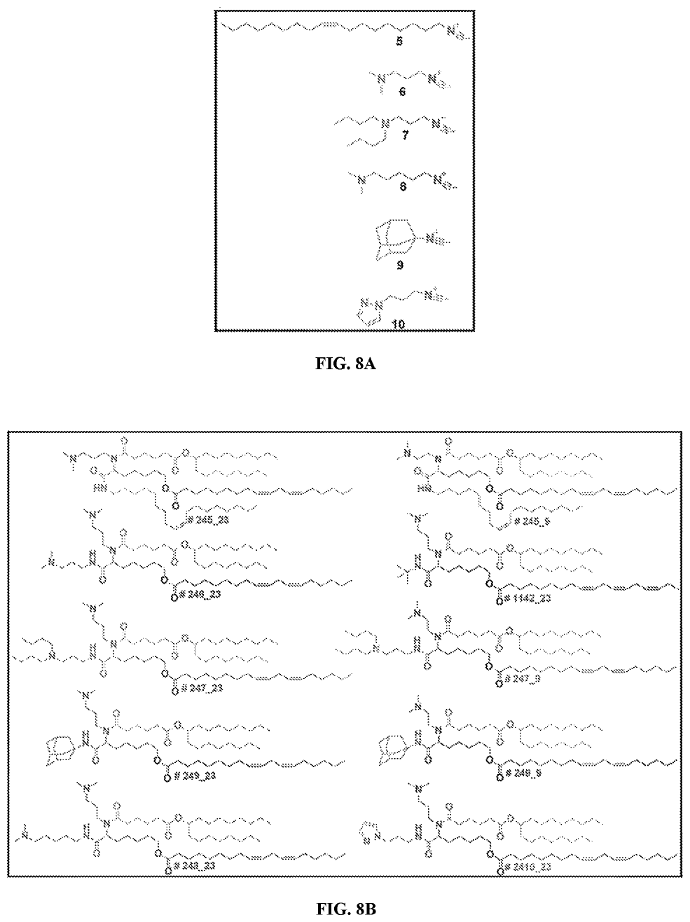

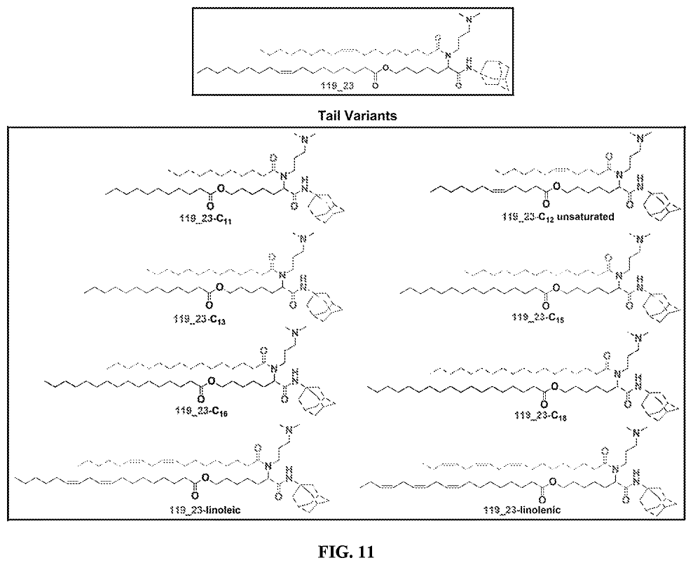

In certain embodiments, the compound of Formula (I) is a compound provided in any one of the Examples below. In certain embodiments, the compound of Formula (I) is a compound provided in Example 1 below. In certain embodiments, the compound of Formula (I) is a compound provided in Example 2 below. In certain embodiments, the compound of Formula (I) is a compound provided in Example 3 below. In certain embodiments, the compound of Formula (I) is a compound provided in Example 4 below. In certain embodiments, the compound of Formula (I) is a compound provided in Example 5 below. In certain embodiments, the compound of Formula (I) is a compound provided in Example 6 below. In certain embodiments, the compound of Formula (I) is a compound provided in any one of the FIGs. In certain embodiments, the compound of Formula (I) is a compound provided in FIG. 6. In certain embodiments, the compound of Formula (I) is a compound provided in FIG. 8B. In certain embodiments, the compound of Formula (I) is a compound provided in FIG. 11.

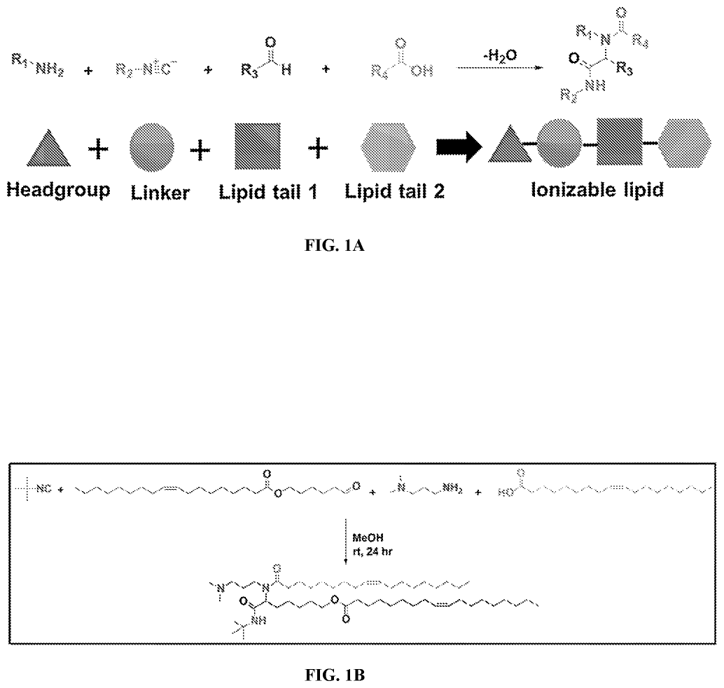

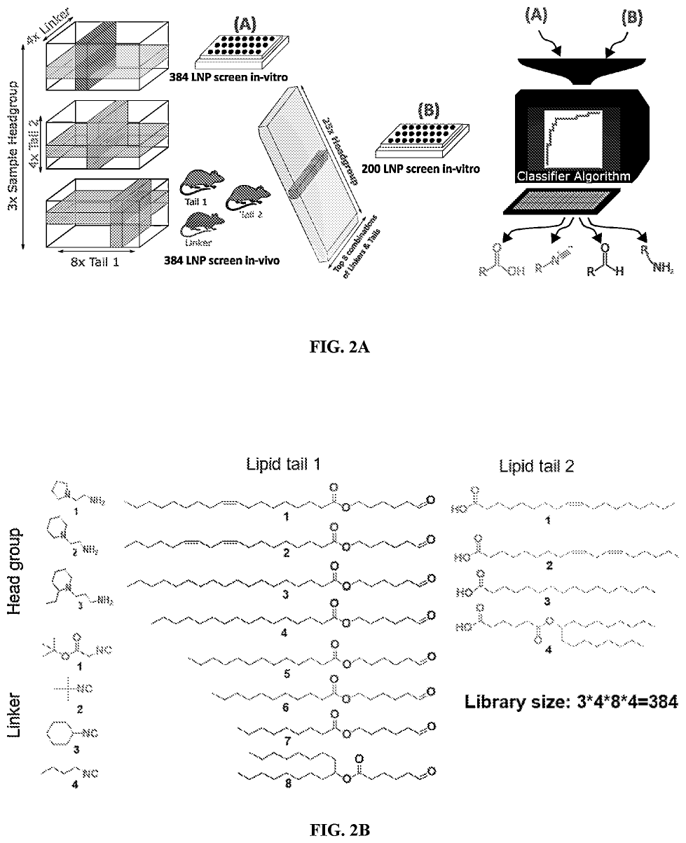

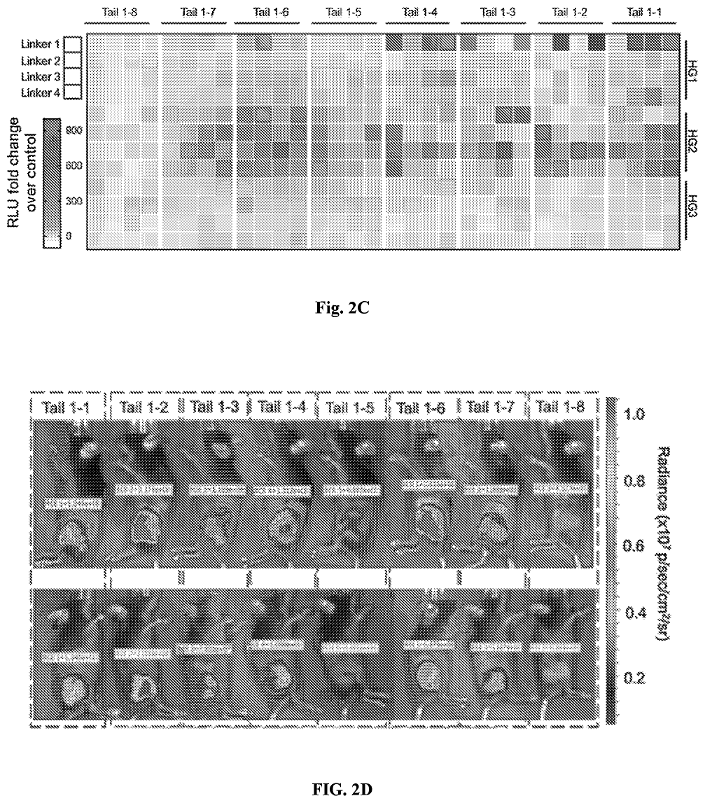

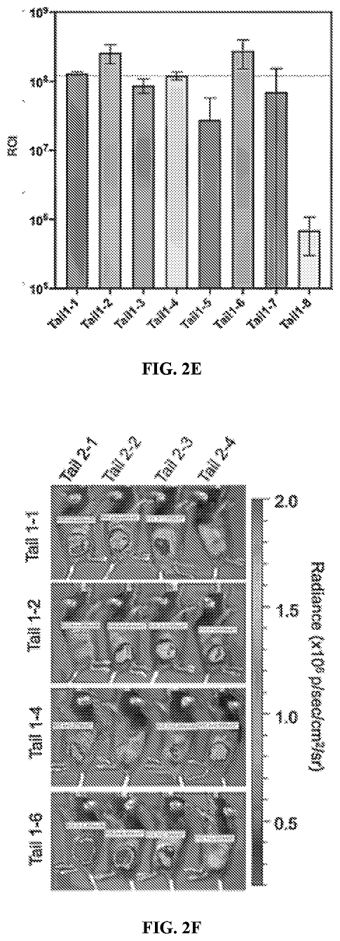

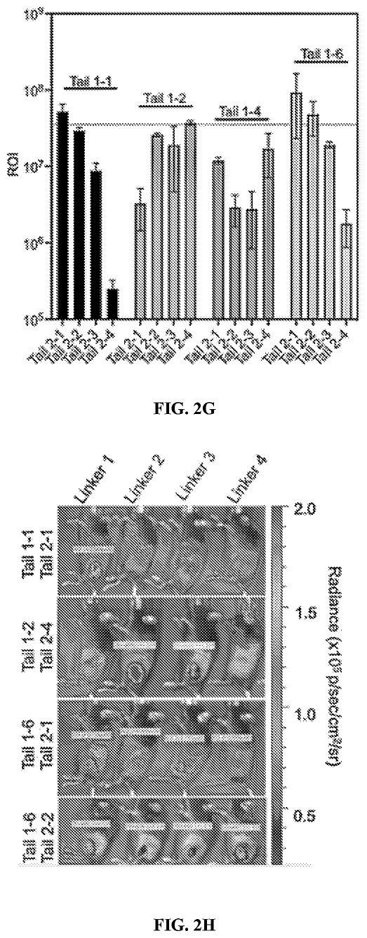

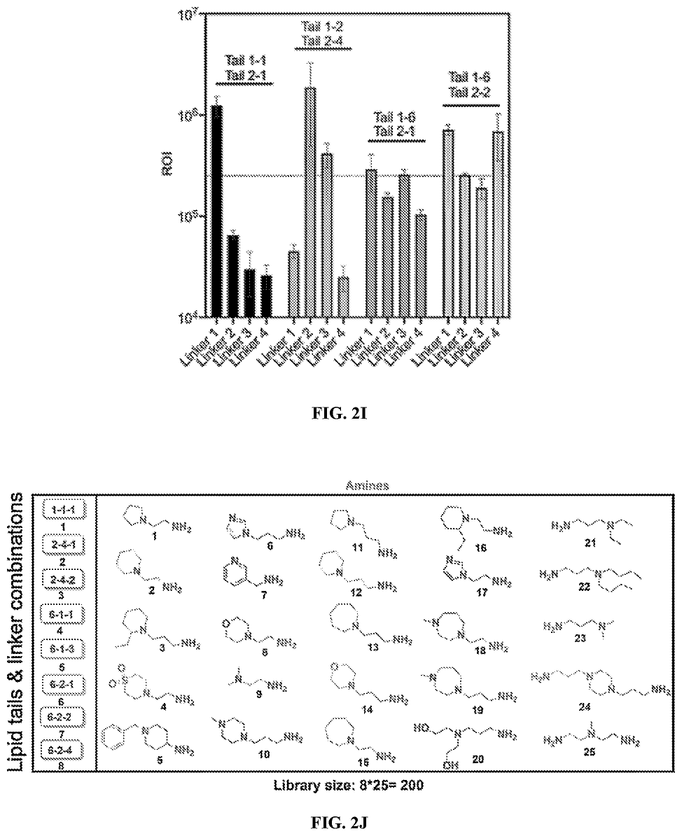

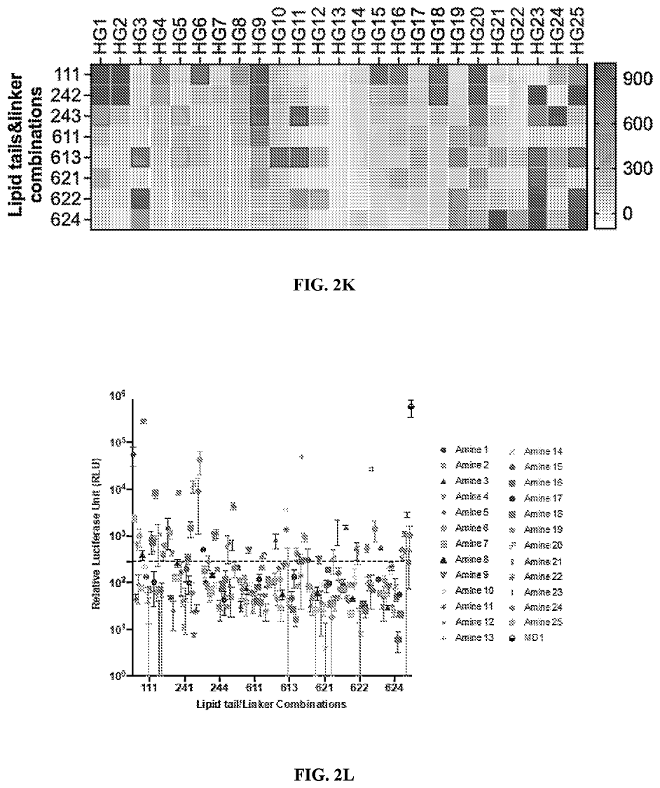

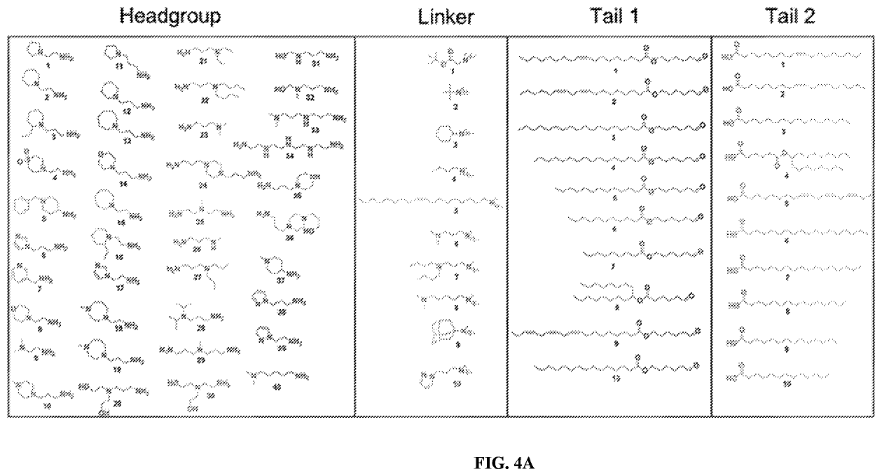

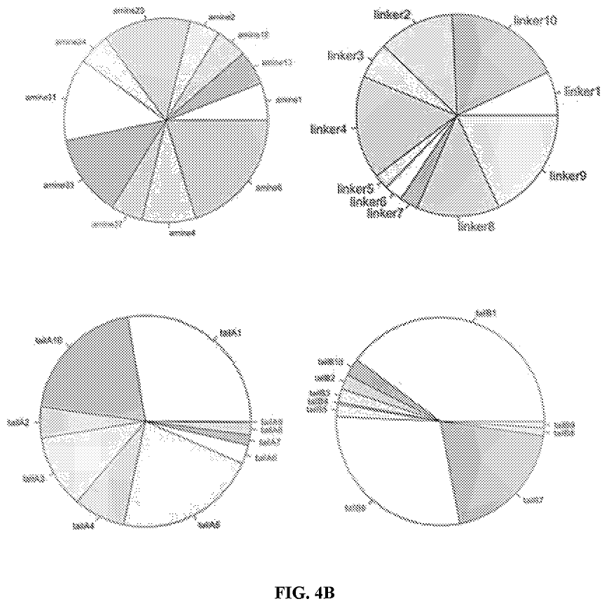

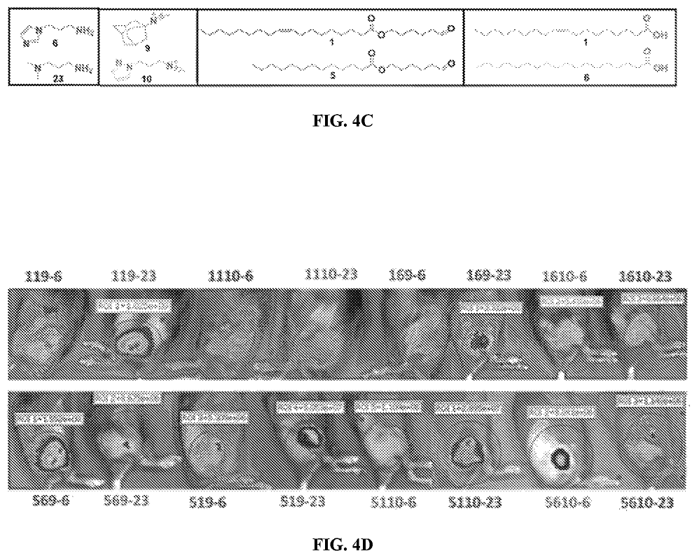

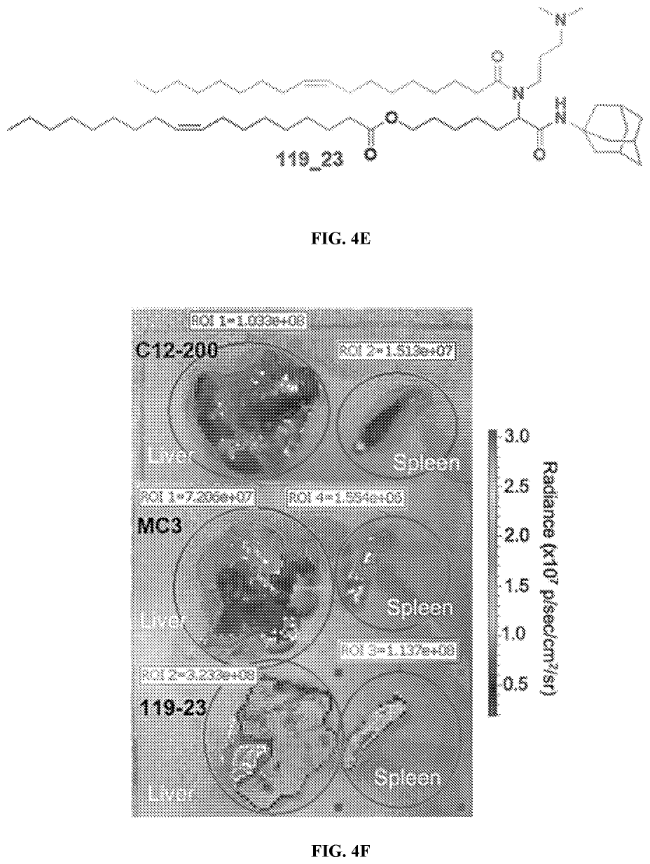

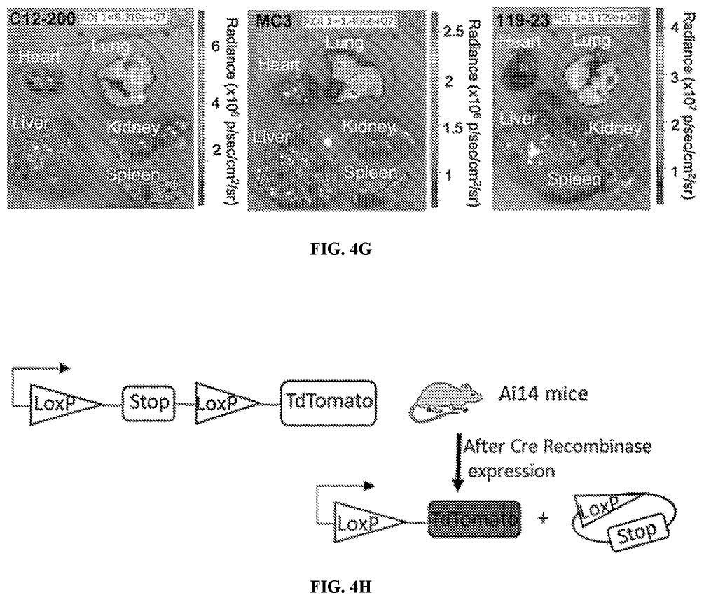

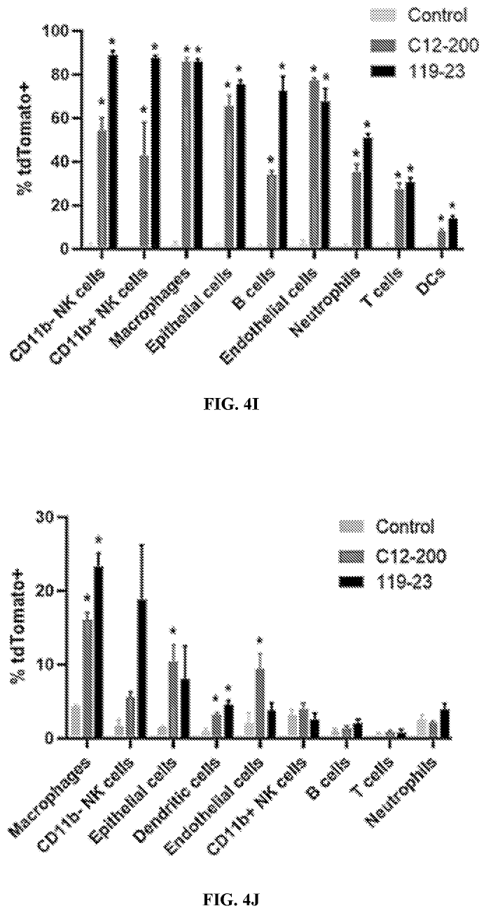

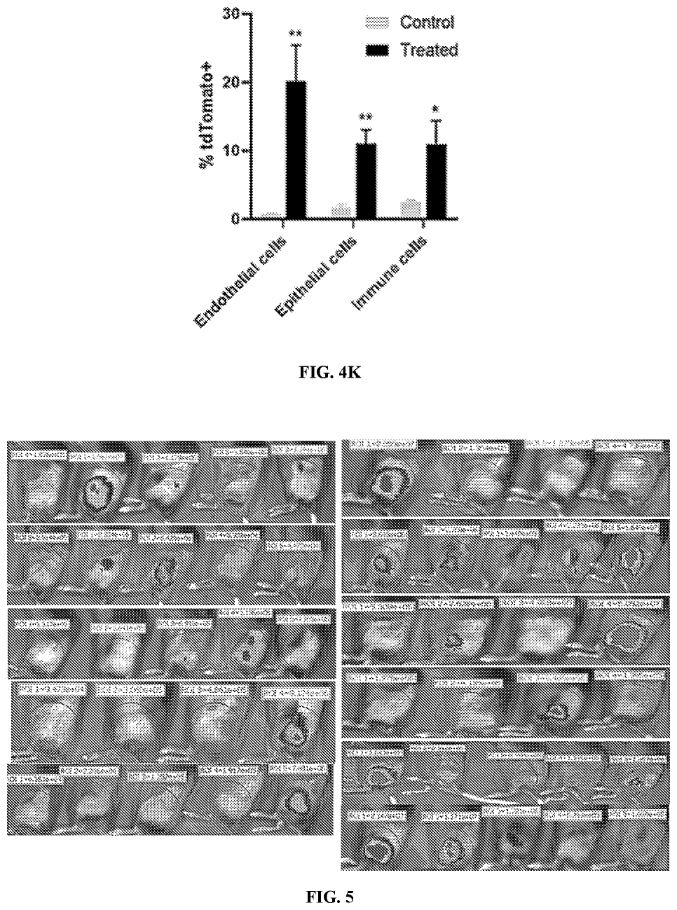

In certain embodiments, the compound of Formula (I) is an ionizable lipid that is a product of the four-component synthesis disclosed in FIG. 1B. In certain embodiments, the compound of Formula (I) is an ionizable lipid that is a product of the four-component synthesis disclosed in FIG. 1B using the components disclosed in any of FIGS. 2B, 2J, 4A, 4C, and/or 8A. In certain embodiments, the compound of Formula (I) is an ionizable lipid that is a product of the four-component synthesis disclosed in FIG. 1B, and is represented by a formula WXY—Z or WXY_Z, wherein W is the number of the aldehyde used for Tail 1, X is the number of the carboxylic acid used for Tail 2, Y is the number of the isoyanide used for the Linker, and Z is the number of the amine used for the Headgroup. In certain embodiments, the compound of Formula (I) is an ionizable lipid that is a product of the four-component synthesis disclosed in FIG. 1B using the components disclosed in any of FIGS. 2B, 2J, 4A, 4C, and/or 8A, and is represented by a formula WXY—Z or WXY_Z, wherein W is the number of the aldehyde component used for Tail 1, X is the number of the carboxylic acid component used for Tail 2, Y is the number of the isoyanide component used for the Linker, and Z is the number of the amine component used for the Headgroup. For example, compound “119-23” or “119_23” may refer to a compound of Formula (I) that is a product of the four-component synthesis disclosed in FIG. 1B represented by the formula WXY—Z or WXY_Z, wherein W is 1 (e.g., aldehyde 1 in FIG. 4A is used for Tail 1), X is 1 (e.g., carboxylic acid 1 in FIG. 4A is used for Tail 2), Y is 9 (e.g., isocyanide 9 in FIG. 4A is used for the Linker), and Z is 23 (e.g., amine 23 in FIG. 4A is used for the Headgroup). As a further example, compound “242-23” or “242_23” may refer to a compound of Formula (I) that is a product of the four-component synthesis disclosed in FIG. 1B represented by the formula WXY—Z or WXY_Z, wherein W is 2 (e.g., aldehyde 2 in FIG. 4A is used for Tail 1), X is 4 (e.g., carboxylic acid 4 in FIG. 4A is used for Tail 2), Y is 2 (e.g., isocyanide 9 in FIG. 4A is used for the Linker), and Z is 23 (e.g., amine 23 in FIG. 4A is used for the Headgroup).

### Compositions and Kits

The present disclosure provides compositions (e.g., pharmaceutical compositions) comprising a compound described herein, and an excipient (e.g., pharmaceutically acceptable excipient). In certain embodiments, the composition is a pharmaceutical composition. In certain embodiments, the composition is a cosmetic composition. In certain embodiments, the composition is a nutraceutical composition. In certain embodiments, the composition is a composition with a non-medical application. In certain embodiments, the excipient is a pharmaceutically acceptable excipient. In certain embodiments, the composition further comprises an active ingredient (e.g., RNA, small molecule compound).

Compositions described herein can be prepared by any method known in the art. In general, such preparatory methods include bringing the compound described herein into association with one or more excipients, and may include one or more agents and then, if necessary and/or desirable, shaping, and/or packaging the product into a desired single- or multi-dose unit. In certain embodiments, the agent and the compound of the composition are not covalently attached.

In certain embodiments, the composition is in the form of a particle. In certain embodiments, the particle is a nanoparticle or a microparticle. In certain embodiments, the particle is a micelle, liposome, or lipoplex. In certain embodiments, the particle encapsulates an agent, as described herein. In certain embodiments, the particle facilitates delivery of the agent into a cell. In certain embodiments, the particle facilitates delivery of the agent to a subject, e.g., a human.

### Lipid Nanoparticle Formulations

The present disclosure provides formulations (e.g., pharmaceutical compositions) comprising an ionizable compound (e.g., a compound of Formula (I) or (II)) described herein, and helper lipids (e.g., lipids that contribute to the stability and delivery efficiency of formulations (e.g., fatty acids or cholesteryl hemisuccinate (CHEMS)), phospholipids (e.g., non-cationic phospholipids), lipids for membrane structure (e.g., sterols), and PEG-lipids. In certain embodiments, the formulation is a pharmaceutical formulation. In certain embodiments, the formulation is a nanoparticle formulation comprising lipids (a “lipid nanoparticle formulation”). In certain embodiments, the formulation is in the form of a particle (e.g., nanoparticle).

In certain embodiments, the formulations comprise an ionizable compound described herein, and one or more of the following types of lipids: helper lipids (e.g., lipids that contribute to the stability and delivery efficiency of formulations (e.g., fatty acids or cholesteryl hemisuccinate (CHEMS))), phospholipids (e.g., non-cationic phospholipids), lipids for membrane structure (e.g., sterols), and PEG-lipids. In certain embodiments, the formulations comprise an ionizable compound described herein, and two or more of the following types of lipids: helper lipids (e.g., lipids that contribute to the stability and delivery efficiency of formulations (e.g., fatty acids or cholesteryl hemisuccinate (CHEMS))), phospholipids (e.g., non-cationic phospholipids), lipids for membrane structure (e.g., sterols), and PEG-lipids. In certain embodiments, the formulations comprise an ionizable compound described herein, and three or more of the following types of lipids: helper lipids (e.g., lipids that contribute to the stability and delivery efficiency of formulations (e.g., fatty acids or cholesteryl hemisuccinate (CHEMS))), phospholipids (e.g., non-cationic phospholipids), lipids for membrane structure (e.g., sterols), and PEG-lipids.

In certain embodiments, helper lipids are lipids that contribute to the stability and delivery efficiency of formulations. In certain embodiments, the helper lipid is a fatty acid. In certain embodiments, the helper lipid is oleic acid. In certain embodiments, the helper lipid is a neutral phospholipid. In certain embodiments, the helper lipid is a phosphatidylethanolamine. In certain embodiments, the helper lipid is dioleoylphosphatidylethanolamine (DOPE). In certain embodiments, the helper lipid is 1,2-Distearoylphosphatidylethanolamine (DSPE). In certain embodiments, the helper lipid is a phosphatidylcholine. In certain embodiments, the helper lipid is 1,2-Distearoyl-sn-glycero-3-phosphocholine (DSPC). In certain embodiments, the helper lipid is 1-palmitoyl-2-oleoyl-sn-glycero-3-phosphocholine (POPC or GPCho). In certain embodiments, the helper lipid is 1,2 Dipalmitoylphosphatidylcholine (DPPC). In certain embodiments, the helper lipid is DOPE or DSPC. In certain embodiments, the helper lipid is cholesteryl hemisuccinate (CHEMS).

In some embodiments, the helper lipid is a fixed cationic lipid or salt thereof. In some embodiments, the fixed cationic lipid is 1,2-dioleoyl-3-trimethylammonium propane (DOTAP), 1,2-di-O-octadecenyl-3-trimethylammonium propane (DOTMA), 1,2-stearoyl-3-trimethylammonium-propane (18:0 TAP), 1,2-dipalmitoyl-3-trimethylammonium-propane (16:0 TAP), 1,2-dimyristoyl-3-trimethylammonium-propane (14:0 TAP), dimethyldioctadecylammonium (18:0 DDAB), 1,2-dimyristoleoyl-sn-glycero-3-ethylphosphocholine (14:1 EPC), 1-palmitoyl-2-oleoyl-sn-glycero-3-ethylphosphocholine (16:0-18:1 EPC), 1,2-dioleoyl-sn-glycero-3-ethylphosphocholine (18:1 EPC), 1,2-distearoyl-sn-glycero-3-ethylphosphocholine (18:0 EPC), 1,2-dipalmitoyl-sn-glycero-3-ethylphosphocholine (16:0 EPC), 1,2-dimyristoyl-sn-glycero-3-ethylphosphocholine (14:0 EPC), 1,2-dilauroyl-sn-glycero-3-ethylphosphocholine (12:0 EPC), O,O′-ditetradecanoyl-N-(a-trimethylammonioacetyl)diethanolamine (DC-6-14), or N-(2-hydroxyethyl)-N,N-dimethyl-2,3-bis(oleoyloxy)propan-1-aminium. In some embodiments, the fixed cationic lipid is 1,2-dioleoyl-3-trimethylammonium propane (DOTAP).

In some embodiments, the helper lipid is a salt of a fixed cationic ligand. In certain embodiments, the salt of a fixed cationic lipid is a chloride salt, bromide salt, methyl sulfate salt, or triflate salt. In some embodiments, the salt of a fixed cationic ligand is a chloride salt.

In some embodiments, the helper lipid is an ionizable lipid. In certain embodiments, the ionizable lipid is 1,2-distearoyl-3-dimethylammonium-propane (18:0 DAP), 1,2-dipalmitoyl-3-dimethylammonium-propane (16:0 DAP), 1,2-dimyristoyl-3-dimethylammonium-propane (14:0 DAP), 1,2-dioleoyl-3-dimethylammonium-propane (DODAP or 18:1 DAP), or 1,2-dioleyloxy-3-dimethylaminopropane (DODMA).

In certain embodiments, the phospholipid is a non-cationic phospholipid. In certain embodiments, the non-cationic phospholipid is DSPC. In certain embodiments, the lipid for membrane structure is a sterol. In certain embodiments, the lipid for membrane structure is an animal sterol. In certain embodiments, the lipid for membrane structure is cholesterol. In certain embodiments, the PEG-lipid is a lipid that comprises polyethylene glycol (PEG). In certain embodiments, the PEG-lipid is an mPEG-comprising phospholipid. In certain embodiments, the PEG-lipid is a phospholipid comprising PEG2000 (PEG with a molecular weight of 2000 g/mol). In certain embodiments, the PEG-lipid is 1,2-dimyristoyl-sn-glycero-3-phosphoethanolamine-N-[methoxy-(polyethyleneglycol)-2000](ammonium salt) (“C14-PEG 2000”). In certain embodiments, the PEG-lipid is 1,2-dipalmitoyl-sn-glycero-3-phosphoethanolamine-N-[methoxy(polyethylene glycol)-2000](ammonium salt) (“16:0 PEG2000 phospholipid”). In certain embodiments, the PEG-lipid is 1,2-dioleoyl-sn-glycero-3-phosphoethanolamine-N-[methoxy(polyethylene glycol)-2000](ammonium salt) (“18:1 PEG2000 phospholipid”). In certain embodiments, the PEG-lipid is 1,2-distearoyl-sn-glycero-3-phosphoethanolamine-N-[methoxy(polyethylene glycol)-2000](ammonium salt) (“18:0 PEG2000 phospholipid”). In certain embodiments, the PEG-lipid is a phospholipid comprising PEG750 (PEG with a molecular weight of 750 g/mol), PEG1000 (PEG with a molecular weight of 1000 g/mol), PEG3000 (PEG with a molecular weight of 3000 g/mol), PEG3000 (PEG with a molecular weight of 3000 g/mol), or PEG5000 (PEG with a molecular weight of 5000 g/mol).

In certain embodiments, the formulations comprise a weight ratio of the lipid compounds described herein (“lipids”) to the mRNA of approximately 10:1. In certain embodiments, the formulations comprise a lipid to mRNA weight ratio of approximately 18:1, approximately 17:1, approximately 16:1, approximately 15:1, approximately 14:1, approximately 13:1, approximately 12:1, approximately 11:1, approximately 10:1, approximately 9:1, approximately 8:1, approximately 7:1, approximately 6:1, approximately 5:1, approximately 3:1, or approximately 2:1. In certain embodiments, the formulations comprise a lipid to mRNA weight ratio of approximately 15:1 to approximately 5:1. In certain embodiments, the formulations comprise a lipid to mRNA weight ratio of approximately 15:1 to approximately 10:1. In certain embodiments, the formulations comprise a lipid to mRNA weight ratio of approximately 12:1 to approximately 8:1, approximately 11:1 to approximately 8:1, approximately 11:1 to approximately 9:1, approximately 10:1 to approximately 8:1, or approximately 10:1 to approximately 9:1. In certain embodiments, the formulations comprise a lipid to mRNA weight ratio of approximately 10:1.

In certain embodiments, the formulations comprise approximately 3-30 molar % (“mol %”) of a helper lipid. In certain embodiments, the formulations comprise approximately 3-30 mol %, approximately 5-30 mol %, approximately 5-25 mol %, approximately 5-20 mol %, approximately 5-15 mol %, approximately 10-15 mol %, approximately 10-12 mol %, approximately 12-15 mol %, approximately 12-18 mol %, or approximately 12-20 mol % of a helper lipid. In certain embodiments, the formulations comprise approximately 3-30 mol % of a helper lipid. In certain embodiments, the formulations comprise approximately 10-20 mol % of DOPE. In certain embodiments, the formulations comprise 3-30 mol %, approximately 5-30 mol %, approximately 5-25 mol %, approximately 5-20 mol %, approximately 5-15 mol %, approximately 10-15 mol %, approximately 10-12 mol %, approximately 12-15 mol %, approximately 12-18 mol %, or approximately 12-20 mol % of DOPE.

In certain embodiments, the formulations comprise approximately 35-75 mol % of lipids described herein. In certain embodiments, the formulations comprise approximately 35-75 mol %, approximately 35-70 mol %, approximately 35-65 mol %, approximately 35-60 mol %, approximately 35-55 mol %, approximately 35-50 mol %, approximately 35-52 mol %, approximately 35-45 mol %, or approximately 35-40 mol % of lipids. In certain embodiments, the formulations comprise approximately 25-60 mol %, approximately 30-60 mol %, approximately 35-60 mol %, approximately 33-60 mol %, approximately 33-58 mol %, approximately 33-57 mol %, approximately 33-55 mol %, approximately 30-45 mol %, approximately 35-45 mol %, or approximately 35-50 mol % of lipids. In certain embodiments, the formulations comprise approximately 35-50 mol % of lipids. In certain embodiments, the formulations comprise approximately 30 mol %, approximately 31 mol %, approximately 32 mol %, approximately 33 mol %, approximately 34 mol %, approximately 35 mol %, approximately 36 mol %, approximately 37 mol %, approximately 38 mol %, approximately 39 mol %, approximately 40 mol %, approximately 41 mol %, approximately 42 mol %, approximately 43 mol %, approximately 44 mol %, approximately 45 mol %, approximately 46 mol %, approximately 47 mol %, approximately 48 mol %, approximately 49 mol %, or approximately 50 mol % of lipids.

In certain embodiments, the formulations comprise approximately 0.5-3.0 mol % of PEG-lipid. In certain embodiments, the formulations comprise approximately 0.5-3.0 mol %, approximately 0.5-2.5 mol %, 1.0-3.0 mol %, approximately 1.0-2.8 mol %, approximately 1.0-2.5 mol %, approximately 1.5-2.5 mol %, or approximately 1.5-2.0 mol % of PEG-lipid.

In certain embodiments, the formulations comprise approximately 1.0-3.0 mol %, approximately 1.2-2.8 mol %, 1.5-2.8 mol %, or approximately 1.5-2.5 mol % of PEG-lipid. In certain embodiments, the formulations comprise approximately 1.5 mol %, approximately 1.75 mol %, approximately 2.0 mol %, approximately 2.25 mol %, or approximately 2.5 mol % of PEG-lipid. In certain embodiments, the PEG-lipid is 1,2-dimyristoyl-sn-glycero-3-phosphoethanolamine-N-[methoxy-(polyethyleneglycol)-2000](ammonium salt) (“C14-PEG 2000”), and the formulations comprise approximately 1.5 mol %, approximately 1.75 mol %, approximately 2.0 mol %, approximately 2.25 mol %, or approximately 2.5 mol % of PEG-lipid. In certain embodiments, the PEG-lipid is 1,2-dimyristoyl-sn-glycero-3-phosphoethanolamine-N-[methoxy-(polyethyleneglycol)-2000](ammonium salt) (“C14-PEG 2000”), and the formulations comprise approximately 1.5-2.5 mol % PEG-lipid.

In certain embodiments, the formulations comprise a lipid to mRNA weight ratio of approximately 10:1; approximately 10-20 mol % of helper lipid; approximately 35-50 mol % of lipid; approximately 1.5-2.5 mol % of PEG-lipid. In certain embodiments, the formulations comprise a lipid to mRNA weight ratio of approximately 10:1; approximately 10-20 mol % of helper lipid DOPE; approximately 35-50 mol % of lipid; or approximately 1.5-2.5 mol % of PEG-lipid C14-PEG 2000.

In certain embodiments, the formulations comprise a lipid composition weight ratio of lipid/helper lipid/sterol/PEG-lipid of 45/15/45/2.0. In certain embodiments, the formulations comprise a lipid composition weight ratio of lipid/helper lipid/sterol/PEG-lipid of 45/10/42.5/2.5, wherein the helper lipid is DOPE, the sterol is cholesterol, and the PEG-lipid is C14-PEG 2000. In certain embodiments, the formulations comprise a lipid composition weight ratio of lipid/helper lipid/sterol/PEG-lipid of 45/15/45/2.0; 40/15/43/2.5; 35/12/43/2.5; 35/16/37/2.5; or 35/16/37/2.5.

In certain embodiments, the formulations comprise one or more agents (e.g., a polynucleotide) described herein. In certain embodiments, the agent is RNA. In certain embodiments, the agent is mRNA. In certain embodiments, the agent is an mRNA vaccine.

In certain embodiments, the composition comprises a compound of Formula (I), pharmaceutically acceptable salt, co-crystal, tautomer, stereoisomer, solvate, hydrate, polymorph, or isotopically enriched derivative thereof, a polunucleotide, and one or more of a helper lipid, a PEG-lipid, and a sterol. In certain embodiments, the composition comprises a compound of Formula (I), pharmaceutically acceptable salt, co-crystal, tautomer, stereoisomer, solvate, hydrate, polymorph, or isotopically enriched derivative thereof; a polunucleotide, and two or more of a helper lipid, a PEG-lipid, and a sterol. In certain embodiments, the composition comprises a compound of Formula (I), pharmaceutically acceptable salt, co-crystal, tautomer, stereoisomer, solvate, hydrate, polymorph, or isotopically enriched derivative thereof, a helper lipid, a PEG-lipid, a sterol, and a polynucleotide. In certain embodiments, the helper lipid is DOPE, the PEG-lipid is DMG-PEG2000, the sterol is cholesterol, and the polynucleotide is mRNA. In certain embodiments, the composition further comprises a cationic lipid. In certain embodiments, the cationic lipid is DOTAP. In certain embodiments, the compound is 119-23:

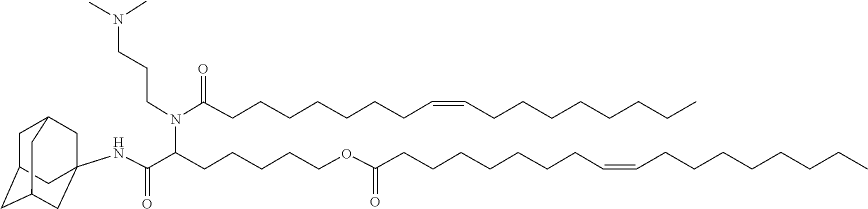 [SMILES: CCCCCCCC/C=C\CCCCCCCC(=O)OCCCCCC(C(=O)NC12CC3CC(CC(C3)C1)C2)N(CCCN(C)C)C(=O)CCCCCCC/C=C\CCCCCCCC]

or a pharmaceutically acceptable salt, co-crystal, tautomer, stereoisomer, solvate, hydrate, polymorph, or isotopically enriched derivative thereof. In certain embodiments, the compound is 242-23:

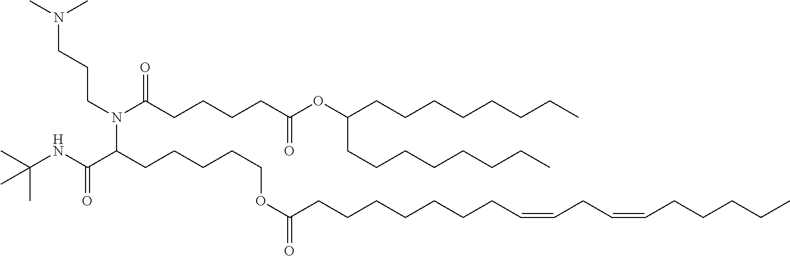 [SMILES: CCCCC/C=C\C/C=C\CCCCCCCC(=O)OCCCCCC(C(=O)NC(C)(C)C)N(CCCN(C)C)C(=O)CCCCC(=O)OC(CCCCCCCC)CCCCCCCC]

or a pharmaceutically acceptable salt, co-crystal, tautomer, stereoisomer, solvate, hydrate, polymorph, or isotopically enriched derivative thereof.

In certain embodiments, the formulations selectively deliver an agent to a tissue or organ in a subject (e.g., to lung, spleen, or liver tissue). In certain embodiments, the formulations selectively deliver an agent to lung tissue in a subject. In certain embodiments, the formulations selectively deliver an agent to spleen tissue in a subject. In certain embodiments, formulations selectively deliver an agent to liver tissue in a subject.

Formulations described herein can be prepared by any method known in the art.

Compositions can be prepared, packaged, and/or sold in bulk, as a single unit dose, and/or as a plurality of single unit doses. A “unit dose” is a discrete amount of the composition comprising a predetermined amount of the agent. The amount of the agent is generally equal to the dosage of the agent which would be administered to a subject and/or a convenient fraction of such a dosage, such as one-half or one-third of such a dosage.

Relative amounts of the compound, excipient, agent, and/or any additional ingredients in a composition described herein will vary, depending upon the identity, size, and/or condition of the subject treated and further depending upon the route by which the composition is to be administered. The composition may comprise between 0.1% and 100% (w/w) agent. The composition may comprise no agent.

Excipients and accessory ingredients used in the manufacture of provided compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter, PEGylated lipids, phospholipids, suppository waxes, coloring agents, coating agents, sweetening, flavoring, and perfuming agents, may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol©), sucrose fatty acid esters, polyethylene glycol fatty acid esters (e.g., Cremophor©), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

In certain embodiments, the compositions further comprise an agent and are useful for delivering said agent (e.g., to a subject or cell). In certain embodiments, the compositions are pharmaceutical compositions which are useful for treating a disease in a subject in need thereof. In certain embodiments, the pharmaceutical compositions are useful for preventing a disease in a subject.

A composition as described herein may further comprise, or can be administered in combination with, one or more additional agents. In certain embodiments, the agent is a small organic molecule, inorganic molecule, nucleic acid, protein, peptide, or polynucleotide. In certain embodiments, the agent is a pharmaceutical agent (e.g., therapeutically and/or prophylactically active agent). Pharmaceutical agents include therapeutically active agents.

Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, polynucleotides, lipids, hormones, vitamins, vaccines, immunological agents, and cells or other biological materials.

In certain embodiments, the agent is a polynucleotide. In certain embodiments, the polynucleotide is DNA. In certain embodiments, the polynucleotide is RNA. In certain embodiments, the polynucleotide carries out RNA interference. The RNA is selected from the group consisting of double-stranded RNA (dsRNA), small interfering RNA (siRNA), short hairpin (shRNA), microRNA (miRNA), messenger RNA (mRNA), antisense RNA, transfer RNA (tRNA), small nuclear RNA (snRNA), and ribosomal RNA (rRNA). In certain embodiments, the RNA is dsRNA. In certain embodiments, the RNA is siRNA. In certain embodiments, the RNA is shRNA. In certain embodiments, the RNA is miRNA. In certain embodiments, the RNA is mRNA. In certain embodiments, the mRNA encodes proteins (e.g., peptide and protein structures, allowing expression of the entire antigen). In certain embodiments, the mRNA encodes an antigen. In certain embodiments, the mRNA encodes a Class I Major Histocompatibility Complex (MHC) antigen. In certain embodiments, the mRNA encodes a Class II MHC antigen. In certain embodiments, the mRNA encodes a Class I and II MHC antigen. In certain embodiments, the RNA is an mRNA vaccine. In certain embodiments, the RNA is antisense RNA. In certain embodiments, the RNA is dsRNA, siRNA, shRNA, miRNA, mRNA, or antisense RNA.

In certain embodiments, the agent described herein is provided in an effective amount in the composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating an autoimmune disease in a subject in need thereof. In certain embodiments, the effective amount is an amount effective for preventing an autoimmune disease in a subject in need thereof. In certain embodiments, the effective amount is an amount effective for reducing the risk of developing a disease (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject in need thereof.

Compositions may be formulated into liquid dosage forms for oral and parenteral administration include pharmaceutically acceptable emulsions, microemulsions, solutions, suspensions, syrups, and elixirs. In addition to the agents, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the particles described herein are mixed with solubilizing agents, such as Cremophor*, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids, such as oleic acid, are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Dosage forms for topical and/or transdermal administration of a composition described herein may include ointments, pastes, creams, lotions, gels, powders, solutions, sprays, inhalants, and/or patches. Generally, the agent is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an agent to the body. Such dosage forms can be prepared, for example, by dissolving and/or dispensing the agent in the proper medium. Alternatively or additionally, the rate can be controlled by either providing a rate controlling membrane and/or by dispersing the agent in a polymer matrix and/or gel.

Suitable devices for use in delivering intradermal compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the polymer in powder form through the outer layers of the skin to the dermis are suitable.

Formulations suitable for topical administration include liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) agent, although the concentration of the agent can be as high as the solubility limit of the agent in the solvent. Formulations for topical administration may further comprise one or more of the additional ingredients described herein.

A composition described herein can be prepared, packaged, and/or sold in a formulation suitable for pulmonary administration via the buccal cavity. Such a formulation may comprise dry particles which comprise the agent and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the agent dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the agent may constitute 0.1 to 20% (w/w) of the composition. The propellant may further comprise additional ingredients such as a liquid non-ionic and/or solid anionic surfactant and/or a solid diluent (which may have a particle size of the same order as particles comprising the agent).

Compositions described herein formulated for pulmonary delivery may provide the agent in the form of droplets of a solution and/or suspension. Such formulations can be prepared, packaged, and/or sold as aqueous and/or dilute alcoholic solutions and/or suspensions, optionally sterile, comprising the agent, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate.

Formulations described herein as being useful for pulmonary delivery are useful for intranasal delivery of a pharmaceutical composition described herein. Another formulation suitable for intranasal administration is a coarse powder comprising the agent and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the agent, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) agent, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the agent. Such powdered, aerosolized, and/or aerosolized formulations, when dispersed, may have an average particle and/or droplet size in the range from about 0.1 to about 200 nanometers, and may further comprise one or more of the additional ingredients described herein.

A composition described herein can be prepared, packaged, and/or sold in a formulation for ophthalmic administration. Such formulations may, for example, be in the form of eye drops including, for example, a 0.1-1.0% (w/w) solution and/or suspension of the agent in an aqueous or oily liquid carrier or excipient. Such drops may further comprise buffering agents, salts, and/or one or more other of the additional ingredients described herein. Other opthalmically-administrable formulations which are useful include those which comprise the agent in microcrystalline form and/or in a liposomal preparation. Ear drops and/or eye drops are also contemplated as being within the scope of this disclosure.

Although the descriptions of compositions provided herein are principally directed to compositions which are suitable for administration to humans, it will be understood by the skilled artisan that such compositions are generally suitable for administration to animals of all sorts. Modification of compositions suitable for administration to humans in order to render the compositions suitable for administration to various animals is well understood, and the ordinarily skilled veterinary pharmacologist can design and/or perform such modification with ordinary experimentation.

Compositions provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific agent employed; the specific composition employed; the age, body weight, general health, sex, and diet of the subject; the time of administration, route of administration, and rate of excretion of the specific agent employed; the duration of the treatment; drugs used in combination or coincidental with the specific agent employed; and like factors well known in the medical arts.

The compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 pg and 1 pg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

The lipids in the compositions can be administered in combination with additional agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a composition described herein including a lipid compound described herein and an agent shows a synergistic effect that is absent in a composition including one of the lipid compound and an agent, but not both.

The composition can be administered concurrently with, prior to, or subsequent to one or more additional agents (e.g., a pharmaceutical agent), in addition to the lipid in the composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease). Each additional pharmaceutical agent may be administered at a dose and/or on a time schedule determined for that pharmaceutical agent. The additional pharmaceutical agents may also be administered together with each other and/or with the polymer or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or compositions (e.g., pharmaceutical compositions) can be administered in combination with an anti-cancer therapy including surgery, radiation therapy, transplantation (e.g., stem cell transplantation, bone marrow transplantation), immunotherapy, and chemotherapy.

In some embodiments, the composition is a particle (e.g., a nanoparticle). In some embodiments, the particle is substantially soluble in water (e.g., hydrophilic). In some embodiments, the particle is substantially insoluble in water (e.g., hydrophobic). In some embodiments, the particle is substantially insoluble in water and greater than about 10,000 parts water are required to dissolve 1 part compound. In one embodiment, the particle is amphiphilic. In one embodiment, the particle comprises a segment that is hydrophobic and a segment that is hydrophilic.

In some embodiments, the percentage of the particle that comprises an agent is between about 1 and about 100% (e.g., about 1%, about 2%, about 3%, about 4%, about 5%, about 10%, about 15%, about 20%, about 30%, about 40%, about 50%, about 60%, about 70%, about 80%, about 90%, or about 100%). In some embodiments, the percentage of the particle that comprises an agent is less than about 50%, e.g., less than about 40%, less than about 35%, less than about 30%, less than about 25%, less than about 20%, less than about 15%, or less than about 10%. In some embodiments, the percentage of the particles that comprise an agent is between about 5% and about 50%, about 5% and about 40%, about 5% and about 30%, about 5% and about 25%, or about 5% and about 20%. In some embodiments, the percentage of the particle that comprises an agent is between about 5% and 90%. In some embodiments, the percentage of the particles that comprise an agent is between about 5% and about 75%. In the some embodiments, the percentage of particle that comprises an agent is between about 5% and about 50%. In the some embodiments, the percentage of the particle that comprises an agent is between about 10% and about 25%.

In some embodiments, the total amount of the agent present in the particle is greater than about 5% (e.g., about 6%, about 7%, about 8%, about 9%, about 10%, about 12%, about 15%, about 20%, about 25%, about 30%, or more) of the total size or weight of the conjugate or particle. In some embodiments, the total amount of the agent present in the conjugate or particle is greater than about 10% (e.g., about 12%, about 15%, about 20%, about 25%, about 30%, or more) of the total size or weight of the conjugate or particle.

Without being bound by theory, due to the localized delivery of the compositions described herein (e.g., the agent-containing particles), a lower dose or amount of the agent in the particles can be administered (e.g., through local sustained delivery) compared to the agent in free form. In other embodiments, the agent-containing particles are administered at a dose or amount of the agent that is less than the dose or amount of said agent in free form to have a desired effect (e.g., a desired therapeutic effect).

In some embodiments, the agent is incorporated into a particle at a dose that is less than the dose or amount of said agent in free form to have a desired effect (e.g., a desired therapeutic effect), e.g., the standard of care dose for the intended use of the free agent.

In another aspect, provided are kits including a first container comprising a compound or composition described herein and instructions for use. The kits may further comprise a container (e.g., a vial, ampule, bottle, syringe, and/or dispenser package, or other suitable container). In some embodiments, provided kits may optionally further include a second container comprising an excipient for dilution or suspension of a composition or polymer described herein. In some embodiments, the compositions described herein provided in the first container and the second container are combined to form one unit dosage form.

In certain embodiments, the kits are useful for delivering an agent (e.g., to a subject or cell). In certain embodiments, the kits are useful for selectively delivering an agent to a tissue or organ in a subject (e.g., to lung, spleen, or liver tissue). In certain embodiments, the kits are useful for selectively delivering an agent to lung tissue in a subject. In certain embodiments, the kits are useful for selectively delivering an agent to spleen tissue in a subject. In certain embodiments, the kits are useful for selectively delivering an agent to liver tissue in a subject. In certain embodiments, the kits are useful for treating a disease (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject in need thereof. In certain embodiments, the kits are useful for preventing a disease (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject in need thereof. In certain embodiments, the kits are useful for reducing the risk of developing a disease (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for delivering an agent. In certain embodiments, the kits and instructions provide for delivering an agent (e.g., RNA) to a cell or tissue in a subject (e.g., a lung, liver, or spleen cell/tissue). In certain embodiments, the kits and instructions provide for treating a disease (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject in need thereof. In certain embodiments, the kits and instructions provide for preventing a disease (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject in need thereof. In certain embodiments, the kits and instructions provide for reducing the risk of developing a disease (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease) in a subject in need thereof. A kit described herein may include one or more additional pharmaceutical agents described herein as a separate composition.

### Methods of Synthesis

The present disclosure provides methods for making a compound of Formula (I):

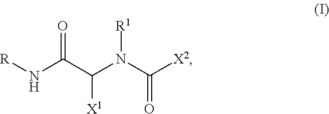

or a pharmaceutically acceptable salt, co-crystal, tautomer, stereoisomer, solvate, hydrate, polymorph, or isotopically enriched derivative thereof, the method comprising reacting:
an isocyanide of Formula (A):

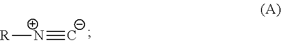

an aldehyde of Formula (B):

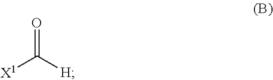

an amine of Formula (C):

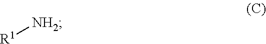

and
an acid of Formula (D):

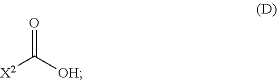

under suitable conditions to form a compound of Formula (I); wherein:
R is substituted or unsubstituted alkyl, substituted or unsubstituted heteroalkyl, or substituted or unsubstituted carbocyclyl;
R¹ is substituted or unsubstituted alkyl, substituted or unsubstituted heteroalkyl, or substituted or unsubstituted heterocyclyl; and
X¹ and X² are each independently substituted or unsubstituted C₈-C₁₀₀ aliphatic, or substituted or unsubstituted C₈-C₁₀₀ heteroaliphatic.

The present disclosure provides a compound of Formula (I):

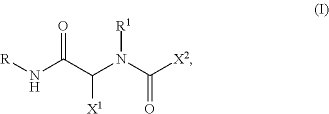

or a pharmaceutically acceptable salt thereof, synthesized by a method comprising reacting:
an isocyanide of Formula (A):

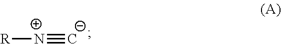

an aldehyde of Formula (B):

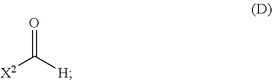

an amine of Formula (C):

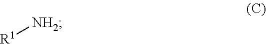

and
an acid of Formula (D):

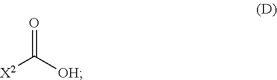

under suitable conditions to form a compound of Formula (I); wherein:
R is substituted or unsubstituted alkyl, substituted or unsubstituted heteroalkyl, or substituted or unsubstituted carbocyclyl;
R¹ is substituted or unsubstituted alkyl, substituted or unsubstituted heteroalkyl, or substituted or unsubstituted heterocyclyl; and
X¹ and X² are each independently substituted or unsubstituted C₈-C₁₀₀ aliphatic, or substituted or unsubstituted C₈-C₁₀₀ heteroaliphatic.

### Methods of Treatment and Uses

The present disclosure also provides methods of using the compounds described herein, or compositions (e.g., pharmaceutical compositions) or formulations thereof, for delivering an agent (e.g., a polynucleotide (e.g., RNA)). The present disclosure also provides methods of using the compounds described herein, or compositions (e.g., pharmaceutical compositions) thereof, for the treatment, prevention, or diagnosis of a disease or condition (e.g., genetic disease, proliferative disease, hematological disease, neurological disease, painful condition, psychiatric disorder, metabolic disorder, long-term medical condition, inflammatory disease, autoinflammatory disease, liver disease, lung disease, spleen disease, familial amyloid neuropathy, cardiovascular disease, viral infection, infectious disease, fibrotic condition, or autoimmune disease).

The present disclosure also provides a compound of Formula (I), (II), or a pharmaceutically acceptable salt, co-crystal, tautomer, stereoisomer, solvate, hydrate, polymorph, isotopically enriched derivative, or prodrug, or composition thereof, for use in the treatment of diseases, such as genetic diseases, proliferative diseases, hematological diseases, neurological diseases, painful condition, psychiatric disorder, metabolic disorder, long-term medical condition, inflammatory diseases, autoinflammatory diseases, liver diseases, lung diseases, spleen diseases, familial amyloid neuropathy, cardiovascular diseases, viral infection, infectious diseases, fibrotic condition, or autoimmune diseases, in a subject in need thereof. The present disclosure also provides a compound of Formula (I), (II), or a pharmaceutically acceptable salt, co-crystal, tautomer, stereoisomer, solvate, hydrate, polymorph, isotopically enriched derivative, or prodrug, or composition thereof, for use in the delivery of an agent (e.g., RNA) to a tissue or organ, in a subject in need thereof. In certain embodiments, provided herein are compositions including compounds described herein for delivering an agent. In certain embodiments, provided herein are compositions including compounds described herein for delivering an RNA agent.

In certain embodiments, the methods described herein include treating a disease, disorder, or condition from which a subject suffers, comprising administering to a subject in need thereof an effective amount of a composition described herein. In certain embodiments, the methods described herein include delivering an RNA agent to a tissue or cell (e.g., a liver, lung, or spleen tissue/cell), comprising administering to a subject in need thereof an effective amount of a composition described herein. In certain embodiments, the methods described herein selectively deliver an RNA agent to a tissue or organ in a subject (e.g., to lung, spleen, or liver tissue). In certain embodiments, the methods described herein selectively deliver an agent to lung tissue in a subject. In certain embodiments, the methods described herein selectively deliver an agent to spleen tissue in a subject. In certain embodiments, methods described herein selectively deliver an agent to liver tissue in a subject. In certain embodiments, the methods described herein include implanting in a subject an effective amount of the composition described herein. In certain embodiments, the methods described herein comprise treating a disease or condition in a subject in need thereof by administering to or implanting in the subject a therapeutically effective amount of a composition. In certain embodiments, the methods described herein comprise preventing a disease or condition in a subject in need thereof by administering to or implanting in the subject a prophylactically effective amount of a composition. In certain embodiments, the methods described herein comprise diagnosing a disease or condition in a subject in need thereof by administering to or implanting in the subject a diagnostically effective amount of a composition. In certain embodiments, the disease or condition is a genetic disease, proliferative disease, hematological disease, neurological disease, painful condition, psychiatric disorder, metabolic disorder, long-term medical condition, cancer (e.g. lung cancer, large bowel cancer, pancreas cancer, biliary tract cancer, or endometrial cancer), neoplasm, angiogenesis, inflammatory disease, autoinflammatory disease, liver disease, lung disease, spleen disease, familial amyloid neuropathy, cardiovascular disease, viral infection, infectious disease, fibrotic condition, or autoimmune disease. In some embodiments, the compositions are useful in treating cancer.

In some embodiments, the compositions are useful in treating lung cancer, head-and-neck cancer, esophagus cancer, stomach cancer, breast cancer, pancreas cancer, liver cancer, kidney cancer, prostate cancer, glioblastomas, metastatic melanomas, peritoneal or pleural mesotheliomas. In some embodiments, the compositions are useful in treating lung cancer. In some embodiments, the compositions are useful in treating non-small cell lung cancer. In some embodiments, the compositions are useful in treating breast cancer. In some embodiments, the compositions are useful in treating pancreas cancer. In some embodiments, the compositions are useful in treating liver cancer. In some embodiments, the compositions are useful in treating melanoma. In some embodiments, the compositions are useful in treating prostate cancer.

In some embodiments, the proliferative disease is a benign neoplasm. All types of benign neoplasms disclosed herein or known in the art are contemplated as being within the scope of the disclosure. In some embodiments, the proliferative disease is associated with angiogenesis. All types of angiogenesis disclosed herein or known in the art are contemplated as being within the scope of the disclosure. In certain embodiments, the proliferative disease is an inflammatory disease. All types of inflammatory diseases disclosed herein or known in the art are contemplated as being within the scope of the disclosure. In certain embodiments, the inflammatory disease is rheumatoid arthritis. In some embodiments, the proliferative disease is an autoinflammatory disease. All types of autoinflammatory diseases disclosed herein or known in the art are contemplated as being within the scope of the disclosure. In some embodiments, the proliferative disease is an autoimmune disease. All types of autoimmune diseases disclosed herein or known in the art are contemplated as being within the scope of the disclosure.

In certain embodiments, the disease is a cardiovascular disease. In certain embodiments, the disease is atherogenesis or atherosclerosis. In certain embodiments, the disease is arterial stent occlusion, heart failure (e.g., congestive heart failure), a coronary arterial disease, myocarditis, pericarditis, a cardiac valvular disease, stenosis, restenosis, in-stent-stenosis, angina pectoris, myocardial infarction, acute coronary syndromes, coronary artery bypass grafting, a cardio-pulmonary bypass procedure, endotoxemia, ischemia-reperfusion injury, cerebrovascular ischemia (stroke), renal reperfusion injury, embolism (e.g., pulmonary, renal, hepatic, gastro-intestinal, or peripheral limb embolism), or myocardial ischemia.

In certain embodiments, the disease is an infectious disease. In certain embodiments, the disease is a viral infection. In certain embodiments, the disease is an infection caused by DNA virus. In certain embodiments, the disease is an infection caused by a dsDNA virus. In certain embodiments, the disease is an infection caused by an ssDNA virus. In certain embodiments, the disease is an infection caused by an RNA virus. In certain embodiments, the disease is an infection caused by a dsRNA virus. In certain embodiments, the disease is an infection caused by a (+)ssRNA virus. In certain embodiments, the disease is an infection caused by a (−)ssRNA virus. In certain embodiments, the disease is an infection caused by a reverse transcribing (RT) virus. In certain embodiments, the disease is an infection caused by an ssRNA-RT virus. In certain embodiments, the disease is an infection caused by a dsDNA-RT virus. In certain embodiments, the disease is an infection caused by human immunodeficiency virus (HIV). In certain embodiments, the disease is an infection caused by acquired immunodeficiency syndrome (AIDS). In certain embodiments, the disease is an infection caused by human papillomavirus (HPV). In certain embodiments, the disease is an infection caused by hepatitis C virus (HCV). In certain embodiments, the disease is an infection caused by a herpes virus (e.g., herpes simplex virus (HSV)). In certain embodiments, the disease is an infection caused by Ebola virus. In certain embodiments, the disease is an infection caused by severe acute respiratory syndrome (SARS). In certain embodiments, the disease is an infection caused by influenza virus. In certain embodiments, the disease is an infection caused by an influenza virus. In certain embodiments, the disease is an infection caused by an influenza A virus. In certain embodiments, the disease is human flu (e.g., human flu caused by H1N1, H2N2, H3N2, H5N1, H7N7, H1N2, H9N2, H7N2, H7N3, or H10N7 virus). In certain embodiments, the disease is bird flu (e.g., bird flu caused by H5N1 or H7N9 virus). In certain embodiments, the disease is swine influenza (e.g., swine influenza caused by H1N1, H1N2, H2N1, H3N1, H3N2, H2N3, or influenza C virus). In certain embodiments, the disease is Dengue fever, Dengue hemorrhagic fever (DHF), Dengue shock syndrome (DSS), hepatitis A, hepatitis B, hepatitis D, hepatitis E, hepatitis F, infection caused by Coxsackie A virus, infection caused by Coxsackie B virus, fulminant viral hepatitis, viral myocarditis, infection caused by parainfluenza virus, infection caused by an RS virus (RSV) (e.g., RSV bronchiolitis, RSV pneumonia, especially an infant and childhood infection caused by RSV and RSV pneumonia in the patients with cardiopulmonary disorders), infection caused by measles virus, infection caused by vesicular stomatitis virus, infection caused by human metapneumovirus (HMPV), infection caused by rabies virus, Japanese encephalitis, infection caused by Junin virus, infection caused by human cytomegalovirus, infection caused by varicellovirus, infection caused by cytomegalovirus, infection caused by muromegalovirus, infection caused by proboscivirus, infection caused by roseolovirus, infection caused by lymphocryptovirus, infection caused by macavirus, infection caused by percavirus, infection caused by rhadinovirus, infection caused by poliovirus, infection caused by Zika virus, infection caused by Marburg virus, infection caused by Lassa fever virus, Venezuelan equine encephalitis, infection caused by Rift Valley Fever virus, infection caused by Korean hemorrhagic fever virus, infection caused by Crimean-Congo hemorrhagic fever virus, encephalitis, Saint Louise encephalitis, Kyasanur Forest disease, Murray Valley encephalitis, tick-borne encephalitis, West Nile encephalitis, yellow fever, infection caused by adenovirus, infection caused by poxvirus, or a viral infection in subjects with immune disorders.

In certain embodiments, the methods described herein include contacting a cell with an effective amount of a composition thereof. In certain embodiments, the cell is in vitro. In certain embodiments, the cell is in vivo. In certain embodiments, the cell is a liver cell. In certain embodiments, the cell is a spleen cell. In certain embodiments, the cell is a lung cell.

### Machine Learning Algorithms and Computer Systems

The present disclosure also provides a method for identifying an ionizable lipid useful for transfection when formulated into a composition. In some embodiments, the method comprises providing an input dataset, executing a machine learning algorithm that provides an output dataset, synthesizing an ionizable lipid provided in the output dataset, formulating the ionizable lipid into a composition, and determining a transfection potency of the composition, thereby identifying the ionizable lipid as useful for transfection when formulated into a composition. In some embodiments, the method further comprises producing a training dataset. In some embodiments, producing the training dataset comprises transforming an experimental dataset into binary outcomes. In some embodiments, providing an input dataset comprises calculating molecular features for each component. In some embodiments, providing an input dataset comprises applying one or more selection steps. In some embodiments, the one or more selection steps comprise removing features with low variance, removing empty/null values, and/or ensuring feature commonality between the old and new libraries. In some embodiments, the machine learning algorithm is random forest, logistic regression, or gradient boosting (e.g., XGBoost). In some embodiments, the machine learning algorithm is gradient boosting (e.g., XGBoost). In some embodiments, the composition comprises an agent. In some embodiments, the composition comprises a helper lipid, a PEG-lipid, a sterol, and a polynucleotide. In some embodiments, the transfection potency of the composition is measured using luminescence. In some embodiments, the transfection potency of the composition is greater than or equal to a transfection potency of a reference composition. In some embodiments, the reference composition comprises MC3.

The present disclosure also provides machine learning algorithms for identifying ionizable lipids. In certain embodiments, the ionizable lipids are of Formula (I). In some embodiments, the machine learning algorithm is for identifying ionizable lipids for use in compositions comprising an agent. In some embodiments, the machine learning algorithm is for identifying ionizable lipids for use in compositions comprising a helper lipid, a PEG-lipid, a sterol, and a polynucleotide. In some embodiments, the machine learning algorithm is for identifying ionizable lipids for use in compositions with increased transfection efficacy. In some embodiments, the machine learning algorithm is for identifying ionizable lipids for use in lipid nanoparticles.

The present disclosure also provides computer systems for identifying ionizable lipids, comprising one or more computer processors and storage having computer-useable instructions embodied thereon, wherein the computer-useable instructions are configured to perform a machine learning algorithm for identifying ionizable lipids. The present disclosure also provides one or more computer-readable hardware storage having computer-useable instructions embodied thereon and configured for performing a machine learning algorithm for identifying ionizable lipids.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Example 1. LNP Design

The Ugi four component reaction (4-CR) (FIG. 1) was used to develop a rapid and efficient four-component synthesis approach for ionizable lipids with distinct tails, comprising isocyanides (as linkers), aldehydes (as tail A), primary amines (as head groups) and carboxylic acids (as tail B) (FIG. 2). The UGI 4-CR method has multiple advantages: the higher dimensionality of the 4CR reaction allows it to produce a more diverse library of ionizable lipids more rapidly and with higher yield, expanding the possible lipid structures available for evaluation; the 4CR reaction system explores a much broader array of lipid tail combinations, which could further increase the structural diversity of final ionizable lipids; through careful library design, the ionizable lipids can be designed to be biodegradable, eliminating concerns with retention of lipids and related therapeutic toxicity; and the 4CR requires no additional reagents and is performed in biocompatible solvents without the need for purification.

While the Ugi 4CR reaction system can rapidly and efficiently generate thousands of lipids, screening their capability for mRNA delivery is time-consuming. A few hundred lipids were synthesized and screened to develop a sufficient dataset to train a machine learning algorithm and perform in silico screening of a more extensive library.

As shown in FIG. 2B, a library of 384 ionizable lipids (3 amine headgroups×4 isocyanide linkers x 8 aldehyde tail 1×4 carboxylic acid tail 2) was synthesized. Structural features such as degree of unsaturation and branching that facilitate the endosomal escape of RNA were adopted in the components of both tails, which were also designed to contain biodegradable ester groups⁸. The linker components were chosen to include both cyclic and acyclic isocyanides. Lipid synthesis in the high-throughput screening stage was conducted in 96-well plates.

### Example 2. Collection of Lipid Transfection Data for Machine Learning

Following lipid synthesis, LNPs composed of the new ionizable lipids, helper lipids (DOPE), PEG-anchored lipids (DMG-PEG2000), and cholesterol were prepared using a classical formulation ratio and loaded with firefly luciferase mRNA (mLuc) described previously,⁹ and thereafter screened in-vitro and in-vivo for mLuc delivery. Hela cells were dosed with the mLuc-LNPs in 96 well plates (100 ng mRNA/well). After overnight incubation, the Bright-Glo Assay was used to assess luciferase expression via luminescence, and transfection data showed a 6% hit rate (FIG. 2C).

To expedite the in vivo screening, a batch-based testing strategy was adopted. First, mLuc-LNPs containing ionizable lipids with the same lipid tail 1 were pooled and intramuscularly injected into mice. After six hours, the transfection potency of each batch was measured via the luminescence signal at the injection site with IVIS imaging (FIGS. 2D-2E), identifying four outstanding tail 1 (ROI>2×10⁸ p/sec/cm²/sr). Batches 1, 2, 3 & 6 (Tail 1 #s 1, 2, 3, and 6) were the top performing batches (FIGS. 2D-2E). Next, among the exceptional mLuc LNPs identified, those carrying ionizable lipids with the same lipid tail 2 were further pooled and intramuscularly injected into mice for the next round of batch-based screening (FIGS. 2F-2G), from which four high-performing combinations of lipid tails 1 and 2 (ROI>5×10⁷ p/sec/cm²/sr) were identified. Batches 1, 2 & 4 (Tail 2 #s 1, 2 & 4) turned out to be the best (FIG. 2F-2G). Subsequently, mLuc LNPs containing these lipid tails were further pooled based on their isocyanide linkers in the third round of batch-based testing (FIGS. 2H-2I). From this final test, eight top-performing combinations of linkers and lipid tails were identified. To generate additional data on optimized ionizable lipids for machine learning training, the set of amine headgroups was expanded from 3 to 25 and combined with the eight top-performing combinations of linkers and lipid tails identified above (FIG. 2J). As shown in FIG. 5, robust in-vivo transfection was observed for many of the lipids.

LNPs composed of these 200 new ionizable lipids with optimized linkers and lipid tails, DOPE, DMG-PEG2000, and cholesterol were formulated with mLuc following the same protocol described above. The transfection efficiency of these mLuc LNPs was then tested in Hela cells (50 ng mRNA/well), as shown in FIG. 2K. In total, the mRNA transfection profiles of 584 ionizable lipids were collected, including both a broader screen of 384 lipids (without optimization) and a narrower screen of 200 lipids (with optimized linkers and tails), which were then used to train the MLA. As in-vivo performance is a good indicator of clinical translation potential, the top performing lipids were selected after in-vivo screening as candidates for re-synthesis, purification and full characterization. In certain cases, lipids that show good delivery of mRNA in-vitro and poor delivery in-vivo were also selected for re-synthesis. In total, 23 top performing lipids were used for re-synthesis and full characterization (FIG. 6).

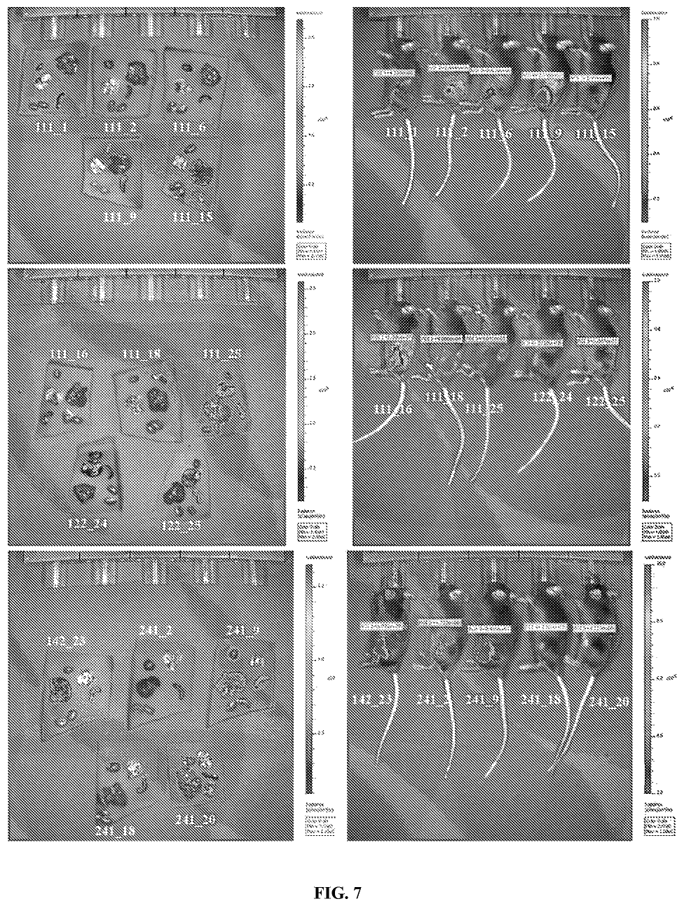

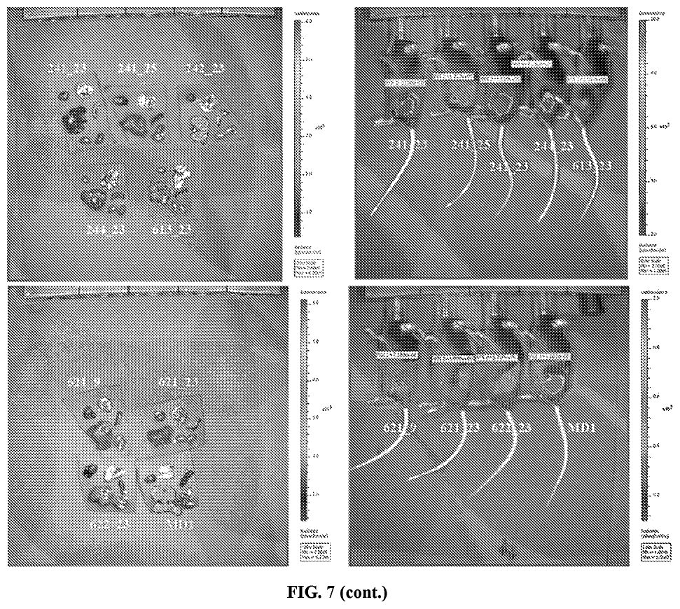

Twenty three re-synthesized and purified lipids were formulated into lipid nanoparticles encapsulating mLuc and administered them to mice by IV and IM. Data obtained after IV administration showed good transfection in different organs, with a number of lipids potently transfecting the liver (FIG. 7, left images). Specifically, 242-23 (with biodegradable tails) potently transfected the liver, and is just one order of magnitude lower than the non-biodegradable MD1 (the most potent liver transfecting lipid known). Lipids such as 241-9 and 142-23 also show potent transfection in the liver. Other organs, such as lungs and spleen were also transfected by some of these lipids. In the lungs, 111-25 and 122-25 showed transfection. Likewise in the spleen, 142-23, 111-25 and 122-25 showed visible level of transfection.

Data from IM administration, which is an indication of ability to deliver mRNA locally, identified 8 lipids to be efficient at local delivery of mRNA with 242-23 also emerging as a high performing lipid (FIG. 7, right images).

Additional linkers shown in FIG. 8A were used to synthesize the lipids shown in FIG. 8B. Linker 5 is a long chain alkyl isocyanide and contains an additional tail. Linkers 6-8 contain a second ionizable amine headgroup. Linker 9 contains an adamantyl isocyanide, and without wishing to be bound by any particular theory, may facilitate hexagonal II (HII) phase formation known to improve transfection performance of ionizable lipids4. Linker 10 contains a pyrazole ring, and without wishing to be bound by any particular theory, may facilitate pi-stacking with nucleobases in nucleic acids.

### Example 3. LNP Screening Guided by Machine Learning

Machine learning (ML) algorithm, a subfield of artificial intelligence, is used to help future decision-making based on past observations. ML techniques are widely adopted in drug development and chemical compound screening,¹⁰ and are leading to a paradigm shift from traditional trial-and-error to artificial intelligence (AI)-based screening. Such methods involve data collection, generation of molecular descriptors, feature cleansing, model training, and validation.

The ability to empirically screen hundreds of lipids for LNP-mediated delivery provides new opportunities to employ machine learning algorithms (MLAs) for LNP design. Ionizable lipid and LNP designs can benefit significantly from MLA due to the complexity and unpredictability of in vivo systems. These model designs hold the potential to improve LNP delivery through large in-silico screens. However, they have not yet been employed in identifying LNPs for mRNA delivery, such as in determining the performance of ionizable lipids for mRNA delivery and thus narrowing down the target lipid candidates from a large library for in vivo testing. It was hypothesized that the guidance of ML modeling would increase the success rate of tested LNPs and thus expedite the lipid screening process.

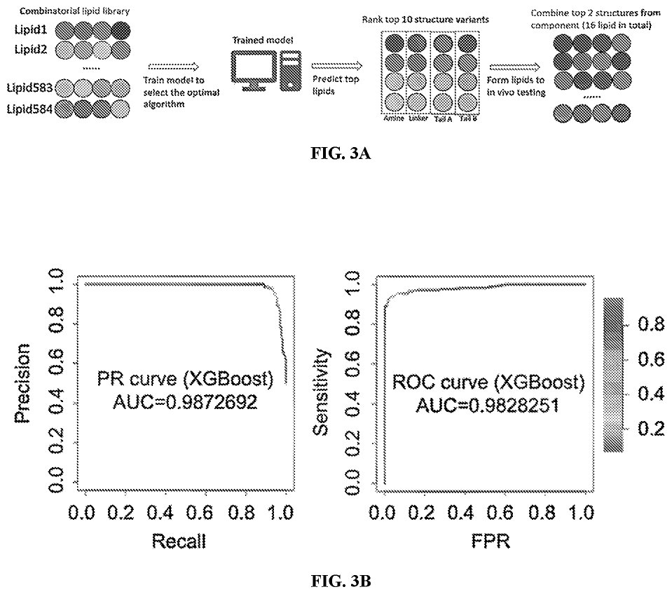

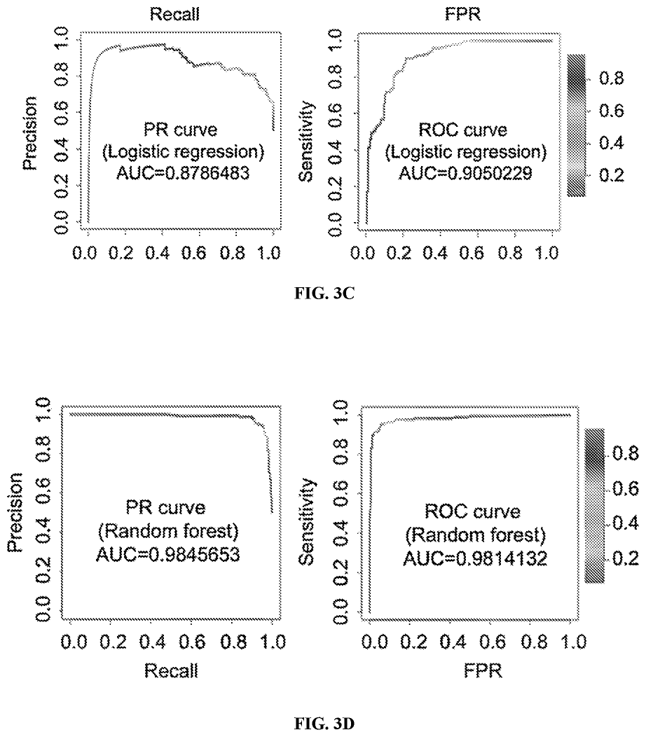

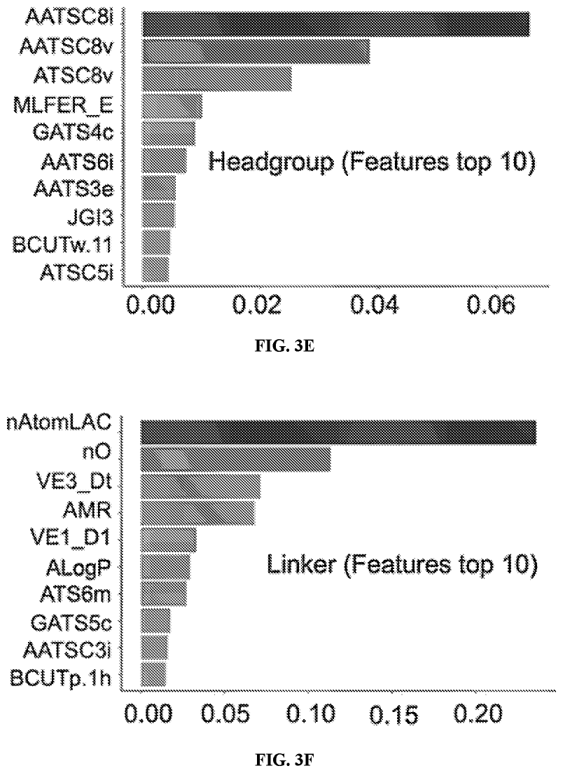

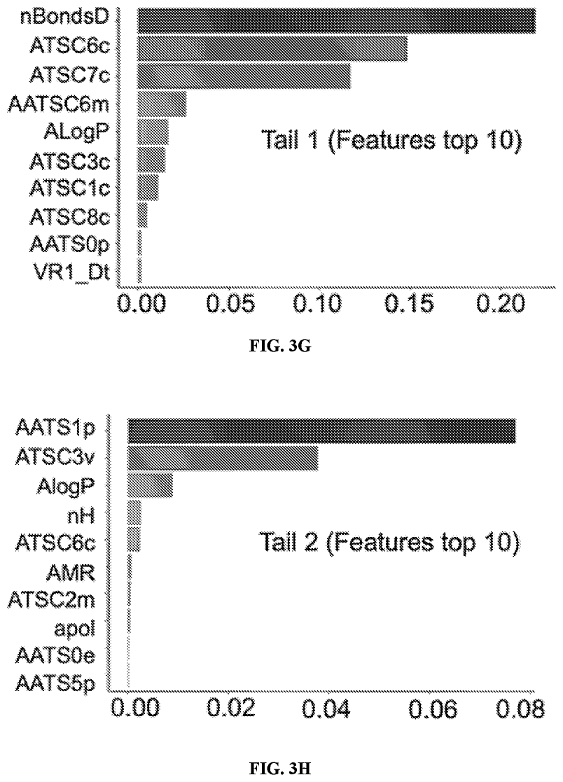

To train an ML algorithm (FIG. 3A), the 584 lipid dataset was transformed into binary outcomes based on the in vitro mLuc transfection results (a positive hit defined as a luminescence signal above 500 RLU). The machine-learning algorithm was then used to select lipids from an in-silico screen of a new library of 40,000 candidate lipids formed by the combination of 40 amines, 10 linkers, 10 tail 1, and 10 tail 2 (FIG. 4A). PaDEL-Descriptor, an open-source software capable of calculating 797 molecular descriptors (663 1D, 2D descriptors, and 134 3D descriptors) and 10 types of fingerprints for chemical compounds¹¹ was used to process the library components. Hence, 807 molecular features (797+10) were calculated for each component (i.e. amine headgroup, linker, lipid tails 1 and 2) of the 4CR lipid library. Therefore, up to 130 million molecular features was obtained from the in silico library of 40,000 lipids (40000×4×807=129,120,000). This number was reduced to a manageable size by applying selection steps, such as removing features with low variance or empty/null values and ensuring feature commonality between the old and new libraries. After this data cleansing, 2014 molecular features were selected from each lipid, forming a design matrix of 8,056,000 (2014×40,000). Due to the relatively low positive hit rate, of about 10%, in the binary training dataset, it is important to resample the imbalanced data to avoid potential bias of the ML algorithm.

Next, three nonlinear machine learning algorithms (random forest, logistic regression, gradient boosting) were evaluated for their interpretability and ability to learn from the dataset. After tuning the hyperparameter by randomized search, Gradient Boosting (XGBoost) reached the highest receiver operating characteristic area under the curve (ROC-AUC) of 0.983 and precision-recall area under the curve (PR-AUC) of 0.987 among the three models (FIGS. 3B-3D). Therefore, XGBoost was selected as the final model for prediction, as it achieved the highest quality distinction between positive and negative classes.

XGBoost may generate unique predictions for each run due to its stochastic learning feature. To mitigate the difference, the model was re-run with 1,000 random seeds from which high-value lipids were predicted among the in silico library of 40,000 lipid candidates. The results from 1,000 runs were then aggregated by selecting the top fifty lipids in each run, leading to a collection of 50,000 top lipid candidates (counting duplicates). For each of the four components (headgroup, linker and both tails), the most commonly successful structures were determined. Additionally, chemical descriptors for each of the four components were noted based on the significance of their contribution to mRNA transfection potency (FIGS. 3E-3H).

Based on this information, the top 2 structures were chosen from each component, and 16 new lipids (2 head groups×2 linkers×2 tail A×2 tail B) were synthesized (FIGS. 4B-4C). LNPs containing these ionizable lipids were formulated with DOPE, cholesterol, DMG-PEG2000, and mLuc, and then individually tested in mice (0.1 mg/kg). At six-hours post-intramuscular injection, the transfection potency of each LNP was measured via the luminescent signal at the injection site with IVIS imaging. As shown in FIG. 4D, 5 out of 16 lipids showed a transfection potency greater than or equal to the benchmark LNP composed of MC3. Intramuscular transfection by the 119-23 (FIG. 4E) formulated LNP was two orders of magnitude higher than that of the MC3 formulated LNP. While the traditional trial-and-error screening method often leads to a positive hit rate of less than 2%, the sixteen lipids synthesized based on the MLA-aided design resulted in 31% of lipids whose LNP provided high in vivo efficacy (>10⁷ p/sec/cm²/sr), highlighting the ability of the model to screen for clinically relevant lipids. This approach not only accelerates the identification of new LNPs which satisfy diverse delivery tasks but also enables the exploration of vast chemical spaces in combinatorial lipid libraries beyond the reach of current experimental approaches.

### Example 4. Further Studies Using Lipids 119-23 and 242-23

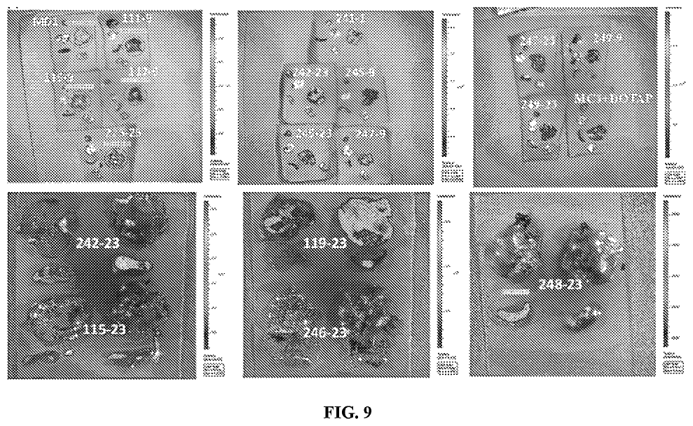

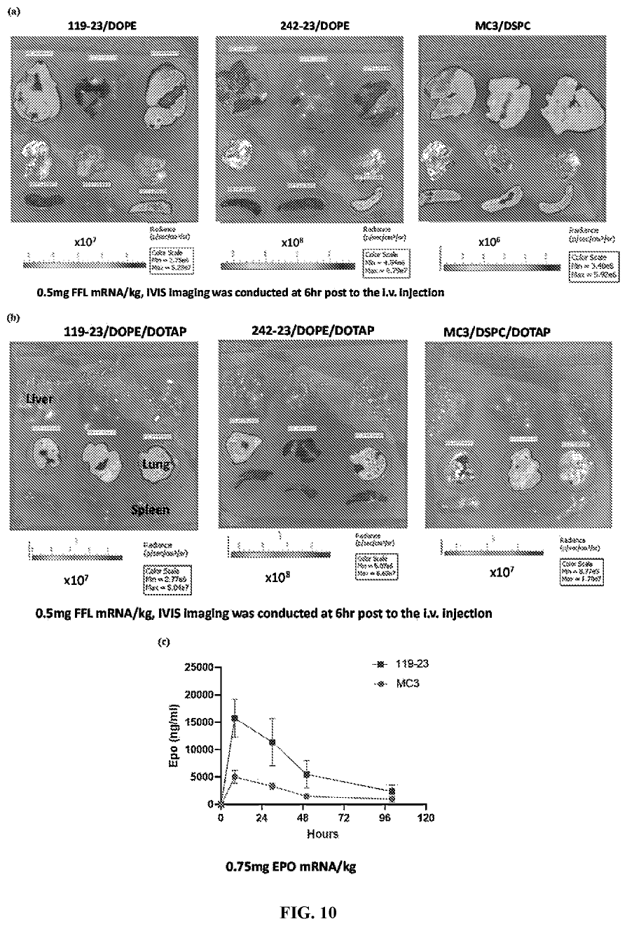

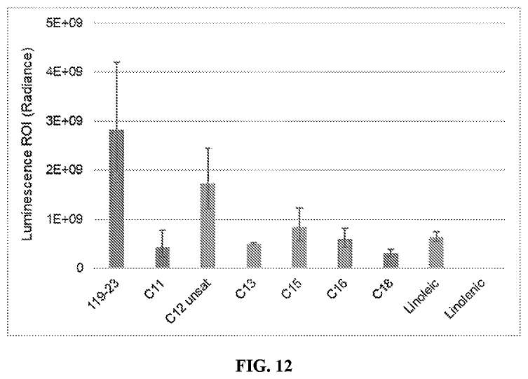

To investigate the potential organ-tropic properties of lipid 119-23, the above mLuc 119-23 LNP was administered into mice via intravenous injection (0.25 mg/kg). As shown in FIG. 4F, the mRNA transfection potency of 119-23 LNP in the liver was about 5-fold higher than MC3, and 3-fold higher than C12-200, two non-biodegradable ionizable lipid benchmarks both known for their high RNA transfection capability in the liver¹². Moreover, the biodegradability of 119-23 (imparted by ester bonds) may widen the therapeutic window of mRNA LNPs due to reduced toxicity and side-effects. A head-to-head comparison of lipids 119-23 and 242-23 was also carried out, comparing with MC3, an ionizable lipid in US FDA approved Patisiran (FIG. 9). Both 119-23 and 242-23 performed much better than MC3 at transfecting the liver and spleen, with 119-23 being the better of the two at transfecting the liver (FIG. 10A). As an attempt to further improve the transfection efficacy of 119-23, eight structural analogs of the lipid were synthesized, in which the two tails were systematically varied (FIG. 11). However, none of these analogs performed better than 119-23 after intramuscular injection of LNP encapsulating mLuc (FIG. 12).

The 119-23 LNP formulation led to a strong mRNA transfection in the spleen following intravenous injection, with a luminescence signal 8-fold improved over that of C12-200. To further interrogate the transfection of 119-23 in other main organs, MC3, C12-200 and 119-23 LNPs were navigated to the lung by adding DOTAP, a cationic lipid as the fifth composition to their formulations following the “SORT” method,¹³ in which LNPs are doped with charged molecules to facilitate their selective targeting to specific organs. After intravenous administration, DOTAP-containing 119-23 LNP displayed potent transfection in the lung as shown by 6-fold and 30-fold improved luminescence signal compared to an equivalent C12-200 and MC3 formulation respectively (FIG. 4G). Similar LNPs made from 242-23 performed better than MC3 at transfecting the lungs using SORT (FIG. 10B). These results suggest the ability of ionizable lipids 119-23 and 242-23 to target multiple tissues, as well as potential utility for distinct RNA delivery tasks, with excellent potency for immune cell transfection and specificity for the liver, spleen, or lungs depending on helper lipid formulation. This high throughput data- and machine learning-driven platform may facilitate the development of robust and widely applicable platforms for RNA delivery.

To demonstrate that the lipids disclosed herein will work with other mRNA for therapeutic purposes, lipid 119-23 was used for liver transfection to formulate LNP encapsulating erythropoietin (EPO) mRNA, and was compared to MC3 after IV administration. Again, 119-23 was about 3-folds better than MC3 at the peak level of expression and all through the time course of the study (FIG. 10C).

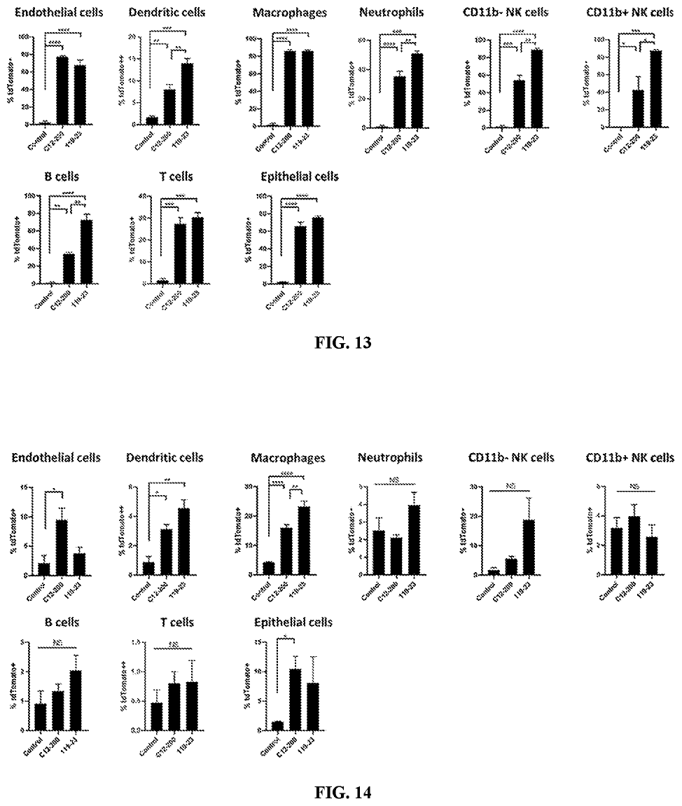

Important for the therapeutic success of RNA delivery by LNPs is the ability to target delivery to specific cell types within the various organs. For instance, some protein therapies may involve mRNA delivery to epithelial cells, as in the case of lung disease¹⁴. Vaccines and immunotherapeutic applications involve immune, and especially dendritic cell targeting¹⁵. To determine cell types transfected by 119-23 LNPs in various organs, LNPs containing CRE mRNA (0.75 mg/kg) were administered intravenously to Ail4 reporter mice (Jackson Labs, #7914). Ail4 reporter mice encode a loxP-flanked STOP cassette preventing transcription of the tdTomato fluorophore; successful translation of CRE mRNA produces tdTomato⁺ cells, which are identifiable and quantifiable with flow cytometry (FIG. 4H). Two days following LNP administration, spleen and liver tissues were processed into single-cell suspensions and stained for various endothelial, epithelial, and immune cell lineages. The mRNA transfection in different cell phenotypes was quantified by flow cytometry as the percentage of cells expressing tdTomato. While liver endothelial and epithelial cells were transfected by 119-23 and C12-200 formulated LNPs at comparable levels, 119-23 LNPs could transfect a wide range of both innate and adaptive immune cells, with delivery to dendritic cells (14%), neutrophils (51%), natural killer cells (89%), and B cells (72%) significantly exceeding that observed with C12-200 LNPs (FIGS. 4I, 13). In the spleen, significant transfection was observed in dendritic cells (4.6%) and macrophages (23%) by 119-23 LNPs, with delivery to macrophages exceeding that achieved by C12-200 LNPs by 5% (FIGS. 4J, 14).

To assess delivery to various cells in the lung, Ai14 mice were dosed intravenously with lung targeted 119-23 LNPs (0.75 mg/kg CRE mRNA intravenously, as above). Flow cytometry data showed significant mRNA transfection in lung endothelial cells (20%), epithelial cells (11%), and immune cells (11%) (FIG. 4K). Together these data suggest that lipid 119-23, and others identified by ML-guided screening, may be useful for the efficient transfection and translation of functional mRNA in a wide range of endothelial, epithelial, and immune cell types in various organs.

### Example 5. Results

By employing ML-guided methodologies to screen vast chemical libraries compatible with the 4CR reaction system, lipid 119-23 was identified as a promising candidate for mRNA delivery to specific endothelial, epithelial, and immune cell types in multiple organs (liver, spleen, and lung). Intracellular mRNA delivery, and ultimately translation of functional protein, in the intended cell type is important for therapeutic success. However, systemic, extrahepatic delivery of RNA remains difficult. In particular, there is substantial interest in developing novel lipids for mRNA delivery to immune cells, antigen presentation in vaccines, and effector functions in antiviral and cancer immunotherapy applications. Lipid 119-23, identified here, demonstrated functional CRE mRNA delivery to multiple liver immune cells, including antigen-presenting cells (APCs), dendritic cells, and effectors, natural killer cells, at levels that exceeded those observed with the benchmark lipid C12-200 (FIGS. 4I and 13).

Further, delivery to splenic APCs, macrophages exceeded that achieved by C12-200 LNPs by 5% (FIGS. 4J and 14). This data is consistent with other literature which employed DNA barcoding-based analytical methods to screen adamantyl-containing phospholipids to target liver immune cells¹⁶. DNA-barcoding identified lipids that preferentially targeted liver macrophages over hepatocytes. Other strategies to increase RNA delivery to immune cells, including design-of-experiment methodologies to optimize LNP formulation and the collection of large data sets to elucidate lipid structure-function relationships, have previously been reported^{5, 17, 18}. Lipid 119-23, identified by ML-guided screening, allowed functional mRNA delivery to a wide range of liver, spleen, and lung immune cells. The ability to efficiently deliver mRNA to both APCs and effector immune cells, including natural killer and some T cells, may pave the way for new, clinically-relevant, mRNA-based therapies. This high throughput data- and machine learning-driven platform may further allow for the development of new RNA therapeutics and translational strategies.

### Example 6. Experimental Procedures for Synthesis of Ionizable Lipids

All solvents and reagents used were obtained commercially and used as such unless noted otherwise. ¹H and ¹³C NMR spectra were recorded in CDCl₃ at room temperature using a Bruker Ultrashield 400 MHz instrument. Spectra were processed with Chemical shifts reported as parts per million (ppm) relative to TMS (0.00) for ¹H. Silica gel chromatography was performed on ISCO CombiFlash Rf+ Lumen Instruments using ISCO RediSep Rf Gold Flash Cartridges (particle size: 20-40 microns). All final compounds were confirmed by mass spectrometry using direct injection method on QTOF-HRMS (Agilent) coupled to an Agilent Infinity 1260 LC system. Only top performing lipids after screening by batch analysis were fully characterized by ¹H and ¹³C NMR.

### General Synthesis of Tail 1

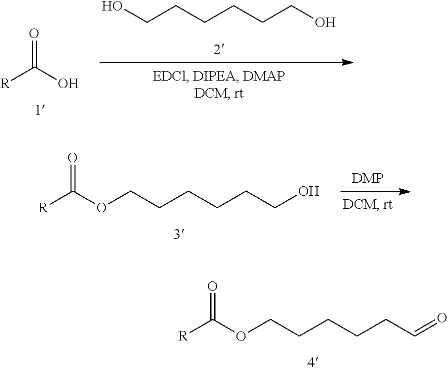

### Synthesis of 6-hydroxyhexyl oleate, 3′

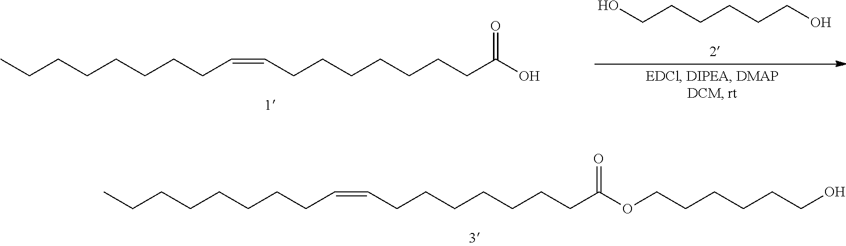 [SMILES: OCCCCCCO ; CCCCCCCC/C=C\CCCCCCCC(=O)O ; CCCCCCCC/C=C\CCCCCCCC(=O)OCCCCCCO]

Oleic acid (5 g, 17.7 mmol), N-(3-dimethylaminopropyl)-N′-ethylcarbodiimide hydrochloride (EDC HCl, 5.1 g, 26.6 mmol), hexane-1,6-diol (10.5 g, 88.5 mmol), 4-(dimethylamino) pyridine (DMAP, 1.1 g, 8.9 mmol) and N, N-diisopropylethylamine (DIPEA, 6.2 ml, 35.4 mmol) were dissolved in dichloromethane (200 ml). The reaction was stirred at room temperature under nitrogen for 18 h, then washed with a saturated aqueous sodium bicarbonate solution. The organic layer was separated, washed with brine, dried over Na₂SO₄, filtered, and the filtrate was evaporated under vacuum. The residue was purified by silica gel chromatography (0-50% ethyl acetate in hexanes) to give 6-hydroxyhexyl oleate, 3′ (5.8 g, 15.2 mmol, 86%) as a colorless oil. ¹H NMR (400 MHz, CDCl₃) δ 5.43-5.29 (m, 2H), 4.09 (t, J=6.7 Hz, 2H), 3.67 (t, J=6.5 Hz, 2H), 2.31 (t, J=7.5 Hz, 2H), 2.03 (q, J=6.5 Hz, 4H), 1.73-1.53 (m, 6H), 1.49-1.17 (m, 24H), 0.95-0.83 (m, 3H).

### Synthesis of 6-oxohexyl oleate, 4′

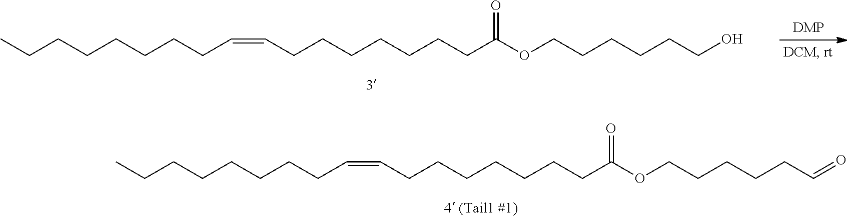 [SMILES: CCCCCCCC/C=C\CCCCCCCC(=O)OCCCCCCO ; CCCCCCCC/C=C\CCCCCCCC(=O)OCCCCCC=O]

6-hydroxyhexyl oleate, 10 (5.8 g, 15.2 mmol) was dissolved in dichloromethane 300 ml followed by Dess-Martin Periodinane (9.6 g, 22.8 mmol). The reaction was stirred under nitrogen at room temperature for 2 h. After confirmation of reaction completion by thin layer chromatography, sodium thiosulfate pentahydrate (50% w/v, 200 ml) was added to the reaction and left to stir for additional 15 mins. Organic layer was thereafter separated, washed with brine, dried over Na₂SO₄, filtered, and the filtrate was evaporated under vacuum. The residue was purified by silica gel chromatography (0-50% ethyl acetate in hexanes) to give 6-oxohexyl oleate, 4′ (3.5 g, 9.2 mmol, 61%) as a colorless oil. ¹H NMR (400 MHz, CDCl₃) δ 9.79 (s, 1H), 5.42-5.33 (m, 2H), 4.09 (t, J=6.6 Hz, 2H), 2.47 (td, J=7.3, 1.7 Hz, 2H), 2.31 (t, J=7.5 Hz, 2H), 2.03 (q, J=6.5 Hz, 4H), 1.67 (dp, J=15.1, 7.8 Hz, 7H), 1.48-1.21 (m, 23H), 0.90 (t, J=6.8 Hz, 3H).

### Synthesis of 6-hydroxyhexyl (9Z,12Z)-octadeca-9,12-dienoate, 3′

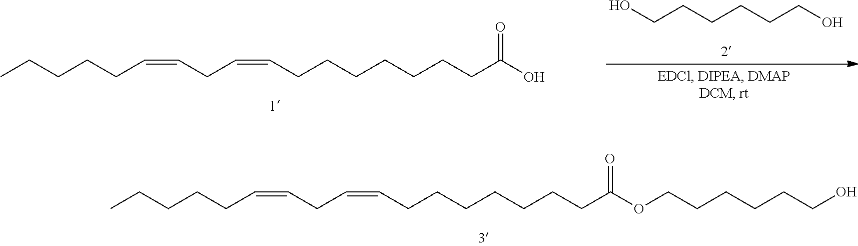 [SMILES: OCCCCCCO ; CCCCC/C=C\C/C=C\CCCCCCCC(=O)O ; CCCCC/C=C\C/C=C\CCCCCCCC(=O)OCCCCCCO]

6-hydroxyhexyl (9Z,12Z)-octadeca-9,12-dienoate, 3′ (linoleic acid) was made using synthesis of 6-hydroxyhexyl oleate, 3′ above. ¹H NMR (500 MHz, CDCl₃) δ 5.47-5.26 (m, 4H), 4.09 (t, J=6.7 Hz, 2H), 3.67 (t, J=6.5 Hz, 2H), 2.80 (t, J=6.7 Hz, 2H), 2.31 (t, J=7.6 Hz, 2H), 2.07 (q, J=7.0 Hz, 4H), 1.74-1.53 (m, 7H), 1.49-1.18 (m, 18H), 0.91 (t, J=6.8 Hz, 3H).

### Synthesis of 6-oxohexyl (9Z,12Z)-octadeca-9,12-dienoate, 4′

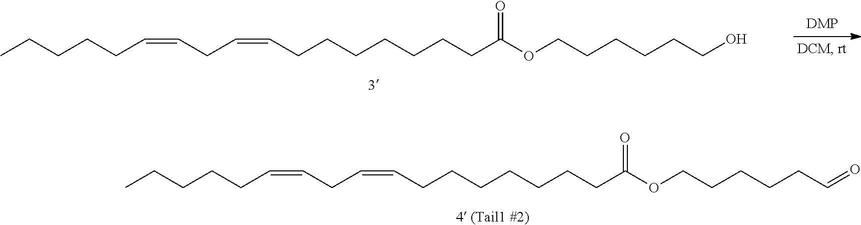 [SMILES: CCCCC/C=C\C/C=C\CCCCCCCC(=O)OCCCCCCO ; CCCCC/C=C\C/C=C\CCCCCCCC(=O)OCCCCCC=O]

6-oxohexyl (9Z,12Z)-octadeca-9,12-dienoate, 4′ (linoleic acid) was made using synthesis of 6-oxohexyl oleate, 4′ above. ¹H NMR (400 MHz, CDCl₃) δ 9.79 (s, 1H), 5.59-5.15 (m, 4H), 4.09 (t, J=6.6 Hz, 2H), 2.79 (t, J=6.4 Hz, 2H), 2.31 (t, J=7.6 Hz, 2H), 2.06 (dd, J=8.1, 5.7 Hz, 4H), 1.68 (dq, J=14.4, 7.2 Hz, 8H), 1.44-1.22 (m, 16H), 0.91 (t, J=6.7 Hz, 3H).

### Synthesis of 6-hydroxyhexyl stearate, 3′

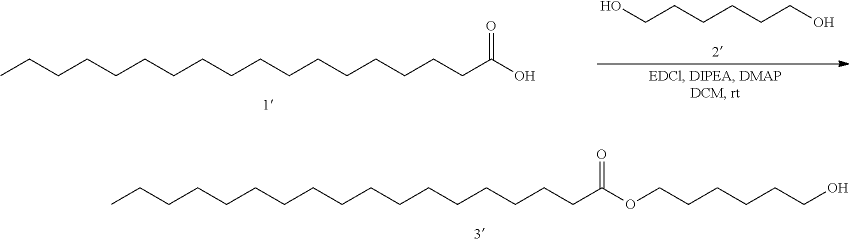 [SMILES: OCCCCCCO ; CCCCCCCCCCCCCCCCCC(=O)O ; CCCCCCCCCCCCCCCCCC(=O)OCCCCCCO]

6-hydroxyhexyl stearate 3′ (octadecanoic acid) was made using synthesis of 6-hydroxyhexyl oleate, 3′ above. ¹H NMR (500 MHz, CDCl₃) δ 4.09 (t, J=6.7 Hz, 2H), 3.67 (t, J=6.6 Hz, 2H), 2.31 (t, J=7.6 Hz, 2H), 1.64 (ddt, J=22.0, 14.5, 7.1 Hz, 7H), 1.42 (dq, J=6.6, 2.9 Hz, 4H), 1.28 (d, J=2.5 Hz, 27H), 0.90 (t, J=6.9 Hz, 3H).

### Synthesis of 6-oxohexyl stearate, 4′

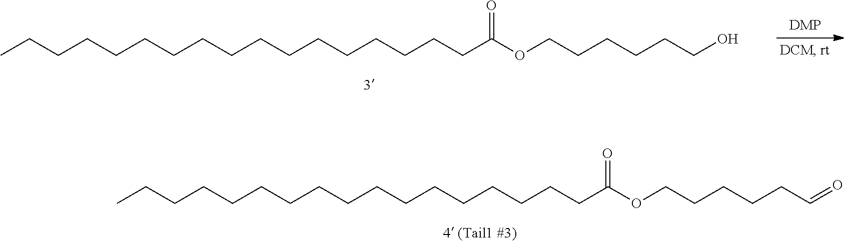 [SMILES: CCCCCCCCCCCCCCCCCC(=O)OCCCCCCO ; CCCCCCCCCCCCCCCCCC(=O)OCCCCCC=O]

6-oxohexyl stearate 4′ (octadecanoic acid) was made using synthesis of 6-oxohexyl oleate, 4′ above. ¹H NMR (400 MHz, CDCl₃) δ 9.80 (s, 1H), 4.09 (t, J=6.6 Hz, 2H), 2.48 (td, J=7.3, 1.7 Hz, 2H), 2.31 (t, J=7.6 Hz, 2H), 1.77-1.57 (m, 7H), 1.50-1.36 (m, 2H), 1.28 (s, 27H), 0.90 (t, J=6.8 Hz, 3H).

### Synthesis of 6-hydroxyhexyl palmitate, 3′

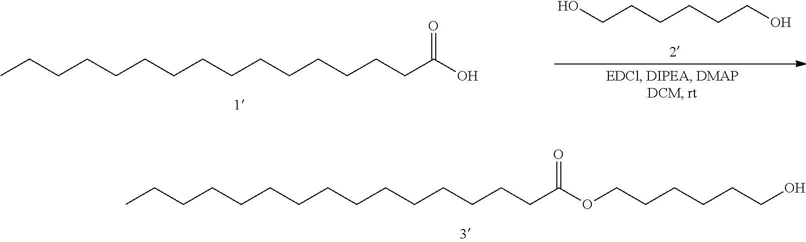 [SMILES: OCCCCCCO ; CCCCCCCCCCCCCCCC(=O)O ; CCCCCCCCCCCCCCCC(=O)OCCCCCCO]

6-hydroxyhexyl palmitate, 3′ was made using synthesis of 6-hydroxyhexyl oleate, 3′ above. ¹H NMR (500 MHz, CDCl₃) δ 4.09 (t, J=6.7 Hz, 2H), 3.67 (q, J=6.2 Hz, 2H), 2.31 (t, J=7.6 Hz, 2H), 1.64 (tp, J=21.2, 6.7 Hz, 7H), 1.48-1.36 (m, J=3.6, 3.0 Hz, 4H), 1.36-1.20 (m, 22H), 0.90 (t, J=6.9 Hz, 3H).

### Synthesis of 6-oxohexyl palmitate, 4′

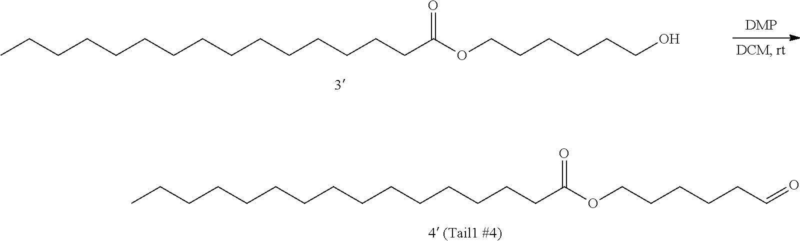 [SMILES: CCCCCCCCCCCCCCCC(=O)OCCCCCCO ; CCCCCCCCCCCCCCCC(=O)OCCCCCC=O]

6-oxohexyl palmitate, 4′ was made using synthesis of 6-oxohexyl oleate, 4′ above. ¹H NMR (500 MHz, CDCl₃) δ 9.79 (s, 1H), 4.09 (t, J=6.6 Hz, 2H), 2.48 (td, J=7.3, 1.7 Hz, 2H), 2.31 (t, J=7.5 Hz, 2H), 1.75-1.53 (m, 7H), 1.28 (s, 24H), 0.90 (t, J=6.9 Hz, 3H).

### Synthesis of 6-hydroxyhexyl tridecanoate, 3′

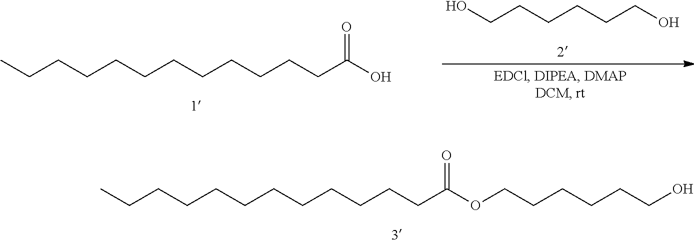 [SMILES: OCCCCCCO ; CCCCCCCCCCCCC(=O)O ; CCCCCCCCCCCCC(=O)OCCCCCCO]

6-hydroxyhexyl tridecanoate, 4′ was made using synthesis of 6-hydroxyhexyl oleate, 3′ above. ¹H NMR (400 MHz, CDCl₃) δ 4.08 (t, J=6.7 Hz, 2H), 3.66 (t, J=6.6 Hz, 2H), 2.30 (t, J=7.5 Hz, 2H), 1.73-1.55 (m, 7H), 1.41 (p, J=3.6 Hz, 5H), 1.29 (d, J=10.3 Hz, 18H), 0.90 (t, J=6.7 Hz, 3H).

### Synthesis of 6-oxohexyl tridecanoate, 4′

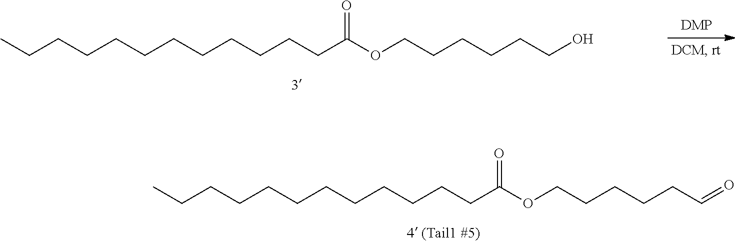 [SMILES: CCCCCCCCCCCCC(=O)OCCCCCCO ; CCCCCCCCCCCCC(=O)OCCCCCC=O]

6-oxohexyl tridecanoate, 4′ (tridecanoic acid) was made using synthesis of 6-oxohexyl oleate, 4′ above. ¹H NMR (400 MHz, CDCl₃) δ 9.79 (s, 1H), 4.09 (t, J=6.6 Hz, 2H), 2.47 (td, J=7.3, 1.7 Hz, 2H), 2.31 (t, J=7.6 Hz, 2H), 1.76-1.55 (m, 6H), 1.51-1.38 (m, 2H), 1.29 (d, J=10.1 Hz, 18H), 0.90 (t, J=6.8 Hz, 3H).

### Synthesis of 6-hydroxyhexyl undecanoate, 3′

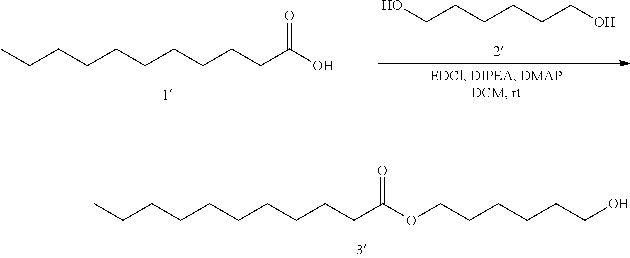 [SMILES: CCCCCCCCCCC(=O)O ; OCCCCCCO ; CCCCCCCCCCC(=O)OCCCCCCO]

6-hydroxyhexyl undecanoate, 3′ was made using synthesis of 6-hydroxyhexyl oleate, 3′ above. ¹H NMR (500 MHz, CDCl₃) δ 4.09 (t, J=6.7 Hz, 2H), 3.67 (t, J=6.6 Hz, 2H), 2.31 (t, J=7.5 Hz, 2H), 1.64 (dp, J=22.4, 7.0 Hz, 6H), 1.42 (dq, J=6.7, 2.9 Hz, 5H), 1.36-1.21 (m, 14H), 0.90 (t, J=6.9 Hz, 3H).

### Synthesis of 6-oxohexyl undecanoate, 4′

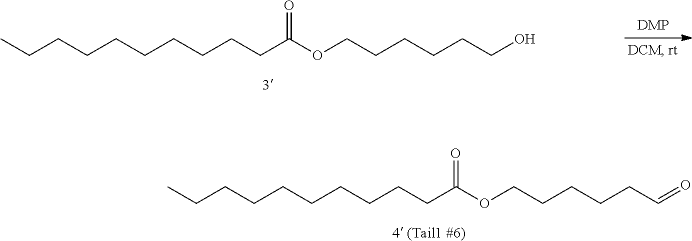 [SMILES: CCCCCCCCCCC(=O)OCCCCCCO ; CCCCCCCCCCC(=O)OCCCCCC=O]

6-oxohexyl undecanoate, 4′ was made using synthesis of 6-oxohexyl oleate, 4′ above.

### Synthesis of 6-hydroxyhexyl nonanoate, 3′

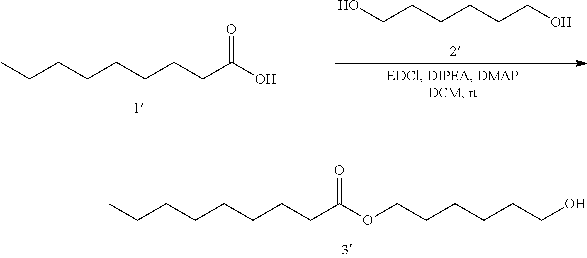 [SMILES: OCCCCCCO ; CCCCCCCCC(=O)O ; CCCCCCCCC(=O)OCCCCCCO]

6-hydroxyhexyl nonanoate, 3′ (nonanoic acid) was made using synthesis of 6-hydroxyhexyl oleate, 3′ above. ¹H NMR (400 MHz, CDCl₃) δ 4.09 (t, J=6.7 Hz, 2H), 3.67 (t, J=6.5 Hz, 2H), 2.31 (t, J=7.6 Hz, 2H), 1.72-1.54 (m, 6H), 1.41 (p, J=3.5 Hz, 5H), 1.37-1.20 (m, 10H), 0.90 (t, J=6.7 Hz, 3H).

### Synthesis of 6-oxohexyl nonanoate, 4′

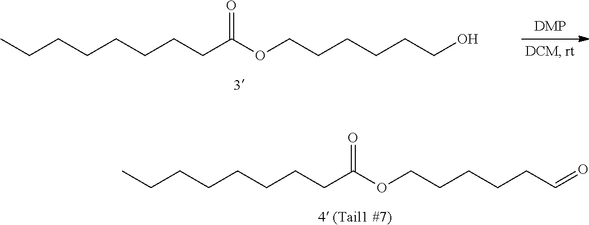 [SMILES: CCCCCCCCC(=O)OCCCCCCO ; CCCCCCCCC(=O)OCCCCCC=O]

6-oxohexyl nonanoate, 4′ was made using synthesis of 6-oxohexyl oleate, 4′ above. ¹H NMR (500 MHz, CDCl₃) δ 9.79 (s, 1H), 4.09 (t, J=6.6 Hz, 2H), 2.47 (td, J=7.3, 1.7 Hz, 2H), 2.31 (t, J=7.6 Hz, 2H), 1.75-1.57 (m, 7H), 1.46-1.37 (m, 2H), 1.34-1.23 (m, 9H), 0.90 (t, J=6.9 Hz, 3H).

### Synthesis of Tail1 #8

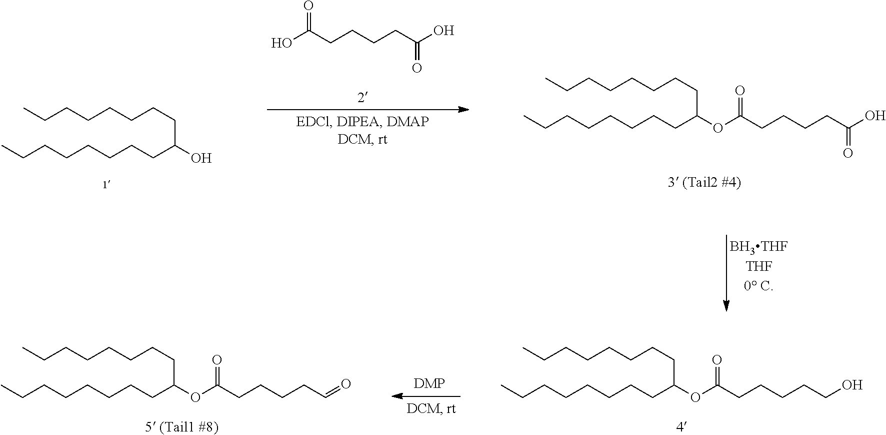 [SMILES: O=C(O)CCCCC(=O)O ; CCCCCCCCC(O)CCCCCCCC ; CCCCCCCCC(CCCCCCCC)OC(=O)CCCCC(=O)O ; CCCCCCCCC(CCCCCCCC)OC(=O)CCCCC=O ; CCCCCCCCC(CCCCCCCC)OC(=O)CCCCCO]

### Synthesis of 6-(heptadecan-9-yloxy)-6-oxohexanoic acid, 3′ (Tail2 #4)

9-heptadecanol (3.0 g, 11.7 mmol), N-(3-dimethylaminopropyl)-N′-ethylcarbodiimide hydrochloride (EDC HCl, 4.5 g, 23.4 mmol), adipic acid (3.4 g, 23.2 mmol), 4-(dimethylamino) pyridine (DMAP, 1.5 g, 11.7 mmol) and N, N-diisopropylethylamine (DIPEA, 4.1 ml, 23.4 mmol) were dissolved in dichloromethane (300 ml). The reaction was stirred at room temperature under nitrogen for 18 h. Dichloromethane was evaporated under vacuum, and the residue purified by silica gel chromatography (10% methanol in dichloromethane) to give 6-(heptadecan-9-yloxy)-6-oxohexanoic acid, 3′ (2.3 g, 6.0 mmol, 51%) as a colorless oil. ¹H NMR (500 MHz, CDCl₃) δ 4.89 (p, J=6.3 Hz, 1H), 2.41 (h, J=3.6, 3.2 Hz, 2H), 2.37-2.29 (m, 2H), 1.71 (h, J=4.0, 3.5 Hz, 4H), 1.58 (s, 4H), 1.28 (d, J=6.8 Hz, 24H), 0.90 (t, J=6.9 Hz, 6H).

### Synthesis of heptadecan-9-yl 6-hydroxyhexanoate, 4′

6-(heptadecan-9-yloxy)-6-oxohexanoic acid, 3′ (2 g, 5.2 mmol) was dissolved in THF (20 ml) and cooled to 0° C. while stirring under nitrogen. BHF·THF (10.5 ml, 10.5 mmol) was added dropwise to the reaction, after which the reaction was left to stir at 0° C. under nitrogen for 3 hr. The reaction was quenched by adding brine in drops until gas evolution ceased. The mixture was then extracted with ethyl acetate (100 ml). Organic layer was dried over sodium sulfate, filtered, and the organic solvent was evaporated under vacuum. Crude residue was purified by silica gel chromatography (0-50% ethyl acetate in hexanes) to give heptadecan-9-yl 6-hydroxyhexanoate 4′ (1.5 g, 4.0 mmol, 77%) as a colorless oil. ¹H NMR (500 MHz, CDCl₃) δ 4.89 (p, J=6.3 Hz, 1H), 3.68 (t, J=6.6 Hz, 2H), 2.33 (t, J=7.5 Hz, 2H), 1.74-1.57 (m, 4H), 1.53 (q, J=6.2 Hz, 4H), 1.48-1.37 (m, 4H), 1.29 (d, J=8.5 Hz, 23H), 0.90 (t, J=6.8 Hz, 6H).

### Synthesis of heptadecan-9-yl 6-oxohexanoate, 5′ (Tail1 #8)

Heptadecan-9-yl 6-hydroxyhexanoate, 4′ (1.4 g, 3.8 mmol) was dissolved in dichloromethane 100 ml followed by Dess-Martin Periodinane (2.4 g, 5.7 mmol). The reaction was stirred under nitrogen at room temperature for 2 h. After confirmation of reaction completion by thin layer chromatography, sodium thiosulfate pentahydrate (50% w/v, 200 ml) was added to the reaction and left to stir for additional 15 mins. Organic layer was thereafter separated, washed with brine, dried over Na₂SO₄, filtered, and the filtrate was evaporated under vacuum. The residue was purified by silica gel chromatography (0-50% ethyl acetate in hexanes) to give heptadecan-9-yl 6-oxohexanoate, 5′ (0.8 g, 2.2 mmol, 56%) as a light brown oil. ¹H NMR (500 MHz, CDCl₃) δ 9.79 (s, 1H), 4.89 (p, J=6.3 Hz, 1H), 2.49 (tt, J=6.9, 1.7 Hz, 2H), 2.34 (dd, J=10.5, 3.2 Hz, 2H), 1.70 (h, J=4.3, 3.7 Hz, 4H), 1.64-1.42 (m, 5H), 1.28 (d, J=6.0 Hz, 23H), 0.90 (t, J=6.8 Hz, 6H).

### Synthesis of Lipids Using Ugi 4-Component Reaction

### Synthesis of 242-23

A solution of 6-oxohexyl (9Z,12Z)-octadeca-9,12-dienoate (0.1 g, 0.3 mmol) and N¹, N¹-dimethylpropane-1,3-diamine (0.03 g, 0.3 mmol) in methanol (1 ml) in a glass vial was stirred at room temperature for 1 hr, after which tert-butyl isocyanide (0.02 g, 0.3 mmol) and 6-(heptadecan-9-yloxy)-6-oxohexanoic acid (0.1 g, 0.3 mmol) were added. The reaction was left to stir at room temperature overnight. The reaction mixture was purified by silica gel chromatography (10% methanol in dichloromethane with 0.1% ammonium hydroxide) to give 242-23 as a light brown oil (0.16 g, 0.2 mmol, 67%). QTOF MS (ESI): m/z calcd for C₅₉H₁₁₀N₃₀O₈⁺ (M+H), 988.82929; found, 930.8253. ¹H NMR (500 MHz, CDCl₃) δ 7.04 (t, J=5.6 Hz, 1H), 5.46-5.29 (m, 4H), 4.84 (dt, J=32.7, 7.0 Hz, 2H), 4.05 (t, J=6.7 Hz, 2H), 3.93 (dd, J=17.9, 6.1 Hz, 1H), 3.79 (dd, J=18.0, 5.1 Hz, 1H), 3.35 (td, J=9.3, 5.9 Hz, 2H), 2.79 (t, J=6.7 Hz, 2H), 2.45 (t, J=7.0 Hz, 3H), 2.40-2.16 (m, 11H), 2.13-1.97 (m, 6H), 1.86-1.57 (m, 12H), 1.46 (s, 13H), 1.43-1.16 (m, 41H), 0.90 (q, J=6.7 Hz, 9H). ¹³C NMR (126 MHz, CDCl₃) δ 174.52, 173.91, 173.22, 171.76, 168.53, 130.21, 130.07, 128.03, 127.92, 81.83, 74.33, 64.12, 41.81, 34.37, 34.34, 34.10, 33.21, 31.86, 31.53, 29.62, 29.55, 29.50, 29.48, 29.35, 29.27, 29.25, 29.19, 29.16, 29.14, 28.52, 28.05, 27.96, 27.20, 26.05, 25.83, 25.63, 25.33, 24.98, 24.91, 24.79, 22.66, 22.58, 14.11, 14.08.

### Synthesis of 111-6

Following the same procedure described for compound 242-23 above, compound 111-6 was made using 6-oxohexyl oleate (0.05 g, 0.13 mmol), 3-(1H-imidazol-1-yl) propan-1-amine (0.02 g, 0.13 mmol), oleic acid (0.04 g, 0.13 mmol), and tert-butyl 2-isocyanoacetate (0.02 g, 0.13 mmol) in methanol (1 ml) to give compound 111-6 as a light brown oil (0.07 g, 0.08 mmol, 62%). QTOF MS (ESI): m/z calcd for C₅₅H₉₉N₃O₆⁺ (M+H), 911.75646; found, 911.7564. ¹H NMR (400 MHz, CDCl₃) δ 7.57 (s, 1H), 7.09 (d, J=7.3 Hz, 2H), 6.98 (d, J=8.8 Hz, 1H), 5.43-5.29 (m, 4H), 4.83 (t, J=7.6 Hz, 1H), 4.10-3.86 (m, 5H), 3.73 (dd, J=17.9, 5.1 Hz, 1H), 3.27 (td, J=9.4, 5.8 Hz, 2H), 2.29 (t, J=7.6 Hz, 3H), 2.17 (td, J=7.2, 3.5 Hz, 3H), 2.03 (dq, J=12.8, 6.2 Hz, 10H), 1.61 (p, J=7.0 Hz, 8H), 1.46 (d, J=12.5 Hz, 11H), 1.41-1.18 (m, 42H), 0.89 (t, J=6.7 Hz, 6H). ¹³C NMR (101 MHz, CDCl₃) δ 174.87, 173.93, 171.77, 168.52, 137.09, 130.00, 129.75, 129.72, 129.62, 118.58, 81.97, 64.00, 60.39, 44.40, 42.20, 41.71, 34.33, 33.49, 31.90, 30.59, 29.76, 29.70, 29.70, 29.52, 29.44, 29.40, 29.35, 29.33, 29.31, 29.19, 29.17, 29.15, 29.12, 28.46, 28.02, 27.81, 27.23, 27.21, 27.20, 27.17, 25.86, 25.72, 25.33, 24.98, 22.68, 14.11.

### Synthesis of 115-23

Following the same procedure described for compound 242-23 above, compound 115-23 was made using 6-oxohexyl oleate (0.10 g, 0.26 mmol), N¹, N¹-dimethylpropane-1,3-diamine (0.03 g, 0.26 mmol), oleic acid (0.07 g, 0.26 mmol), and (Z)-1-isocyanooctadec-9-ene (0.07 g, 0.26 mmol) in methanol (1 ml) to give compound 115-23 as a light brown oil (0.15 g, 0.15 mmol, 58%). QTOF MS (ESI): m/z calcd for C₆₆H₁₂₆N₃O₄⁺ (M+H), 1024.97483; found, 1024.9746. ¹H NMR (400 MHz, CDCl₃) δ 6.69 (t, J=5.9 Hz, 1H), 5.43-5.29 (m, 6H), 4.73 (t, J=7.7 Hz, 1H), 4.05 (q, J=6.5 Hz, 2H), 3.32 (ddt, J=26.6, 10.1, 5.0 Hz, 2H), 3.17 (p, J=6.6 Hz, 2H), 2.38 (td, J=7.4, 4.7 Hz, 2H), 2.33-2.15 (m, 11H), 2.01 (q, J=6.5 Hz, 13H), 1.84-1.54 (m, 10H), 1.54-1.11 (m, 68H), 0.88 (t, J=6.7 Hz, 9H). ¹³C NMR (101 MHz, CDCl₃) δ 174.98, 173.91, 171.47, 130.00, 129.97, 129.95, 129.75, 129.69, 64.11, 56.74, 45.42, 39.25, 34.33, 33.54, 32.61, 32.59, 31.90, 31.87, 29.76, 29.70, 29.66, 29.64, 29.60, 29.52, 29.50, 29.49, 29.46, 29.41, 29.36, 29.32, 29.26, 29.21, 29.18, 29.15, 29.12, 28.53, 28.00, 27.82, 27.21, 27.19, 27.17, 26.90, 26.00, 25.83, 25.59, 24.97, 22.68, 22.66, 14.11, 14.07.

### Synthesis of 119-23

Following the same procedure described for compound 242-23 above, compound 119-23 was made using 6-oxohexyl oleate (0.10 g, 0.26 mmol), N¹, N¹-dimethylpropane-1,3-diamine (0.03 g, 0.26 mmol), oleic acid (0.07 g, 0.26 mmol), and 1-adamantyl isocyanide (0.04 g, 0.26 mmol) in methanol (1 ml) to give compound 119-23 as a light brown oil (0.12 g, 0.13 mmol, 50%). QTOF MS (ESI): m/z calcd for C₅₈H₁₀₆N₃O₄⁺ (M+H), 908.81833; found, 908.8183. ¹H NMR (400 MHz, CDCl₃) δ 6.36 (s, 1H), 5.42-5.26 (m, 4H), 4.72 (t, J=7.6 Hz, 1H), 4.05 (q, J=7.0, 6.6 Hz, 2H), 3.31 (ddt, J=28.2, 9.6, 4.8 Hz, 2H), 2.47-2.33 (m, 2H), 2.34-2.15 (m, 10H), 2.11-1.88 (m, 18H), 1.87-1.75 (m, 1H), 1.74-1.53 (m, 14H), 1.31 (pd, J=11.6, 10.6, 5.9 Hz, 44H), 0.89 (t, J=6.7 Hz, 6H). ¹³C NMR (101 MHz, CDCl₃) δ 174.81, 173.91, 170.46, 130.00, 129.97, 129.76, 129.69, 64.15, 56.85, 51.57, 45.39, 41.48, 36.35, 34.34, 33.45, 31.90, 29.76, 29.70, 29.51, 29.45, 29.41, 29.37, 29.31, 29.23, 29.18, 29.15, 29.11, 28.53, 27.91, 27.86, 27.21, 27.19, 27.17, 25.96, 25.86, 25.70, 24.97, 22.67, 14.11.

### Synthesis of 1110-23

Following the same procedure described for compound 242-23 above, compound 1110-23 was made using 6-oxohexyl oleate (0.10 g, 0.26 mmol), N¹, N¹-dimethylpropane-1,3-diamine (0.03 g, 0.26 mmol), oleic acid (0.07 g, 0.26 mmol), and 1-(3-isocyanopropyl)-1H-pyrazole (0.04 g, 0.26 mmol) in methanol (1 ml) to give compound 1110-23 as a light brown oil (0.12 g, 0.13 mmol, 50%). QTOF MS (ESI): v/z calcd for C₅₄H₁₀₀N₅O₄⁺ (M+H), 882.77753; found, 882.7771. ¹H NMR (400 MHz, CDCl₃) δ 7.48 (d, J=1.9 Hz, 1H), 7.41 (d, J=2.3 Hz, 1H), 6.95 (t, J=6.3 Hz, 1H), 6.22 (t, J=2.1 Hz, 1H), 5.34 (td, J=7.1, 6.0, 4.1 Hz, 4H), 4.66 (t, J=7.7 Hz, 1H), 4.21-4.08 (m, 2H), 4.04 (q, J=7.1 Hz, 2H), 3.42-3.23 (m, 2H), 3.15 (qd, J=6.7, 1.6 Hz, 2H), 2.38 (td, J=7.4, 2.9 Hz, 2H), 2.32-2.10 (m, 11H), 2.10-1.90 (m, 11H), 1.81-1.53 (m, 10H), 1.48-1.12 (m, J=7.4 Hz, 44H), 0.95-0.81 (m, 6H). ¹³C NMR (101 MHz, CDCl₃) δ 174.96, 173.89, 171.78, 139.40, 129.99, 129.96, 129.74, 129.69, 129.29, 105.41, 64.06, 56.74, 49.27, 45.43, 36.40, 34.31, 33.58, 31.88, 30.20, 29.75, 29.73, 29.68, 29.50, 29.44, 29.36, 29.30, 29.17, 29.13, 29.10, 28.51, 28.08, 27.87, 27.20, 27.17, 27.15, 26.04, 25.80, 25.52, 24.96, 22.66, 22.64, 14.10, 14.09.

### Synthesis of 1142-23

Following the same procedure described for compound 242-23 above, compound 1142-23 was made using 6-oxohexyl (9Z,12Z,15Z)-octadeca-9,12,15-trienoate (0.10 g, 0.27 mmol), N¹, N¹-dimethylpropane-1,3-diamine (0.03 g, 0.27 mmol), 6-(heptadecan-9-yloxy)-6-oxohexanoic acid (0.10 g, 0.27 mmol), and tert-butyl isocyanide (0.02 g, 0.27 mmol) in methanol (1 ml) to give compound 1142-23 0.71 g, 0.27 mmol, 30%) as a light brown oil (QTOF MS (ESI): m/z calcd for C₅₇H₁₀₆N₃O₆⁺ (M+H), 928.80816; found, 928.8082. ¹H NMR (400 MHz, CDCl₃) δ 6.52 (s, 1H), 5.47-5.32 (m, 4H), 4.87 (p, J=6.3 Hz, 1H), 4.70 (t, J=7.7 Hz, 1H), 4.04 (t, J=6.6 Hz, 2H), 3.46-3.19 (m, 2H), 2.82 (t, J=6.1 Hz, 3H), 2.48-2.21 (m, 15H), 2.08 (dd, J=14.3, 7.2 Hz, 4H), 1.95 (ddd, J=10.3, 4.9, 2.4 Hz, 1H), 1.81 (dt, J=11.1, 6.5 Hz, 1H), 1.76-1.57 (m, 12H), 1.51 (q, J=6.3 Hz, 5H), 1.30 (t, J=10.6 Hz, 46H), 0.99 (t, J=7.5 Hz, 3H), 0.89 (t, J=6.7 Hz, 5H). ¹³C NMR (101 MHz, CDCl₃) δ 174.28, 173.93, 173.16, 170.60, 131.95, 130.29, 128.28, 128.26, 127.71, 127.12, 74.35, 64.15, 56.64, 50.89, 45.23, 34.37, 34.33, 34.10, 33.10, 31.86, 29.59, 29.54, 29.50, 29.25, 29.18, 29.15, 29.12, 28.62, 28.57, 28.52, 27.90, 27.64, 27.21, 25.97, 25.85, 25.61, 25.53, 25.33, 25.04, 24.97, 24.81, 22.66, 20.55, 14.28, 14.11.

### Synthesis of 122-24

Following the same procedure described for compound 242-23 above, compound 111-6 was made using 6-oxohexyl oleate (0.1 g, 0.26 mmol), 3,3′-(piperazine-1,4-diyl) bis(propan-1-amine) (0.1 g, 5.3 mmol), linoleic acid (0.07 g, 0.26 mmol), and tert-butyl isocyanide (0.02 g, 0.26 mmol) in methanol (1 ml) to give a brown oil (0.05 g, 0.05 mmol, 17%). QTOF MS (ESI): m/z calcd for C₅₇H₁₀₈N₅O₄⁺ (M+H), 926.84013; found, 926.8404. ¹H NMR (400 MHz, CDCl₃) δ 6.53 (d, J=8.7 Hz, 1H), 5.37 (th, J=10.9, 5.7 Hz, 5H), 4.73 (d, J=17.8 Hz, 1H), 4.07 (dt, J=13.3, 6.7 Hz, 3H), 3.44-3.19 (m, 2H), 2.78 (td, J=6.7, 4.5 Hz, 3H), 2.60-2.36 (m, 9H), 2.30 (td, J=7.4, 4.1 Hz, 5H), 2.13-1.98 (m, 8H), 1.98-1.84 (m, 4H), 1.81 (q, J=6.3, 5.4 Hz, 1H), 1.64 (tdd, J=13.7, 9.5, 5.5 Hz, 11H), 1.32 (h, J=5.7 Hz, 49H), 0.90 (td, J=6.8, 4.0 Hz, 6H). ¹³C NMR (101 MHz, CDCl₃) δ 174.92, 173.95, 170.71, 130.27, 130.00, 129.77, 128.10, 127.88, 74.81, 64.15, 56.54, 55.38, 53.31, 53.25, 50.85, 40.83, 34.35, 33.54, 31.91, 31.53, 30.27, 29.77, 29.72, 29.67, 29.53, 29.44, 29.35, 29.33, 29.23, 29.20, 29.17, 29.13, 28.65, 28.55, 27.86, 27.23, 27.21, 27.18, 25.97, 25.86, 25.74, 25.64, 24.99, 22.69, 22.58, 14.13, 14.09.

### Synthesis of 142-23

Following the same procedure described for compound 242-23 above, compound 142-23 was made using 6-oxohexyl oleate (0.10 g, 0.26 mmol), N¹, N¹-dimethylpropane-1,3-diamine (0.027 g, 0.26 mmol), 6-(heptadecan-9-yloxy)-6-oxohexanoic acid (0.10 g, 0.26 mmol) and tert-butyl isocyanide (0.02 g, 0.26 mmol) to give compound 142-23 as a light brown oil (0.086 g, 0.09 mmol, 35%). QTOF MS (ESI): m/z calcd for C₅₇H₁₁₀N₃O₆⁺ (M+H), 932.83946; found, 932.8414. ¹H NMR (400 MHz, CDCl₃) δ 6.51 (s, 1H), 5.35 (td, J=6.8, 5.8, 4.0 Hz, 2H), 4.87 (p, J=6.1 Hz, 1H), 4.70 (t, J=7.7 Hz, 1H), 4.06 (dt, J=13.2, 6.7 Hz, 2H), 3.36 (dt, J=10.6, 5.4 Hz, 1H), 3.26 (ddd, J=17.8, 8.8, 3.8 Hz, 1H), 2.42 (dd, J=7.3, 3.2 Hz, 2H), 2.37-2.18 (m, 12H), 2.02 (q, J=6.6 Hz, 5H), 1.89-1.75 (m, 1H), 1.75-1.56 (m, 10H), 1.51 (d, J=6.3 Hz, 4H), 1.43-1.18 (m, 56H), 0.89 (t, J=6.7 Hz, 8H). ¹³C NMR (101 MHz, CDCl₃) δ 174.29, 173.93, 173.15, 170.61, 129.97, 129.76, 74.33, 64.13, 56.74, 50.86, 45.38, 34.37, 34.34, 34.10, 33.09, 31.90, 31.88, 31.85, 29.76, 29.70, 29.68, 29.65, 29.61, 29.53, 29.52, 29.49, 29.36, 29.31, 29.28, 29.26, 29.24, 29.21, 29.18, 29.15, 29.12, 28.62, 28.56, 28.52, 27.87, 27.80, 27.21, 27.19, 27.17, 25.97, 25.85, 25.32, 25.05, 24.97, 24.81, 22.68, 22.66, 14.10, 14.07.

### Synthesis of 241-1

Following the same procedure described for compound 242-23 above, compound 241-1 was made using 6-oxohexyl (9Z,12Z)-octadeca-9,12-dienoate (0.05 g, 0.13 mmol), 2-(pyrrolidin-1-yl)ethan-1-amine (0.015 g, 0.13 mmol), 6-(heptadecan-9-yloxy)-6-oxohexanoic acid (0.05 g, 0.13 mmol) and tert-butyl 2-isocyanoacetate (0.019 g, 0.13 mmol) in methanol (1 ml) to give 241-1 as a light brown oil (0.017 g, 0.017 mmol, 13%). QTOF MS (ESI): m/z calcd for C₆₀H₁₁₀N₃O₈⁺ (M+H), 1000.82929; found, 1000.8290. ¹H NMR (400 MHz, CDCl₃) δ 5.45-5.33 (m, 5H), 4.86 (p, J=6.2 Hz, 1H), 4.06 (td, J=6.6, 3.5 Hz, 3H), 3.89-3.83 (m, 1H), 2.79 (t, J=6.5 Hz, 3H), 2.67-2.52 (m, 4H), 2.45 (t, J=6.9 Hz, 1H), 2.38-2.23 (m, 6H), 2.06 (dd, J=8.0, 5.7 Hz, 6H), 1.87-1.57 (m, 16H), 1.57-1.44 (m, 13H), 1.44-1.20 (m, 44H), 0.90 (td, J=6.8, 4.9 Hz, 9H). ¹³C NMR (101 MHz, CDCl₃) δ 173.93, 173.26, 173.21, 171.58, 168.78, 130.22, 130.08, 128.03, 127.93, 81.53, 74.33, 74.18, 64.13, 64.02, 55.25, 54.24, 53.43, 41.89, 34.34, 34.11, 33.78, 33.18, 31.87, 31.53, 29.62, 29.56, 29.50, 29.35, 29.27, 29.20, 29.16, 29.14, 28.52, 28.04, 28.01, 27.21, 25.85, 25.63, 25.33, 24.98, 24.82, 24.75, 24.67, 23.53, 23.49, 22.67, 22.58, 14.11, 14.08.

### Synthesis of 244-23

Following the same procedure described for compound 242-23 above, compound 244-23 was made using 6-oxohexyl (9Z,12Z)-octadeca-9,12-dienoate (0.05 g, 0.13 mmol), N¹, N¹-dimethylpropane-1,3-diamine (0.013 g, 0.13 mmol), 6-(heptadecan-9-yloxy)-6-oxohexanoic acid (0.05 g, 0.13 mmol) and 1-isocyanobutane (0.011 g, 0.13 mmol) in methanol (1 ml) to give 241-1 as a light brown oil (0.050 g, 0.05 mmol, 38%). QTOF MS (ESI): m/z calcd for C₅₇H₁₀₈N₃O₆⁺ (M+H), 930.82381; found, 930.8244. ¹H NMR (400 MHz, CDCl₃) δ 6.67 (s, 1H), 5.49-5.27 (m, 4H), 4.88 (p, J=6.2 Hz, 1H), 4.73 (t, J=7.7 Hz, 1H), 4.06 (q, J=6.6 Hz, 2H), 3.45-3.13 (m, 4H), 2.79 (t, J=6.4 Hz, 2H), 2.44 (dq, J=6.8, 3.6 Hz, 2H), 2.32 (dt, J=18.5, 7.4 Hz, 4H), 2.27-2.16 (m, 7H), 2.07 (q, J=6.8 Hz, 6H), 1.78-1.58 (m, 15H), 1.52 (t, J=6.2 Hz, 4H), 1.48-1.20 (m, 43H), 1.04-0.80 (m, 11H). ¹³C NMR (101 MHz, CDCl₃) δ 174.39, 173.17, 171.41, 130.23, 130.08, 128.04, 127.93, 74.37, 64.14, 56.71, 45.48, 38.97, 34.35, 34.11, 33.17, 31.87, 31.54, 31.51, 29.62, 29.55, 29.51, 29.36, 29.26, 29.20, 29.17, 29.15, 28.54, 28.06, 27.84, 27.21, 26.06, 25.86, 25.64, 25.34, 24.99, 24.82, 22.67, 22.58, 20.03, 14.11, 14.08, 13.71.

### Synthesis of 247-23

Following the same procedure described for compound 242-23 above, compound 247-23 was made using 6-oxohexyl (9Z,12Z)-octadeca-9,12-dienoate (0.05 g, 0.13 mmol), N¹, N¹-dimethylpropane-1,3-diamine (0.013 g, 0.13 mmol), 6-(heptadecan-9-yloxy)-6-oxohexanoic acid (0.05 g, 0.13 mmol) and N-butyl-N-(3-isocyanopropyl)butan-1-amine (0.03 g, 0.13 mmol) in methanol (1 ml) to give 247-23 (0.023 g, 0.05 mmol, 17%) as a light brown oil. QTOF MS (ESI): m/z calcd for C₆₄H₁₂₃N₄O₆⁺ (M+H), 1043.94426; found, 1043.9440. ¹H NMR (400 MHz, CDCl₃) δ 7.07 (t, J=5.6 Hz, 1H), 5.47-5.32 (m, 4H), 4.88 (p, J=6.2 Hz, 1H), 4.76 (t, J=7.6 Hz, 1H), 4.06 (dt, J=10.8, 6.7 Hz, 3H), 3.41-3.13 (m, 4H), 2.79 (t, J=6.4 Hz, 2H), 2.50-2.26 (m, 12H), 2.23 (d, J=13.6 Hz, 9H), 2.06 (q, J=6.8 Hz, 5H), 1.65 (ddtd, J=24.1, 10.0, 7.2, 6.7, 3.8 Hz, 14H), 1.52 (d, J=6.2 Hz, 4H), 1.47-1.18 (m, 49H), 0.91 (dt, J=11.7, 7.2 Hz, 14H). ¹³C NMR (101 MHz, CDCl₃) δ 173.98, 173.92, 173.18, 171.18, 130.22, 130.07, 128.03, 127.92, 74.34, 64.15, 56.82, 53.86, 52.15, 45.50, 45.45, 38.43, 34.38, 34.34, 34.11, 33.13, 31.86, 31.53, 29.62, 29.55, 29.51, 29.40, 29.35, 29.26, 29.20, 29.16, 29.14, 29.07, 28.56, 28.35, 28.01, 27.21, 26.71, 26.06, 25.88, 25.63, 25.33, 25.02, 24.98, 24.84, 22.67, 22.65, 22.58, 20.75, 20.65, 14.11, 14.08.

### Synthesis of 249-23

Following the same procedure described for compound 242-23 above, compound 249-23 was made using 6-oxohexyl (9Z,12Z)-octadeca-9,12-dienoate (0.05 g, 0.13 mmol), N¹, N¹-dimethylpropane-1,3-diamine (0.013 g, 0.13 mmol), 6-(heptadecan-9-yloxy)-6-oxohexanoic acid (0.05 g, 0.13 mmol) and 1-adamantyl isocyanide (0.02 g, 0.13 mmol) in methanol (1 ml) to give 249-23 (0.023 g, 0.05 mmol, 17%) as a light brown oil. QTOF MS (ESI): m/z calcd for C₆₃H₁₁₄N₃O₆⁺ (M+H), 1008.87076; found, 1008.8705. ¹H NMR (400 MHz, CDCl₃) δ 6.34 (s, 1H), 5.37 (tdd, J=11.0, 7.9, 3.6 Hz, 4H), 4.88 (p, J=6.2 Hz, 1H), 4.72 (t, J=7.6 Hz, 1H), 4.06 (q, J=7.1, 6.7 Hz, 2H), 3.43-3.18 (m, 2H), 2.79 (t, J=6.5 Hz, 2H), 2.48-2.39 (m, 2H), 2.39-2.25 (m, 6H), 2.22 (d, J=4.6 Hz, 6H), 2.15-2.01 (m, 8H), 1.95 (d, J=3.0 Hz, 6H), 1.82 (q, J=9.4, 7.2 Hz, 4H), 1.75-1.57 (m, 15H), 1.52 (q, J=6.1 Hz, 4H), 1.46-1.11 (m, 41H), 0.90 (td, J=6.8, 4.6 Hz, 8H). ¹³C NMR (101 MHz, CDCl₃) δ 174.23, 173.94, 173.18, 170.37, 130.22, 130.08, 128.03, 127.93, 74.35, 64.18, 56.83, 53.43, 51.61, 45.47, 41.48, 36.34, 34.38, 34.35, 34.10, 33.10, 31.86, 31.53, 29.70, 29.62, 29.55, 29.50, 29.43, 29.38, 29.35, 29.26, 29.20, 29.16, 29.14, 28.54, 27.94, 27.21, 26.00, 25.88, 25.63, 25.33, 25.08, 24.98, 24.84, 22.67, 22.58, 14.11, 14.08.

### Synthesis of 2410-23

Following the same procedure described for compound 242-23 above, compound 2410-23 was made using 6-oxohexyl (9Z,12Z)-octadeca-9,12-dienoate (0.1 g, 0.26 mmol), N¹, N¹-dimethylpropane-1,3-diamine (0.03 g, 0.26 mmol), 6-(heptadecan-9-yloxy)-6-oxohexanoic acid (0.10 g, 0.26 mmol) and 1-(3-isocyanopropyl)-1H-pyrazole (0.04 g, 0.13 mmol) in methanol (1 ml) to give 2410-23 (0.10 g, 0.10 mmol, 38%) as a light brown oil. QTOF MS (ESI): m/z calcd for C₅₉H₁₀₈N₅O₆⁺ (M+H), 982.82996; found, 982.8302. ¹H NMR (400 MHz, CDCl₃) δ 7.48 (d, J=1.9 Hz, 1H), 7.41 (d, J=2.2 Hz, 1H), 6.92 (t, J=6.1 Hz, 1H), 6.22 (t, J=2.1 Hz, 1H), 5.45-5.24 (m, 4H), 4.85 (p, J=6.3 Hz, 1H), 4.66 (t, J=7.6 Hz, 1H), 4.18-4.09 (m, 2H), 4.03 (t, J=6.7 Hz, 2H), 3.32 (dt, J=9.6, 6.0 Hz, 2H), 3.16 (qd, J=6.7, 3.2 Hz, 2H), 2.76 (t, J=6.4 Hz, 2H), 2.41 (tt, J=8.2, 4.4 Hz, 2H), 2.36-2.15 (m, 13H), 2.02 (dt, J=9.0, 6.4 Hz, 8H), 1.78-1.55 (m, 12H), 1.50 (q, J=6.3 Hz, 4H), 1.43-1.14 (m, 42H), 0.88 (td, J=6.8, 4.9 Hz, 9H). ¹³C NMR (101 MHz, CDCl₃) δ 174.33, 173.88, 173.16, 171.67, 139.39, 130.18, 130.03, 129.28, 128.01, 127.90, 105.43, 74.33, 64.07, 56.69, 49.28, 45.43, 36.43, 34.31, 34.08, 33.17, 31.84, 31.82, 31.50, 30.20, 29.67, 29.59, 29.52, 29.48, 29.35, 29.33, 29.31, 29.25, 29.23, 29.17, 29.13, 29.11, 28.51, 28.12, 27.83, 27.18, 26.08, 25.81, 25.61, 25.31, 24.95, 24.92, 24.76, 22.64, 22.55, 14.09, 14.06, 14.04.

### Synthesis of 613-23

Following the same procedure described for compound 242-23 above, compound 613-23 was made using 6-oxohexyl undecanoate (0.05 g, 0.18 mmol), N¹, N¹-dimethylpropane-1,3-diamine (0.018 g, 0.18 mmol), oleic acid (0.05 g, 0.18 mmol), and isocyanocyclohexane (0.019 g, 0.18 mmol) in methanol (1 ml) to give compound 613-23 a light brown oil (0.035 g, 0.05 mmol, 28%). QTOF MS (ESI): m/z calcd for C₄₇H₉₀N₃O₄⁺ (M+H), 760.69313; found, 760.6928. ¹H NMR (400 MHz, CDCl₃) δ 6.61 (d, J=8.6 Hz, 1H), 5.44-5.27 (m, 2H), 4.73 (d, J=7.5 Hz, 1H), 4.05 (t, J=6.6 Hz, 3H), 3.78-3.63 (m, 1H), 3.33 (ddt, J=24.9, 9.9, 5.0 Hz, 2H), 2.41 (ddt, J=10.5, 7.2, 4.1 Hz, 2H), 2.30 (td, J=7.6, 3.1 Hz, 3H), 2.23 (d, J=10.5 Hz, 7H), 2.03 (dt, J=13.5, 6.3 Hz, 4H), 1.91-1.74 (m, 4H), 1.65 (dq, J=14.9, 7.1, 5.9 Hz, 12H), 1.45-1.21 (m, 39H), 1.21-1.03 (m, 3H), 0.90 (t, J=6.7 Hz, 6H). ¹³C NMR (101 MHz, CDCl₃) δ 175.01, 173.98, 170.59, 130.03, 129.71, 64.14, 56.80, 47.75, 45.48, 34.37, 33.52, 32.83, 32.74, 31.90, 29.77, 29.57, 29.53, 29.48, 29.46, 29.41, 29.34, 29.31, 29.28, 29.23, 29.19, 28.57, 28.55, 27.93, 27.89, 27.23, 27.20, 26.00, 25.85, 25.66, 25.53, 25.01, 24.59, 22.68, 14.12.

### Synthesis of 622-23

Following the same procedure described for compound 242-23 above, compound 622-23 was made using 6-oxohexyl undecanoate (0.05 g, 0.18 mmol), N¹, N¹-dimethylpropane-1,3-diamine (0.018 g, 0.18 mmol), linoleic acid (0.05 g, 0.18 mmol), and tert-butyl isocyanide (0.015 g, 0.18 mmol) in methanol (1 ml) to give a light brown oil (0.050 g, 0.05 mmol, 38%). QTOF MS (ESI): m/z calcd for C₄₅H₈₆N₃O₄⁺ (M+H), 732.66183; found, 732.6619. ¹H NMR (400 MHz, CDCl₃) δ 6.52 (d, J=7.1 Hz, 1H), 5.47-5.26 (m, 2H), 4.77-4.61 (m, 1H), 4.06 (dt, J=13.2, 6.7 Hz, 3H), 3.32 (ddt, J=29.9, 9.6, 4.7 Hz, 2H), 2.78 (t, J=6.4 Hz, 1H), 2.48-2.34 (m, 2H), 2.33-2.18 (m, 10H), 2.11-2.02 (m, 3H), 1.87-1.75 (m, 1H), 1.75-1.55 (m, 9H), 1.49-1.18 (m, 42H), 0.90 (td, J=6.8, 4.6 Hz, 6H). ¹³C NMR (101 MHz, CDCl₃) δ 174.89, 173.98, 170.70, 130.24, 130.01, 128.07, 127.89, 64.13, 64.12, 64.06, 56.78, 50.85, 45.34, 34.36, 33.49, 31.89, 31.52, 29.65, 29.56, 29.47, 29.45, 29.40, 29.34, 29.30, 29.27, 29.21, 29.18, 28.63, 28.57, 28.53, 27.84, 27.20, 25.95, 25.85, 25.69, 25.62, 24.99, 22.68, 22.57, 14.11, 14.08.

The following lipids were synthesized as described above and only characterized using mass spectrometry:

111-1

QTOF MS (ESI): m/z calcd for C₅₅H₁₀₂N₃O₆⁺ (M+H), 900.77686; found, 900.7768.

111-2

QTOF MS (ESI): m/z calcd for C₅₆H₁₀₄N₄O₆⁺ (M+H), 913.78469; found, 914.7923.

111-9

QTOF MS (ESI): m/z calcd for C₅₃H₁₀₀N₃O₆⁺ (M+H), 874.76121; found, 874.7611.

111-15

QTOF MS (ESI): m/z calcd for C₅₇H₁₀₇N₃O₆⁺ (M+H), 928.80816; found, 928.8078.

111-16

QTOF MS (ESI): m/z calcd for C₅₈H₁₀₈N₃O₆⁺ (M+H), 942.82381; found, 942.8233.

111-18

QTOF MS (ESI): m/z calcd for C₅₇H₁₀₇N₄O₆⁺ (M+H), 943.81906; found, 943.8187.

111-25

QTOF MS (ESI): m/z calcd for C₅₄H₁₀₃N₄O₆⁺ (M+H), 903.78776; found, 903.7873.

115-9

QTOF M-S(ESI): m/z calcd for C₆₅H₁₂₄N₃O₄⁺ (M+H), 1010.95918; found, 1010.9591.

117-9

QTOF MS (ESI): m/z calcd for C₅₈H₁₁₃N₄O₄⁺ (M+H), 929.87618; found, 929.87618.

122-25

QTOF MS (ESI): m/z calcd for C₅₂H₉₉N₄O₄⁺ (M+H), 843.76663; found, 843.7668.

225-25

QTOF MS (ESI): m/z calcd for C₆₆H₁₁₂N₄O₄⁺ (M+H), 1035.95443; found, 1035.9539.

241-2

QTOF MS (ESI): m/z calcd for C₆₁H₁₁₂N₃O₈⁺ (M+H), 1014.84494; found, 1014.8446.

241-9

QTOF MS (ESI): m/z calcd for C₅₈H₁₀₈N₃O₈⁺ (M+H), 974.81364; found, 974.8134.

241-18

QTOF MS (ESI): m/z calcd for C₆₂H₁₁₅N₄O₈⁺ (M+H), 1043.87149; found, 1043.8712.

241-25

QTOF MS (ESI): m/z calcd for C₅₉H₁₁₁N₄O₈⁺ (M+H), 1003.84019; found, 10030.8402.

245-9

QTOF MS (ESI): m/z calcd for C₇₀H₁₃₂N₃O₆⁺ (M+H), 1111.01161; found, 1111.0114.

245-23

QTOF MS (ESI): m/z calcd for C₇₁H₁₃₄N₃O₆⁺ (M+H), 1125.02726; found, 1125.0269.

246-23

QTOF MS (ESI): m/z calcd for C₅₈H₁₁₁N₄O₆⁺ (M+H), 959.85036; found, 959.8510.

247-9

QTOF MS (ESI): m/z calcd for C₆₃H₁₂₁N₄O₆⁺ (M+H), 1029.92861; found, 1029.9283.

248-23

QTOF MS (ESI): m/z calcd for C₆₀H₁₁₅N₄O₆⁺ (M+H), 987.88166; found, 987.8821.

249-9

QTOF MS (ESI): m/z calcd for C₆₂H₁₁₂N₃O₆⁺ (M+H), 994.85511; found, 994.8547.

621-9

QTOF MS (ESI): m/z calcd for C₄₆H₈₆N₃O₆⁺ (M+H), 776.65166; found, 776.6515.

621-23

QTOF MS (ESI): m/z calcd for C₄₇H₈₈N₃O₆⁺ (M+H), 790.66731; found, 790.6670.

### 119-23-C₁₁

QTOF MS (ESI): m/z calcd for C₄₄H₈₂N₃O₄⁺ (M+H), 716.63053; found, 716.6301.

119-23-C_{12 unsaturated}

QTOF MS (ESI): m/z calcd for C₄₆H₈₂N₃O₄⁺ (M+H), 740.63053; found, 740.6307.

### 119-23-C₁₃

QTOF MS (ESI): m/z calcd for C₄₈H₉₀N₃O₄⁺ (M+H), 772.69313; found, 772.6926.

### 119-23-C₁₆

QTOF MS (ESI): m/z calcd for C₅₄H₁₀₂N₃O₄⁺ (M+H), 856.78703; found, 856.7865.

### 119-23-C₁₈

QTOF MS (ESI): m/z calcd for C₅₈H₁₁₀N₃O₄⁺ (M+H), 912.84963; found, 912.8492.

### 119-23-Linoleic

QTOF MS (ESI): m/z calcd for C₅₈H₁₀₂N₃O₄⁺ (M+H), 904.78703; found, 904.7864.

### 119-23-Linolenic

QTOF MS (ESI): m/z calcd for C₅₈H₉₈N₃O₄⁺ (M+H), 900.75573; found, 900.7549.

### REFERENCES

1. Cornebise, M.; Narayanan, E.; Xia, Y.; Acosta, E.; Ci, L.; Koch, H.; Milton, J.; Sabnis, S.; Salerno, T.; Benenato, K. E., Discovery of a Novel Amino Lipid That Improves Lipid Nanoparticle Performance through Specific Interactions with mRNA. Advanced Functional Materials 2022, 32 (8), 2106727.
2. Elders, N.; Schmitz, R. F.; de Kanter, F. J. J.; Ruijter, E.; Groen, M. B.; Orru, R. V. A., A Resource-Efficient and Highly Flexible Procedure for a Three-Component Synthesis of 2-Imidazolines. The Journal of Organic Chemistry 2007, 72 (16), 6135-6142.
3. Miao, L.; Li, L.; Huang, Y.; Delcassian, D.; Chahal, J.; Han, J.; Shi, Y.; Sadtler, K.; Gao, W.; Lin, J.; Doloff, J. C.; Langer, R.; Anderson, D. G., Delivery of mRNA vaccines with heterocyclic lipids increases anti-tumor efficacy by STING-mediated immune cell activation. Nat. Biotechnol. 2019, 37 (10), 1174-1185.
4. Akinc, A.; Zumbuehl, A.; Goldberg, M.; Leshchiner, E. S.; Busini, V.; Hossain, N.; Bacallado, S. A.; Nguyen, D. N.; Fuller, J.; Alvarez, R.; Borodovsky, A.; Borland, T.; Constien, R.; de Fougerolles, A.; Dorkin, J. R.; Narayanannair Jayaprakash, K.; Jayaraman, M.; John, M.; Koteliansky, V.; Manoharan, M.; Nechev, L.; Qin, J.; Racie, T.; Raitcheva, D.; Rajeev, K. G.; Sah, D. W. Y.; Soutschek, J.; Toudjarska, I.; Vornlocher, H.-P.; Zimmermann, T. S.; Langer, R.; Anderson, D. G., A combinatorial library of lipid-like materials for delivery of RNAi therapeutics. Nat. Biotechnol. 2008, 26 (5), 561-569.
5. Whitehead, K. A.; Dorkin, J. R.; Vegas, A. J.; Chang, P. H.; Veiseh, O.; Matthews, J.; Fenton, O. S.; Zhang, Y.; Olejnik, K. T.; Yesilyurt, V.; Chen, D.; Barros, S.; Klebanov, B.; Novobrantseva, T.; Langer, R.; Anderson, D. G., Degradable lipid nanoparticles with predictable in vivo siRNA delivery activity. Nature Communications 2014, 5 (1), 4277.
6. Qiu, M.; Tang, Y.; Chen, J.; Muriph, R.; Ye, Z.; Huang, C.; Evans, J.; Henske, E. P.; Xu, Q., Lung-selective mRNA delivery of synthetic lipid nanoparticles for the treatment of pulmonary lymphangioleiomyomatosis. Proceedings of the National Academy of Sciences 2022, 119 (8), e2116271119.
7. Dong, Y.; Love, K. T.; Dorkin, J. R.; Sirirungruang, S.; Zhang, Y.; Chen, D.; Bogorad, R. L.; Yin, H.; Chen, Y.; Vegas, A. J.; Alabi, C. A.; Sahay, G.; Olejnik, K. T.; Wang, W.; Schroeder, A.; Lytton-Jean, A. K. R.; Siegwart, D. J.; Akinc, A.; Barnes, C.; Barros, S. A.; Carioto, M.; Fitzgerald, K.; Hettinger, J.; Kumar, V.; Novobrantseva, T. I.; Qin, J.; Querbes, W.; Koteliansky, V.; Langer, R.; Anderson, D. G., Lipopeptide nanoparticles for potent and selective siRNA delivery in rodents and nonhuman primates. Proceedings of the National Academy of Sciences 2014, 111 (11), 3955-3960.
8. Maier, M. A.; Jayaraman, M.; Matsuda, S.; Liu, J.; Barros, S.; Querbes, W.; Tam, Y. K.; Ansell, S. M.; Kumar, V.; Qin, J.; Zhang, X.; Wang, Q.; Panesar, S.; Hutabarat, R.; Carioto, M.; Hettinger, J.; Kandasamy, P.; Butler, D.; Rajeev, K. G.; Pang, B.; Charisse, K.; Fitzgerald, K.; Mui, B. L.; Du, X.; Cullis, P.; Madden, T. D.; Hope, M. J.; Manoharan, M.; Akinc, A., Biodegradable Lipids Enabling Rapidly Eliminated Lipid Nanoparticles for Systemic Delivery of RNAi Therapeutics. Mol. Ther. 2013, 21 (8), 1570-1578.
9. Kaczmarek, J. C.; Kauffman, K. J.; Fenton, O. S.; Sadtler, K.; Patel, A. K.; Heartlein, M. W.; DeRosa, F.; Anderson, D. G., Optimization of a Degradable Polymer-Lipid Nanoparticle for Potent Systemic Delivery of mRNA to the Lung Endothelium and Immune Cells. Nano Lett 2018, 18 (10), 6449-6454.
10. Ekins, S.; Puhl, A. C.; Zorn, K. M.; Lane, T. R.; Russo, D. P.; Klein, J. J.; Hickey, A. J.; Clark, A. M., Exploiting machine learning for end-to-end drug discovery and development. Nature Materials 2019, 18 (5), 435-441.
11. Yap, C. W., PaDEL-descriptor: an open source software to calculate molecular descriptors and fingerprints. J Comput Chem 2011, 32 (7), 1466-74.
12. Kauffman, K. J.; Dorkin, J. R.; Yang, J. H.; Heartlein, M. W.; DeRosa, F.; Mir, F. F.; Fenton, O. S.; Anderson, D. G., Optimization of Lipid Nanoparticle Formulations for mRNA Delivery in Vivo with Fractional Factorial and Definitive Screening Designs. Nano Letters 2015, 15 (11), 7300-7306.
13. Cheng, Q.; Wei, T.; Farbiak, L.; Johnson, L. T.; Dilliard, S. A.; Siegwart, D. J., Selective organ targeting (SORT) nanoparticles for tissue-specific mRNA delivery and CRISPR-Cas gene editing. Nat Nanotechnol 2020, 15 (4), 313-320.
14. Sahu, I.; Haque, A. K. M. A.; Weidensee, B.; Weinmann, P.; Kormann, M. S. D., Recent Developments in mRNA-Based Protein Supplementation Therapy to Target Lung Diseases. Molecular therapy: the journal of the American Society of Gene Therapy 2019, 27 (4), 803-823.
15. Chakraborty, C.; Sharma, A. R.; Bhattacharya, M.; Lee, S.-S., From COVID-19 to Cancer mRNA Vaccines: Moving From Bench to Clinic in the Vaccine Landscape. Front. Immunol. 2021, 12.
16. Gan, Z.; Lokugamage, M. P.; Hatit, M. Z. C.; Loughrey, D.; Paunovska, K.; Sato, M.; Cristian, A.; Dahlman, J. E., Nanoparticles containing constrained phospholipids deliver mRNA to liver immune cells in vivo without targeting ligands. Bioengineering & Translational Medicine 2020, 5 (3), e10161.
17. Kaczmarek, J. C.; Kauffman, K. J.; Fenton, O. S.; Sadtler, K.; Patel, A. K.; Heartlein, M. W.; DeRosa, F.; Anderson, D. G., Optimization of a Degradable Polymer-Lipid Nanoparticle for Potent Systemic Delivery of mRNA to the Lung Endothelium and Immune Cells. Nano Letters 2018, 18 (10), 6449-6454.
18. Oberli, M. A.; Reichmuth, A. M.; Dorkin, J. R.; Mitchell, M. J.; Fenton, O. S.; Jaklenec, A.; Anderson, D. G.; Langer, R.; Blankschtein, D., Lipid Nanoparticle Assisted mRNA Delivery for Potent Cancer Immunotherapy. Nano Letters 2017, 17 (3), 1326-1335.
19. Jayaraman, M.; Ansell, S. M.; Mui, B. L.; Tam, Y. K.; Chen, J.; Du, X.; Butler, D.; Eltepu, L.; Matsuda, S.; Narayanannair, J. K.; et al. Maximizing the Potency of SiRNA Lipid Nanoparticles for Hepatic Gene Silencing In Vivo. Angew. Chemie Int. Ed. 2012, 51 (34), 8529-8533.
20. Liu, S.; Cheng, Q.; Wei, T.; Yu, X.; Johnson, L. T.; Farbiak, L.; Siegwart, D. J. Membrane-Destabilizing Ionizable Phospholipids for Organ-Selective MRNA Delivery and CRISPR-Cas Gene Editing. Nat. Mater. 2021, 20 (5), 701-710.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included.

Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims.

Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I): 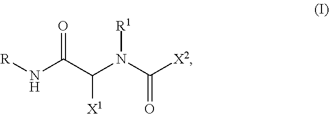
or a pharmaceutically acceptable salt, co-crystal, tautomer, stereoisomer, solvate, hydrate, polymorph, or isotopically enriched derivative thereof, wherein:
R is substituted or unsubstituted alkyl, substituted or unsubstituted heteroalkyl, or substituted or unsubstituted carbocyclyl;
R¹ is substituted or unsubstituted alkyl, substituted or unsubstituted heteroalkyl, or substituted or unsubstituted heterocyclyl; and
X¹ and X² are each independently substituted or unsubstituted C₈-C₁₀₀ aliphatic, or substituted or unsubstituted C₈-C₁₀₀ heteroaliphatic.

2. The compound of claim 1, or a pharmaceutically acceptable salt, co-crystal, tautomer, stereoisomer, solvate, hydrate, polymorph, or isotopically enriched derivative thereof, wherein X¹ is substituted or unsubstituted C₁₂-C₃₀ heteroaliphatic.

3. The compound of claim 1 or 2, or a pharmaceutically acceptable salt, co-crystal, tautomer, stereoisomer, solvate, hydrate, polymorph, or isotopically enriched derivative thereof, wherein X¹ is substituted or unsubstituted C₁₂-C₂₄ heteroaliphatic.

4. The compound of any one of claims 1-3, or a pharmaceutically acceptable salt, co-crystal, tautomer, stereoisomer, solvate, hydrate, polymorph, or isotopically enriched derivative thereof, wherein X¹ is of the formula:

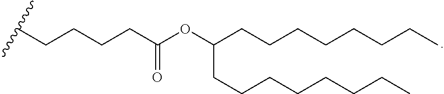 [SMILES: *CCCCC(=O)OC(CCCCCCCC)CCCCCCCC]

5. The compound of any one of claims 1-3, or a pharmaceutically acceptable salt, co-crystal, tautomer, stereoisomer, solvate, hydrate, polymorph, or isotopically enriched derivative thereof, wherein X¹ is of the formula: 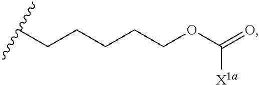
wherein X^{1a} is substituted or unsubstituted C₈-C₁₀₀ aliphatic, or substituted or unsubstituted C₈-C₁₀₀ heteroaliphatic.

6. The compound of claim 5, or a pharmaceutically acceptable salt, co-crystal, tautomer, stereoisomer, solvate, hydrate, polymorph, or isotopically enriched derivative thereof, of Formula (II): 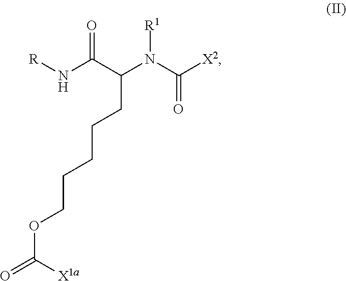
wherein:
R is substituted or unsubstituted alkyl, substituted or unsubstituted heteroalkyl, or substituted or unsubstituted carbocyclyl;
R¹ is substituted or unsubstituted alkyl, substituted or unsubstituted heteroalkyl, or substituted or unsubstituted heterocyclyl;
X² is substituted or unsubstituted C₈-C₁₀₀ aliphatic, or substituted or unsubstituted C₈-C₁₀₀ heteroaliphatic; and
X^{1a} is substituted or unsubstituted C₈-C₁₀₀ aliphatic, or substituted or unsubstituted C₈-C₁₀₀ heteroaliphatic.

7. The compound of any one of claims 4-6, or a pharmaceutically acceptable salt, co-crystal, tautomer, stereoisomer, solvate, hydrate, polymorph, or isotopically enriched derivative thereof, wherein X^{1a} is substituted or unsubstituted C₈-C₁₀₀ aliphatic.

8. The compound of any one of claims 4-7, or a pharmaceutically acceptable salt, co-crystal, tautomer, stereoisomer, solvate, hydrate, polymorph, or isotopically enriched derivative thereof, wherein X^{1a} is substituted or unsubstituted C₈-C₁₈ aliphatic.

9. The compound of any one of claims 4-7, or a pharmaceutically acceptable salt, co-crystal, tautomer, stereoisomer, solvate, hydrate, polymorph, or isotopically enriched derivative thereof, wherein X^{1a} is unsubstituted C₈-C₁₈ aliphatic.

10. The compound of any one of claims 1-9, or a pharmaceutically acceptable salt, co-crystal, tautomer, stereoisomer, solvate, hydrate, polymorph, or isotopically enriched derivative thereof, wherein X¹ is of the formula:

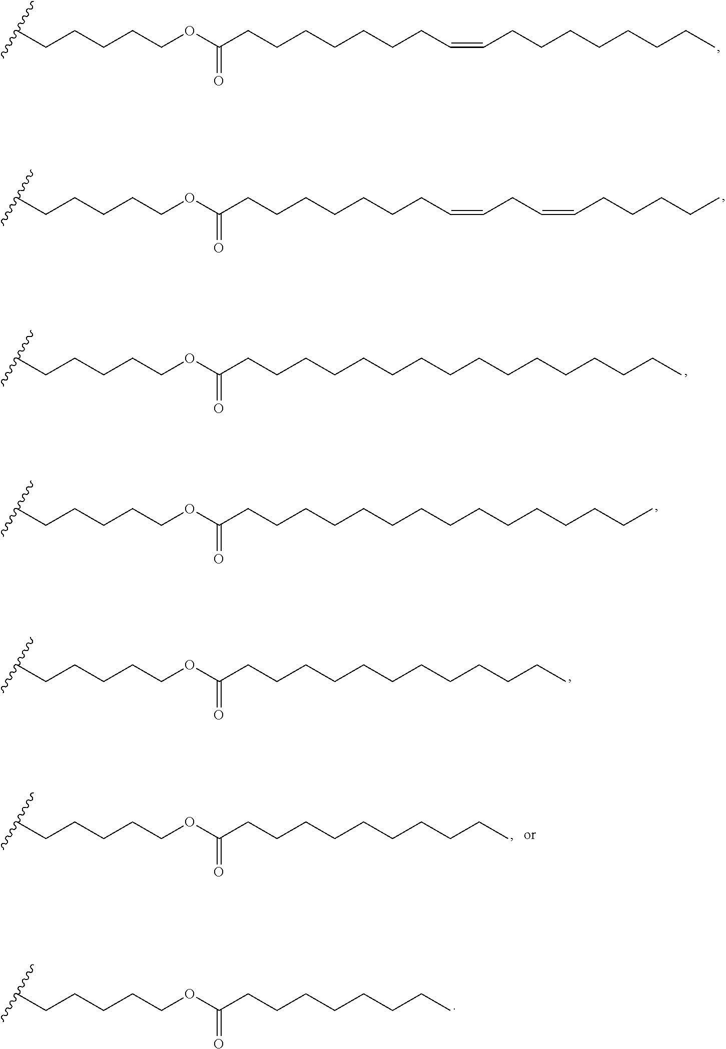

11. The compound of any one of claims 1-10, or a pharmaceutically acceptable salt, co-crystal, tautomer, stereoisomer, solvate, hydrate, polymorph, or isotopically enriched derivative thereof, wherein X² is substituted or unsubstituted C₈-C₁₀₀ aliphatic.

12. The compound of any one of claims 1-11, or a pharmaceutically acceptable salt, co-crystal, tautomer, stereoisomer, solvate, hydrate, polymorph, or isotopically enriched derivative thereof, wherein X² is substituted or unsubstituted C₁₂-C₂₀ aliphatic.

13. The compound of any one of claims 1-11, or a pharmaceutically acceptable salt, co-crystal, tautomer, stereoisomer, solvate, hydrate, polymorph, or isotopically enriched derivative thereof, wherein X² is unsubstituted C₈-C₁₀₀ aliphatic.

14. The compound of any one of claims 1-12, or a pharmaceutically acceptable salt, co-crystal, tautomer, stereoisomer, solvate, hydrate, polymorph, or isotopically enriched derivative thereof, wherein X² is substituted or unsubstituted C₁₂-C₂₀ aliphatic.

15. The compound of any one of claims 1-14, or a pharmaceutically acceptable salt, co-crystal, tautomer, stereoisomer, solvate, hydrate, polymorph, or isotopically enriched derivative thereof, wherein X² is of the formula:

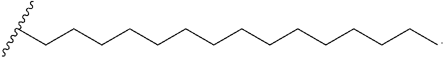 [SMILES: *CCCCCCCCCCCCCCC]

16. The compound of any one of claims 1-14, or a pharmaceutically acceptable salt, co-crystal, tautomer, stereoisomer, solvate, hydrate, polymorph, or isotopically enriched derivative thereof, wherein X² is of the formula:

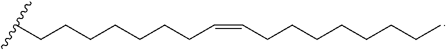 [SMILES: *CCCCCCC/C=C\CCCCCCCC]

17. The compound of any one of claims 1-14, or a pharmaceutically acceptable salt, co-crystal, tautomer, stereoisomer, solvate, hydrate, polymorph, or isotopically enriched derivative thereof, wherein X² is of the formula:

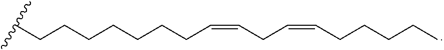 [SMILES: *CCCCCCC/C=C\C/C=C\CCCCC]

18. The compound of any one of claims 1-10, or a pharmaceutically acceptable salt, co-crystal, tautomer, stereoisomer, solvate, hydrate, polymorph, or isotopically enriched derivative thereof, wherein X² is substituted or unsubstituted C₈-C₁₀₀ heteroaliphatic.

19. The compound of any one of claims 1-10, or 18, or a pharmaceutically acceptable salt, co-crystal, tautomer, stereoisomer, solvate, hydrate, polymorph, or isotopically enriched derivative thereof, wherein X² is substituted or unsubstituted C₅-C₂₄ heteroaliphatic.

20. The compound of any one of claims 1-10, or 18-19, or a pharmaceutically acceptable salt, co-crystal, tautomer, stereoisomer, solvate, hydrate, polymorph, or isotopically enriched derivative thereof, wherein X² is of the formula:

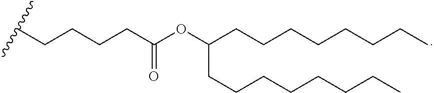 [SMILES: *CCCCC(=O)OC(CCCCCCCC)CCCCCCCC]

21. The compound of any one of claims 1-10, or 18-19, or a pharmaceutically acceptable salt, co-crystal, tautomer, stereoisomer, solvate, hydrate, polymorph, or isotopically enriched derivative thereof, wherein X² is of the formula:

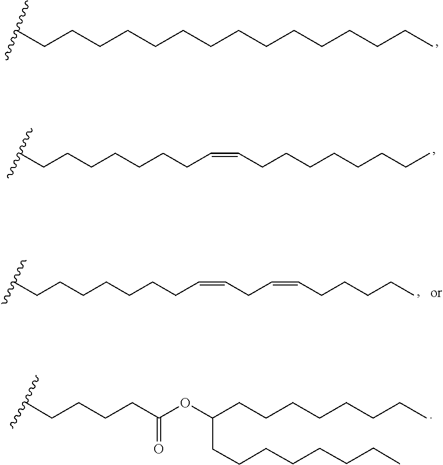 [SMILES: *CCCCCCCCCCCCCCC ; *CCCCCCC/C=C\CCCCCCCC ; *CCCCCCC/C=C\C/C=C\CCCCC ; *CCCCC(=O)OC(CCCCCCCC)CCCCCCCC]

22. The compound of any one of claims 1-21, or a pharmaceutically acceptable salt, co-crystal, tautomer, stereoisomer, solvate, hydrate, polymorph, or isotopically enriched derivative thereof, wherein R¹ is substituted or unsubstituted alkyl.

23. The compound of any one of claims 1-22, or a pharmaceutically acceptable salt, co-crystal, tautomer, stereoisomer, solvate, hydrate, polymorph, or isotopically enriched derivative thereof, wherein R¹ is substituted or unsubstituted C₁-C₆ alkyl.

24. The compound of any one of claims 1-23, or a pharmaceutically acceptable salt, co-crystal, tautomer, stereoisomer, solvate, hydrate, polymorph, or isotopically enriched derivative thereof, wherein R¹ is of the formula: 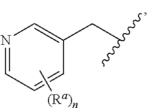
wherein R^{a} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; and
n is 1, 2, 3, or 4.

25. The compound of any one of claims 1-23, or a pharmaceutically acceptable salt, co-crystal, tautomer, stereoisomer, solvate, hydrate, polymorph, or isotopically enriched derivative thereof, wherein R¹ is substituted C₂-C₃ alkyl.

26. The compound of any one of claims 1-23, or 25, or a pharmaceutically acceptable salt, co-crystal, tautomer, stereoisomer, solvate, hydrate, polymorph, or isotopically enriched derivative thereof, wherein R¹ is of the formula: 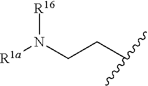
wherein:
R^{1a} and R^{1b} are each independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or R^{1a} and R^{1b} are joined together to form an optionally substituted heterocyclic ring.

27. The compound of claim 26, or a pharmaceutically acceptable salt, co-crystal, tautomer, stereoisomer, solvate, hydrate, polymorph, or isotopically enriched derivative thereof, wherein R^{1a} and R^{1b} are each independently optionally substituted C₁-C₆ alkyl.

28. The compound of claim 26 or 27, or a pharmaceutically acceptable salt, co-crystal, tautomer, stereoisomer, solvate, hydrate, polymorph, or isotopically enriched derivative thereof, wherein R^{1a} and R^{1b} are substituted or unsubstituted methyl.

29. The compound of any one of claims 1-28, or a pharmaceutically acceptable salt, co-crystal, tautomer, stereoisomer, solvate, hydrate, polymorph, or isotopically enriched derivative thereof, wherein R¹ is of the formula:

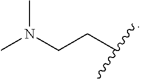 [SMILES: *CCN(C)C]

30. The compound of any one of claims 1-27, or a pharmaceutically acceptable salt, co-crystal, tautomer, stereoisomer, solvate, hydrate, polymorph, or isotopically enriched derivative thereof, wherein R¹ is of the formula:

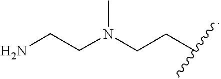 [SMILES: *CCN(C)CCN]

31. The compound of claim 26, or a pharmaceutically acceptable salt, co-crystal, tautomer, stereoisomer, solvate, hydrate, polymorph, or isotopically enriched derivative thereof, wherein R^{1a} and Rib are joined together to form an optionally substituted heterocyclic ring.

32. The compound of claim 31, or a pharmaceutically acceptable salt, co-crystal, tautomer, stereoisomer, solvate, hydrate, polymorph, or isotopically enriched derivative thereof,
wherein:
R¹ is of the formula: 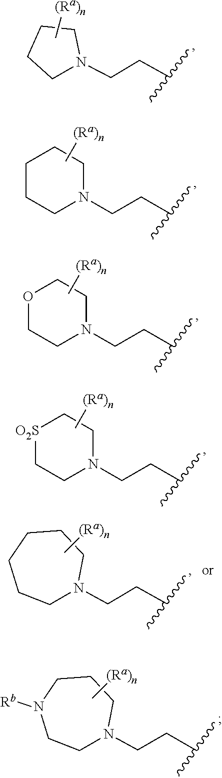
R^{a} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl;
R^{b} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or a nitrogen protecting group; and
n is 1, 2, 3, or 4.

33. The compound of claim 31 or 32, or a pharmaceutically acceptable salt, co-crystal, tautomer, stereoisomer, solvate, hydrate, polymorph, or isotopically enriched derivative thereof, wherein:
R¹ is of the formula:

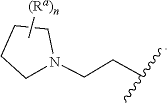

34. The compound of any one of claims 31-33, or a pharmaceutically acceptable salt, co-crystal, tautomer, stereoisomer, solvate, hydrate, polymorph, or isotopically enriched derivative thereof, wherein:
R¹ is of the formula:

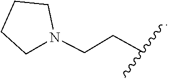 [SMILES: *CCN1CCCC1]

35. The compound of claim 31 or 32, or a pharmaceutically acceptable salt, co-crystal, tautomer, stereoisomer, solvate, hydrate, polymorph, or isotopically enriched derivative thereof, wherein:
R¹ is of the formula:

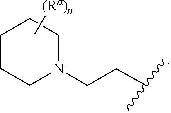

36. The compound of any one of claims 31-32, or 35, or a pharmaceutically acceptable salt, co-crystal, tautomer, stereoisomer, solvate, hydrate, polymorph, or isotopically enriched derivative thereof, wherein:
R¹ is of the formula:

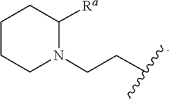

37. The compound of any one of claims 31-32, or 35-36, or a pharmaceutically acceptable salt, co-crystal, tautomer, stereoisomer, solvate, hydrate, polymorph, or isotopically enriched derivative thereof, wherein:
R¹ is of the formula:

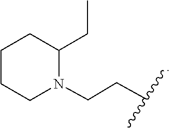 [SMILES: *CCN1CCCCC1CC]

38. The compound of claim 31 or 32, or a pharmaceutically acceptable salt, co-crystal, tautomer, stereoisomer, solvate, hydrate, polymorph, or isotopically enriched derivative thereof, wherein:
R¹ is of the formula:

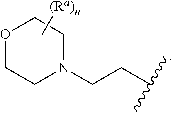

39. The compound of any one of claims 31-32, or 38, or a pharmaceutically acceptable salt, co-crystal, tautomer, stereoisomer, solvate, hydrate, polymorph, or isotopically enriched derivative thereof, wherein:
R¹ is of the formula:

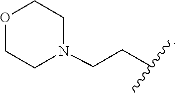 [SMILES: *CCN1CCOCC1]

40. The compound of claim 31 or 32, or a pharmaceutically acceptable salt, co-crystal, tautomer, stereoisomer, solvate, hydrate, polymorph, or isotopically enriched derivative thereof, wherein:
R¹ is of the formula:

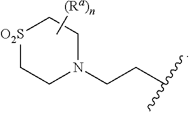

41. The compound of any one of claims 31-32, or 40, or a pharmaceutically acceptable salt, co-crystal, tautomer, stereoisomer, solvate, hydrate, polymorph, or isotopically enriched derivative thereof, wherein:
R¹ is of the formula:

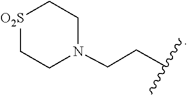 [SMILES: *CCN1CCS(=O)(=O)CC1]

42. The compound of claim 31 or 32, or a pharmaceutically acceptable salt, co-crystal, tautomer, stereoisomer, solvate, hydrate, polymorph, or isotopically enriched derivative thereof, wherein:
R¹ is of the formula:

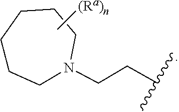

43. The compound of any one of claims 31-32, or 42, or a pharmaceutically acceptable salt, co-crystal, tautomer, stereoisomer, solvate, hydrate, polymorph, or isotopically enriched derivative thereof, wherein:
R¹ is of the formula:

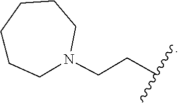 [SMILES: *CCN1CCCCCC1]

44. The compound of claim 31 or 32, or a pharmaceutically acceptable salt, co-crystal, tautomer, stereoisomer, solvate, hydrate, polymorph, or isotopically enriched derivative thereof, wherein:
R¹ is of the formula:

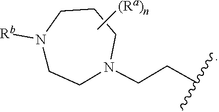

45. The compound of any one of claims 31, 32, or 44, or a pharmaceutically acceptable salt, co-crystal, tautomer, stereoisomer, solvate, hydrate, polymorph, or isotopically enriched derivative thereof, wherein:
R¹ is of the formula:

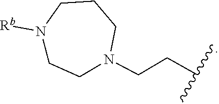

46. The compound of any one of claims 31, 32, or 44-45, or a pharmaceutically acceptable salt, co-crystal, tautomer, stereoisomer, solvate, hydrate, polymorph, or isotopically enriched derivative thereof, wherein:
R¹ is of the formula:

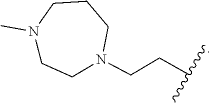 [SMILES: *CCN1CCCN(C)CC1]

47. The compound of claim 26, or a pharmaceutically acceptable salt, co-crystal, tautomer, stereoisomer, solvate, hydrate, polymorph, or isotopically enriched derivative thereof, wherein R^{1a} and R^{1b} are joined together to form an optionally substituted heteroaryl ring.

48. The compound of claim 47, or a pharmaceutically acceptable salt, co-crystal, tautomer, stereoisomer, solvate, hydrate, polymorph, or isotopically enriched derivative thereof, wherein R¹ is of the formula:

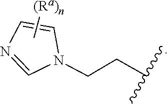

49. The compound of claim 47 or 48, or a pharmaceutically acceptable salt, co-crystal, tautomer, stereoisomer, solvate, hydrate, polymorph, or isotopically enriched derivative thereof, wherein R¹ is of the formula:

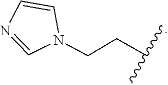 [SMILES: *CCn1ccnc1]

50. The compound of any one of claims 1-23, or 25, or a pharmaceutically acceptable salt, co-crystal, tautomer, stereoisomer, solvate, hydrate, polymorph, or isotopically enriched derivative thereof, wherein R¹ is of the formula: 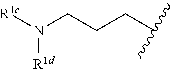
wherein:
R^{1c} and R^{1d} are each independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or R^{1a} and R^{1b} are joined together to form an optionally substituted heterocyclic ring.

51. The compound of claim 50, or a pharmaceutically acceptable salt, co-crystal, tautomer, stereoisomer, solvate, hydrate, polymorph, or isotopically enriched derivative thereof, wherein R^{1c} and R^{1d} are each independently optionally substituted C₁-C₆ alkyl.

52. The compound of claim 50 or 51, or a pharmaceutically acceptable salt, co-crystal, tautomer, stereoisomer, solvate, hydrate, polymorph, or isotopically enriched derivative thereof, wherein R^{1c} and R^{1d} are substituted or unsubstituted methyl.

53. The compound of any one of claims 1-23, or 50-52, or a pharmaceutically acceptable salt, co-crystal, tautomer, stereoisomer, solvate, hydrate, polymorph, or isotopically enriched derivative thereof, wherein R¹ is of the formula:

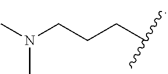 [SMILES: *CCCN(C)C]

54. The compound of any one of claims 1-23, or 50-51, or a pharmaceutically acceptable salt, co-crystal, tautomer, stereoisomer, solvate, hydrate, polymorph, or isotopically enriched derivative thereof, wherein R¹ is of the formula

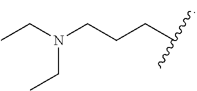 [SMILES: *CCCN(CC)CC]

55. The compound of any one of claims 1-23, or 50-51, or a pharmaceutically acceptable salt, co-crystal, tautomer, stereoisomer, solvate, hydrate, polymorph, or isotopically enriched derivative thereof, wherein R¹ is of the formula:

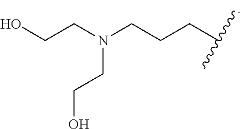 [SMILES: *CCCN(CCO)CCO]

56. The compound of any one of claims 1-23, or 50-51, or a pharmaceutically acceptable salt, co-crystal, tautomer, stereoisomer, solvate, hydrate, polymorph, or isotopically enriched derivative thereof, wherein R¹ is of the formula:

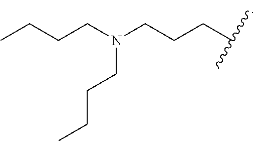 [SMILES: *CCCN(CCCC)CCCC]

57. The compound of claim 50, or a pharmaceutically acceptable salt, co-crystal, tautomer, stereoisomer, solvate, hydrate, polymorph, or isotopically enriched derivative thereof, wherein R^{1c} and R^{1d} are joined together to form an optionally substituted heterocyclic ring.

58. The compound of claim 57, or a pharmaceutically acceptable salt, co-crystal, tautomer, stereoisomer, solvate, hydrate, polymorph, or isotopically enriched derivative thereof,
wherein:
R¹ is of the formula: 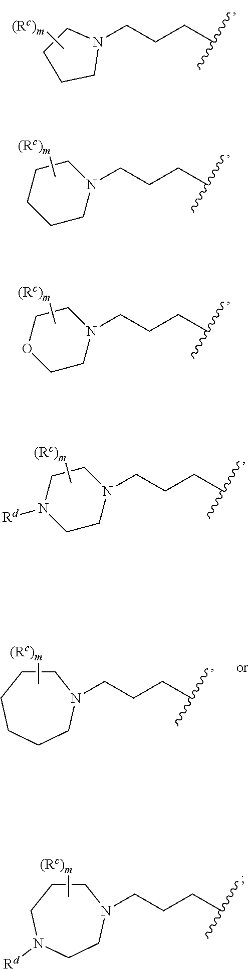
R^{c} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl;
R^{d} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or a nitrogen protecting group; and
m is 1, 2, 3, or 4.

59. The compound of claim 57 or 58, or a pharmaceutically acceptable salt, co-crystal, tautomer, stereoisomer, solvate, hydrate, polymorph, or isotopically enriched derivative thereof, wherein:
R¹ is of the formula:

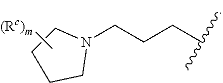

60. The compound of any one of claims 57-59, or a pharmaceutically acceptable salt, co-crystal, tautomer, stereoisomer, solvate, hydrate, polymorph, or isotopically enriched derivative thereof, wherein:
R¹ is of the formula:

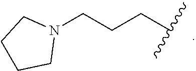 [SMILES: *CCCN1CCCC1]

61. The compound of claim 57 or 58, or a pharmaceutically acceptable salt, co-crystal, tautomer, stereoisomer, solvate, hydrate, polymorph, or isotopically enriched derivative thereof, wherein:
R¹ is of the formula:

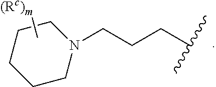

62. The compound of any one of claims 57-58, or 61, or a pharmaceutically acceptable salt, co-crystal, tautomer, stereoisomer, solvate, hydrate, polymorph, or isotopically enriched derivative thereof, wherein:
R¹ is of the formula:

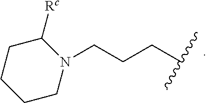

63. The compound of any one of claims 57-58, or 61-62, or a pharmaceutically acceptable salt, co-crystal, tautomer, stereoisomer, solvate, hydrate, polymorph, or isotopically enriched derivative thereof, wherein:
R¹ is of the formula:

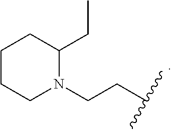 [SMILES: *CCN1CCCCC1CC]

64. The compound of claim 57 or 58, or a pharmaceutically acceptable salt, co-crystal, tautomer, stereoisomer, solvate, hydrate, polymorph, or isotopically enriched derivative thereof, wherein:
R¹ is of the formula:

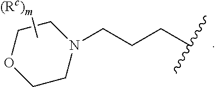

65. The compound of any one of claims 57-58, or 61, or a pharmaceutically acceptable salt, co-crystal, tautomer, stereoisomer, solvate, hydrate, polymorph, or isotopically enriched derivative thereof, wherein:
R¹ is of the formula:

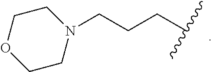 [SMILES: *CCCN1CCOCC1]

66. The compound of claim 57 or 58, or a pharmaceutically acceptable salt, co-crystal, tautomer, stereoisomer, solvate, hydrate, polymorph, or isotopically enriched derivative thereof, wherein:
R¹ is of the formula:

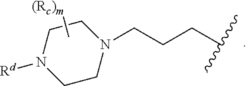

67. The compound of any one of claims 57-58, or 66, or a pharmaceutically acceptable salt, co-crystal, tautomer, stereoisomer, solvate, hydrate, polymorph, or isotopically enriched derivative thereof, wherein:
R¹ is of the formula:

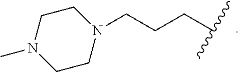 [SMILES: *CCCN1CCN(C)CC1]

68. The compound of any one of claims 57-58, or 66, or a pharmaceutically acceptable salt, co-crystal, tautomer, stereoisomer, solvate, hydrate, polymorph, or isotopically enriched derivative thereof, wherein:
R¹ is of the formula:

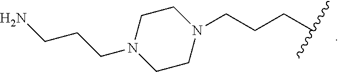 [SMILES: *CCCN1CCN(CCCN)CC1]

69. The compound of claim 47 or 48, or a pharmaceutically acceptable salt, co-crystal, tautomer, stereoisomer, solvate, hydrate, polymorph, or isotopically enriched derivative thereof, wherein:
R¹ is of the formula:

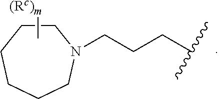

70. The compound of any one of claims 47-48, or 69, or a pharmaceutically acceptable salt, co-crystal, tautomer, stereoisomer, solvate, hydrate, polymorph, or isotopically enriched derivative thereof, wherein:
R¹ is of the formula:

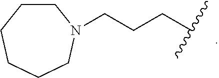 [SMILES: *CCCN1CCCCCC1]

71. The compound of claim 50 or 51, or a pharmaceutically acceptable salt, co-crystal, tautomer, stereoisomer, solvate, hydrate, polymorph, or isotopically enriched derivative thereof, wherein:
R¹ is of the formula:

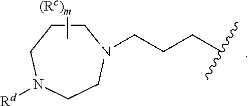

72. The compound of any one of claims 47, 48, or 71, or a pharmaceutically acceptable salt, co-crystal, tautomer, stereoisomer, solvate, hydrate, polymorph, or isotopically enriched derivative thereof, wherein:
R¹ is of the formula:

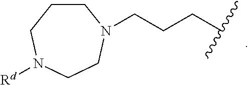

73. The compound of any one of claims 47, 48, or 71-72, or a pharmaceutically acceptable salt, co-crystal, tautomer, stereoisomer, solvate, hydrate, polymorph, or isotopically enriched derivative thereof, wherein:
R¹ is of the formula:

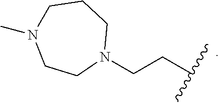 [SMILES: *CCN1CCCN(C)CC1]

74. The compound of any one of claims 1-21, or a pharmaceutically acceptable salt, co-crystal, tautomer, stereoisomer, solvate, hydrate, polymorph, or isotopically enriched derivative thereof, wherein R¹ is substituted or unsubstituted heterocyclyl.

75. The compound of any one of claims 1-21, or 74, or a pharmaceutically acceptable salt, co-crystal, tautomer, stereoisomer, solvate, hydrate, polymorph, or isotopically enriched derivative thereof, wherein R¹ is of the formula: 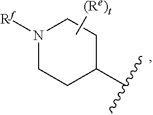
wherein:
R^{e} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl;
R^{f} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or a nitrogen protecting group; and
t is 1, 2, 3, or 4.

76. The compound of claim 75, or a pharmaceutically acceptable salt, co-crystal, tautomer, stereoisomer, solvate, hydrate, polymorph, or isotopically enriched derivative thereof, wherein R¹ is of the formula:

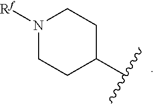

77. The compound of claim 75 or 76, or a pharmaceutically acceptable salt, co-crystal, tautomer, stereoisomer, solvate, hydrate, polymorph, or isotopically enriched derivative thereof, wherein R¹ is of the formula:

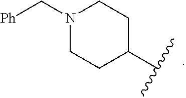 [SMILES: *C1CCN(Cc2ccccc2)CC1]

78. The compound of any one of claims 1-77, or a pharmaceutically acceptable salt, co-crystal, tautomer, stereoisomer, solvate, hydrate, polymorph, or isotopically enriched derivative thereof, wherein R is substituted or unsubstituted alkyl.

79. The compound of any one of claims 1-78, or a pharmaceutically acceptable salt, co-crystal, tautomer, stereoisomer, solvate, hydrate, polymorph, or isotopically enriched derivative thereof, wherein R is unsubstituted alkyl.

80. The compound of any one of claims 1-79, or a pharmaceutically acceptable salt, co-crystal, tautomer, stereoisomer, solvate, hydrate, polymorph, or isotopically enriched derivative thereof, wherein R is substituted or unsubstituted C₁-C₆ alkyl.

81. The compound of any one of claims 1-80, or a pharmaceutically acceptable salt, co-crystal, tautomer, stereoisomer, solvate, hydrate, polymorph, or isotopically enriched derivative thereof, wherein R is substituted or unsubstituted C₁-C₆ alkyl.

82. The compound of any one of claims 1-81, or a pharmaceutically acceptable salt, co-crystal, tautomer, stereoisomer, solvate, hydrate, polymorph, or isotopically enriched derivative thereof, wherein R is substituted or unsubstituted C₂-C₄ alkyl.

83. The compound of any one of claims 1-81, or a pharmaceutically acceptable salt, co-crystal, tautomer, stereoisomer, solvate, hydrate, polymorph, or isotopically enriched derivative thereof, wherein R is of the formula:

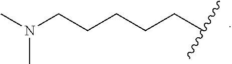 [SMILES: *CCCCCN(C)C]

84. The compound of any one of claims 1-81, or a pharmaceutically acceptable salt, co-crystal, tautomer, stereoisomer, solvate, hydrate, polymorph, or isotopically enriched derivative thereof, wherein R is of the formula:

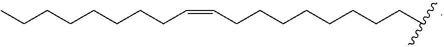 [SMILES: *CCCCCCCC/C=C\CCCCCCCC]

85. The compound of any one of claims 1-82, or a pharmaceutically acceptable salt, co-crystal, tautomer, stereoisomer, solvate, hydrate, polymorph, or isotopically enriched derivative thereof, wherein R is of the formula:

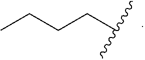 [SMILES: *CCCC]

86. The compound of any one of claims 1-82, or a pharmaceutically acceptable salt, co-crystal, tautomer, stereoisomer, solvate, hydrate, polymorph, or isotopically enriched derivative thereof, wherein R is of the formula:

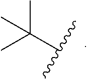 [SMILES: *C(C)(C)C]

87. The compound of any one of claims 78-79, or 81-82, or a pharmaceutically acceptable salt, co-crystal, tautomer, stereoisomer, solvate, hydrate, polymorph, or isotopically enriched derivative thereof, wherein R is of the formula:

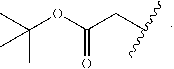 [SMILES: *CC(=O)OC(C)(C)C]

88. The compound of any one of claims 1-82, or a pharmaceutically acceptable salt, co-crystal, tautomer, stereoisomer, solvate, hydrate, polymorph, or isotopically enriched derivative thereof, wherein R is of the formula:

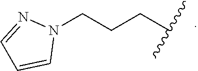 [SMILES: *CCCn1cccn1]

89. The compound of any one of claims 1-82, or a pharmaceutically acceptable salt, co-crystal, tautomer, stereoisomer, solvate, hydrate, polymorph, or isotopically enriched derivative thereof, wherein R is of the formula:

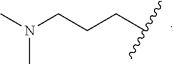 [SMILES: *CCCN(C)C]

90. The compound of any one of claims 1-82, or a pharmaceutically acceptable salt, co-crystal, tautomer, stereoisomer, solvate, hydrate, polymorph, or isotopically enriched derivative thereof, wherein R is of the formula:

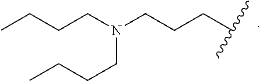 [SMILES: *CCCN(CCCC)CCCC]

91. The compound of any one of claims 1-77, or a pharmaceutically acceptable salt, co-crystal, tautomer, stereoisomer, solvate, hydrate, polymorph, or isotopically enriched derivative thereof, wherein R is substituted or unsubstituted carbocyclyl.

92. The compound of any one of claims 1-77, or 91, or a pharmaceutically acceptable salt, co-crystal, tautomer, stereoisomer, solvate, hydrate, polymorph, or isotopically enriched derivative thereof, wherein R is unsubstituted carbocyclyl.

93. The compound of any one of claims 1-77, or 91-92, or a pharmaceutically acceptable salt, co-crystal, tautomer, stereoisomer, solvate, hydrate, polymorph, or isotopically enriched derivative thereof, wherein R is substituted or unsubstituted cyclohexyl.

94. The compound of any one of claims 1-77, or 91-93, or a pharmaceutically acceptable salt, co-crystal, tautomer, stereoisomer, solvate, hydrate, polymorph, or isotopically enriched derivative thereof, wherein R is unsubstituted cyclohexyl.

95. The compound of any one of claims 1-77, or 91-93, or a pharmaceutically acceptable salt, co-crystal, tautomer, stereoisomer, solvate, hydrate, polymorph, or isotopically enriched derivative thereof, wherein R is substituted cyclohexyl.

96. The compound of any one of claims 1-77, 91-93, or 95, or a pharmaceutically acceptable salt, co-crystal, tautomer, stereoisomer, solvate, hydrate, polymorph, or isotopically enriched derivative thereof, wherein R is of the formula:

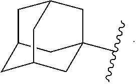 [SMILES: *C12CC3CC(CC(C3)C1)C2]

97. The compound of any one of claims 1-96, wherein the compound is of the formula: 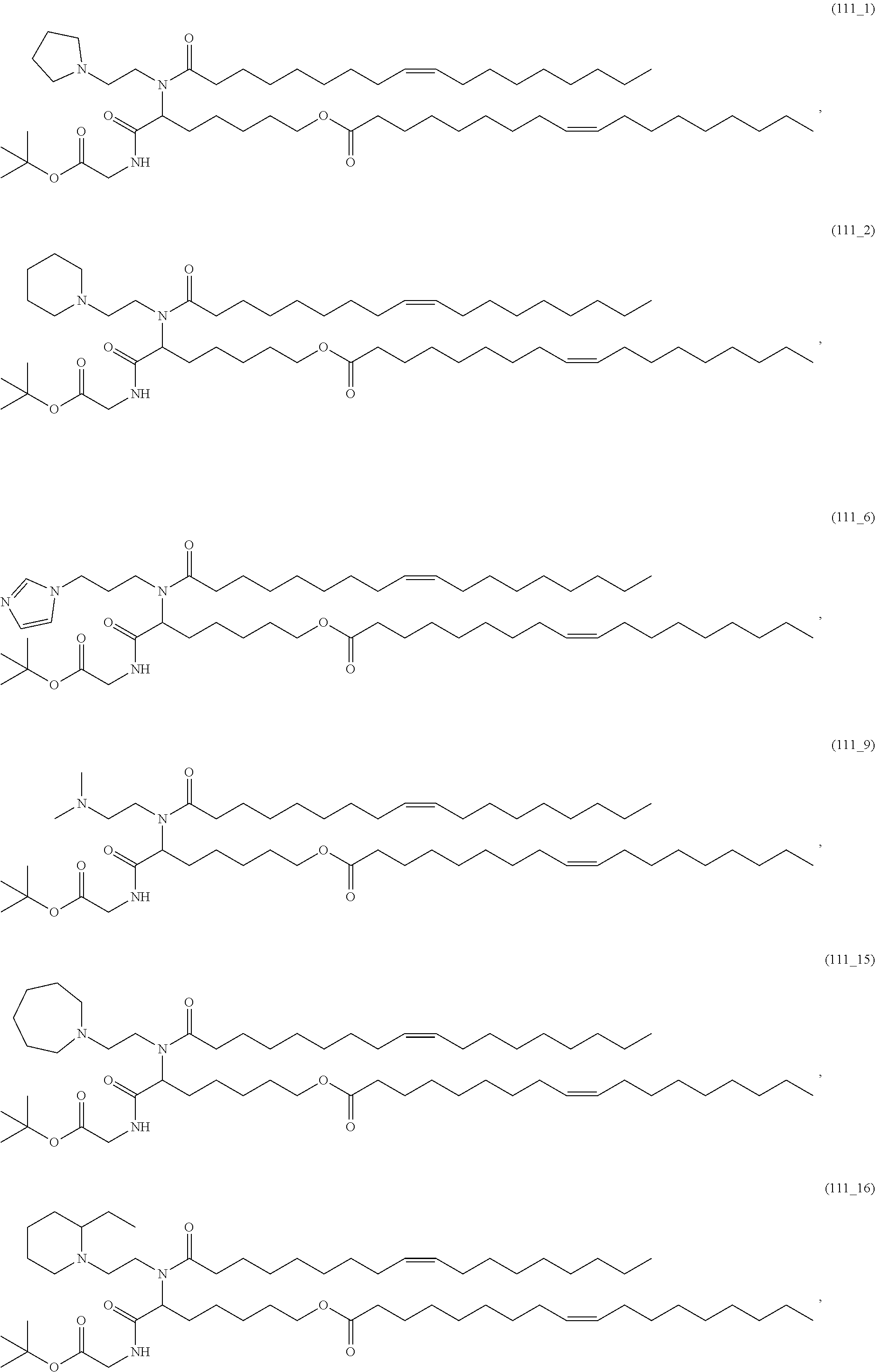 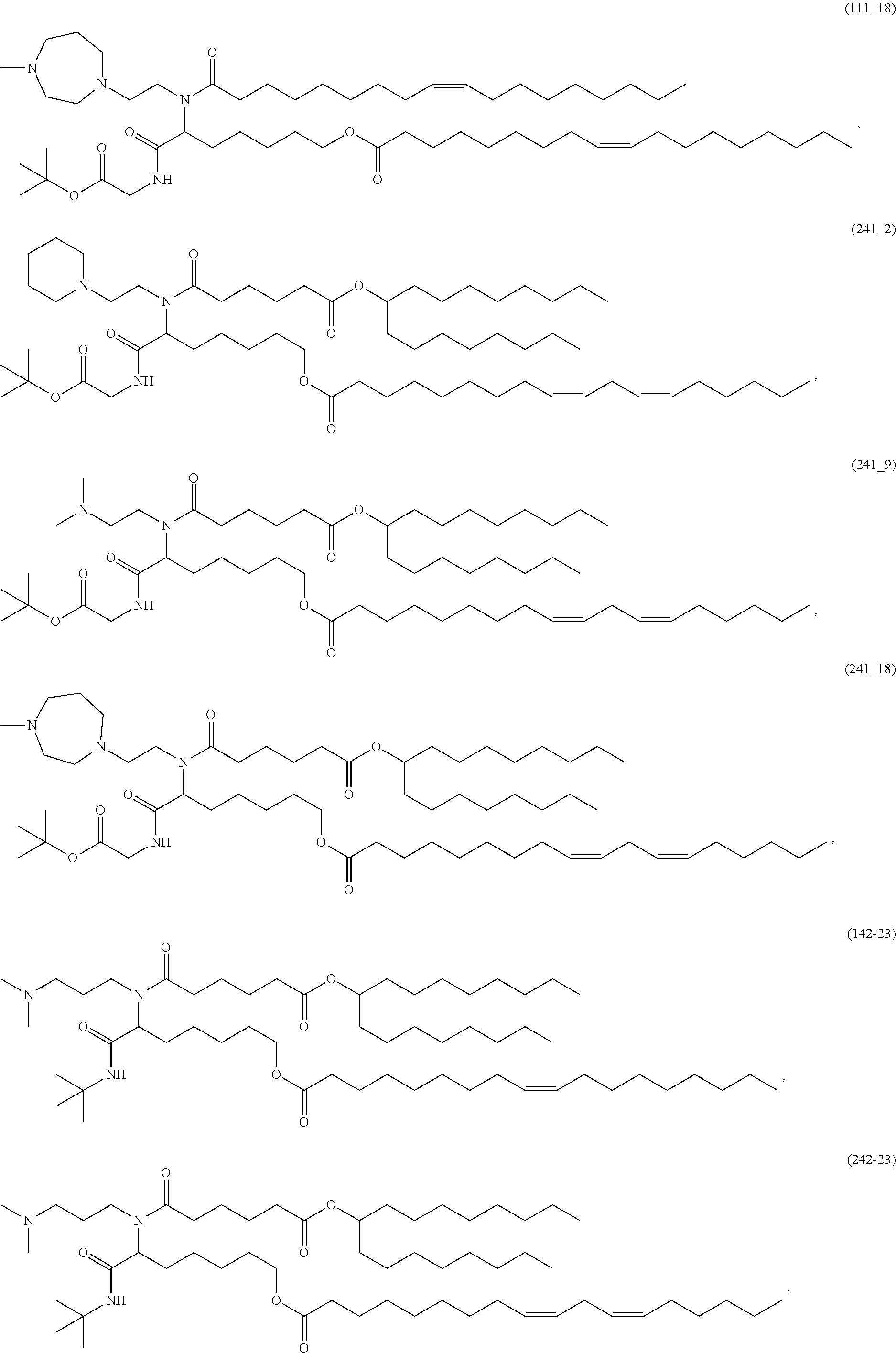 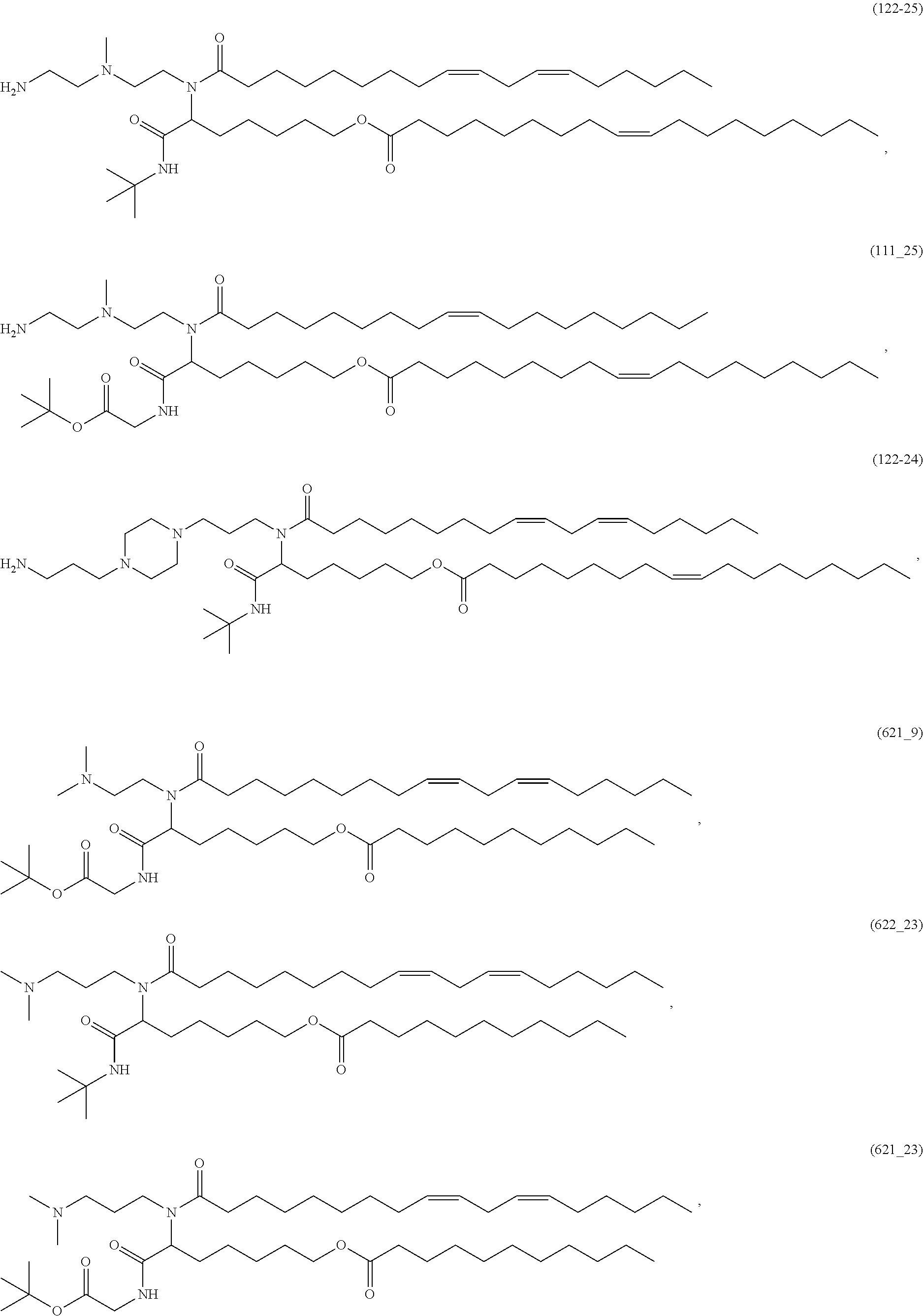 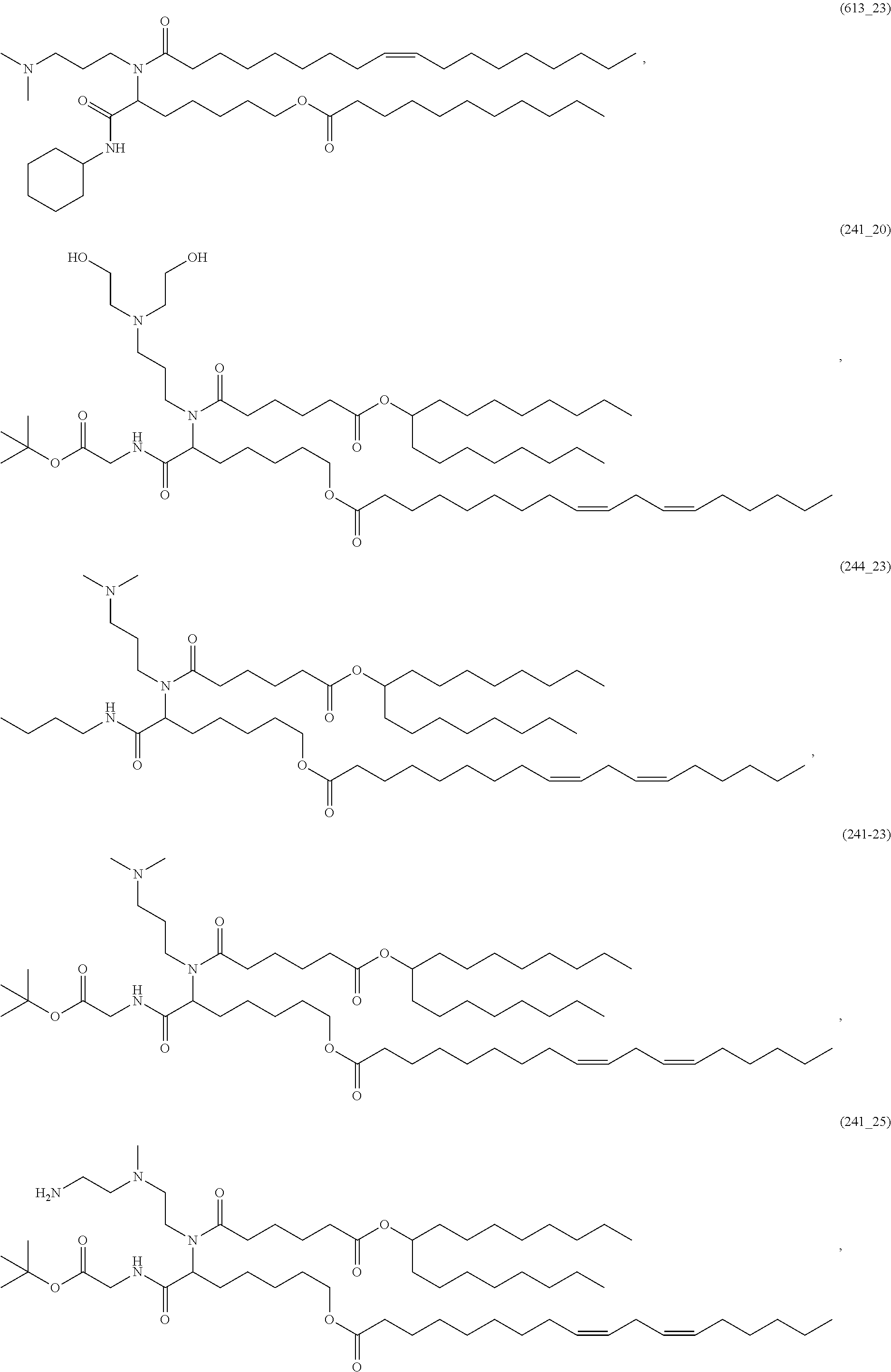 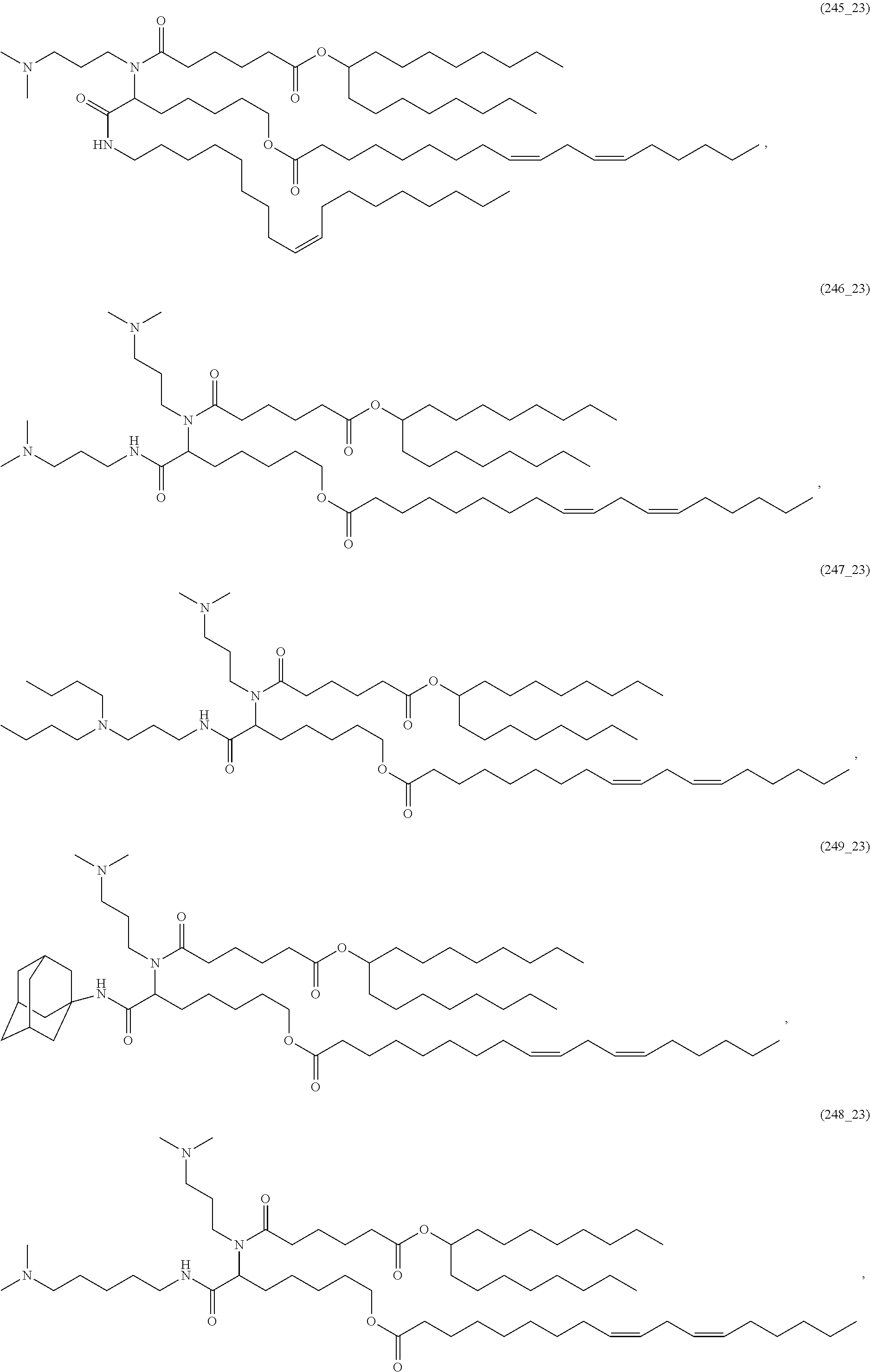 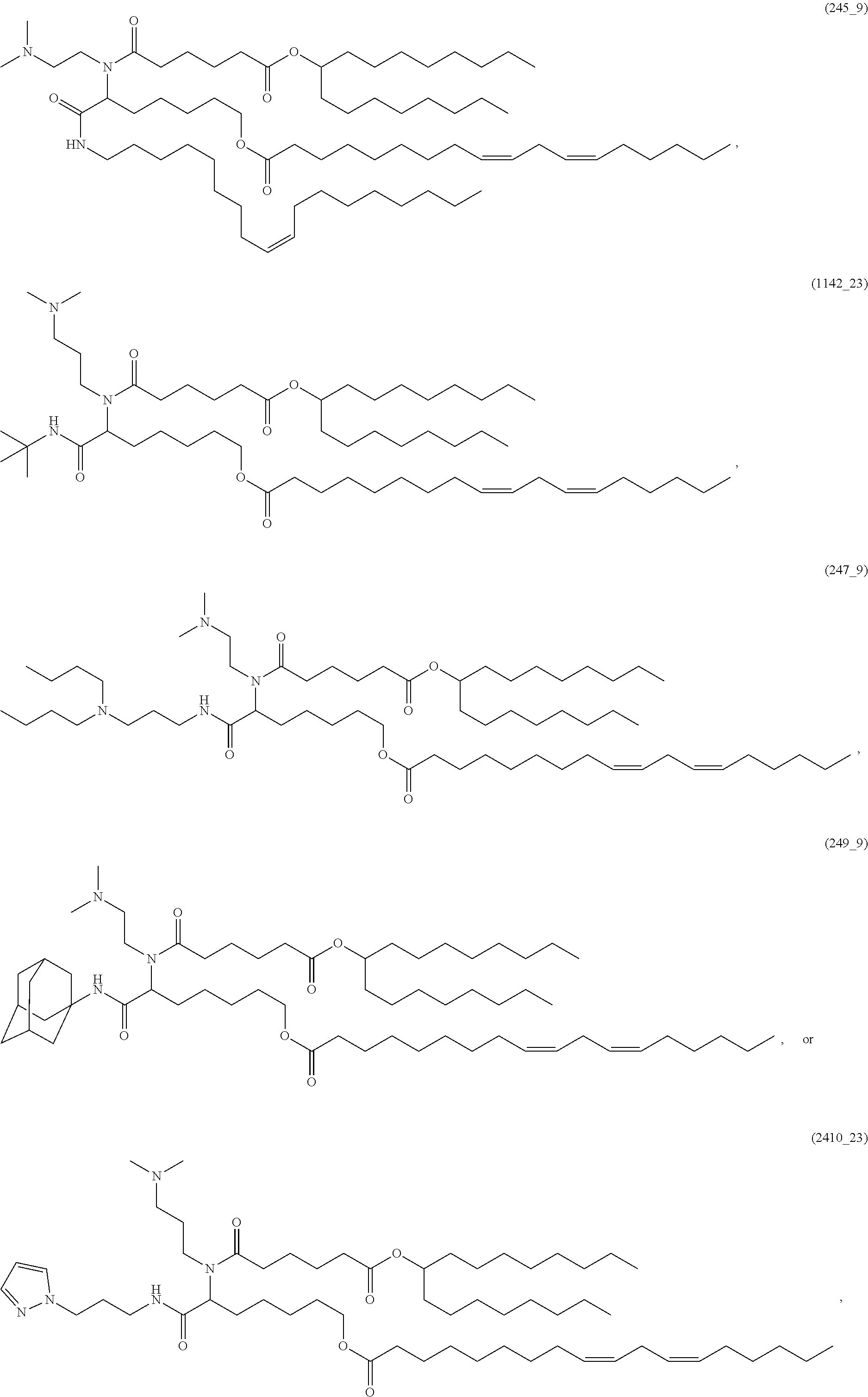
or a pharmaceutically acceptable salt, co-crystal, tautomer, stereoisomer, solvate, hydrate, polymorph, or isotopically enriched derivative thereof.

98. The compound of any one of claims 1-3, 5-14, 16, 21-23, 25, 50-53, 91-93, 95, or 96, wherein the compound is 119-23: 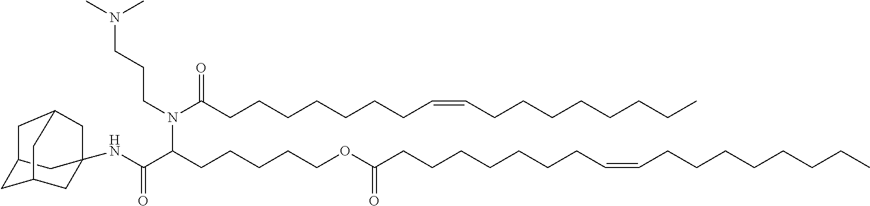 [SMILES: CCCCCCCC/C=C\CCCCCCCC(=O)OCCCCCC(C(=O)NC12CC3CC(CC(C3)C1)C2)N(CCCN(C)C)C(=O)CCCCCCC/C=C\CCCCCCCC]
or a pharmaceutically acceptable salt, co-crystal, tautomer, stereoisomer, solvate, hydrate, polymorph, or isotopically enriched derivative thereof.

99. The compound of any one of claims 1-3, 5-14, 18-23, 25, 50-53, 78-82, or 86, wherein the compound is 242-23: 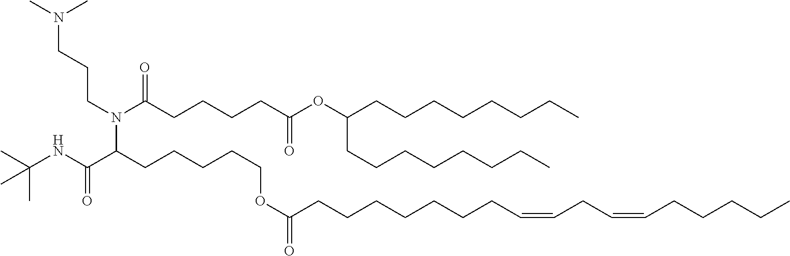 [SMILES: CCCCC/C=C\C/C=C\CCCCCCCC(=O)OCCCCCC(C(=O)NC(C)(C)C)N(CCCN(C)C)C(=O)CCCCC(=O)OC(CCCCCCCC)CCCCCCCC]
or a pharmaceutically acceptable salt, co-crystal, tautomer, stereoisomer, solvate, hydrate, polymorph, or isotopically enriched derivative thereof.

100. A composition comprising the compound, pharmaceutically acceptable salt, co-crystal, tautomer, stereoisomer, solvate, hydrate, polymorph, or isotopically enriched derivative thereof, of any one of claims 1-99; a helper lipid, a PEG-lipid, a sterol, and a polynucleotide.

101. The composition of claim 100, wherein the helper lipid is DOPE, the PEG-lipid is DMG-PEG2000, the sterol is cholesterol, and the polynucleotide is mRNA.

102. The composition of any one of claim 100 or 101, further comprising a cationic lipid.

103. The composition of claim 102, wherein the cationic lipid is DOTAP.

104. The composition of any one of claims 100-103, wherein the compound is 119-23: 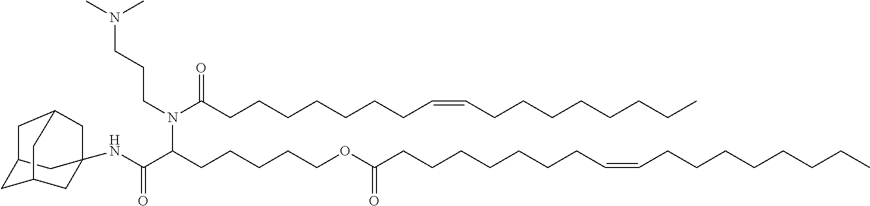 [SMILES: CCCCCCCC/C=C\CCCCCCCC(=O)OCCCCCC(C(=O)NC12CC3CC(CC(C3)C1)C2)N(CCCN(C)C)C(=O)CCCCCCC/C=C\CCCCCCCC]
or a pharmaceutically acceptable salt, co-crystal, tautomer, stereoisomer, solvate, hydrate, polymorph, or isotopically enriched derivative thereof.

105. The composition of any one of claims 100-103, wherein the compound is 242-23: 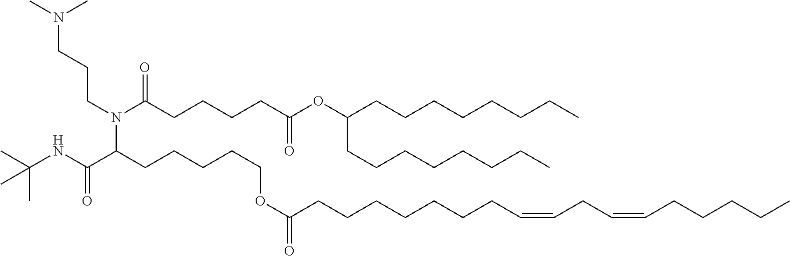 [SMILES: CCCCC/C=C\C/C=C\CCCCCCCC(=O)OCCCCCC(C(=O)NC(C)(C)C)N(CCCN(C)C)C(=O)CCCCC(=O)OC(CCCCCCCC)CCCCCCCC]
or a pharmaceutically acceptable salt, co-crystal, tautomer, stereoisomer, solvate, hydrate, polymorph, or isotopically enriched derivative thereof.

106. A composition comprising the compound, pharmaceutically acceptable salt, co-crystal, tautomer, stereoisomer, solvate, hydrate, polymorph, or isotopically enriched derivative thereof, of any one of claims 1-99; an agent; and optionally an excipient.

107. The composition of any one of claims 100-106, wherein the composition is useful for delivering the agent to a subject or cell.

108. The composition of any one of claims 100-107, wherein the composition is a pharmaceutical composition, a cosmetic composition, a nutraceutical composition, or a composition with non-medical application.

109. The composition of claim 108, wherein the composition is a pharmaceutical composition.

110. The composition of any one of claims 106-109, wherein the composition further comprises cholesterol.

111. The composition of any one of claims 106-110, wherein the composition further comprises a PEGylated lipid.

112. The composition of any one of claims 106-111, wherein the composition further comprises a phospholipid.

113. The composition of any one of claims 106-112, wherein the composition further comprises an apolipoprotein.

114. The composition of any one of claims 106-113, wherein the agent is an organic molecule, inorganic molecule, nucleic acid, protein, peptide, polynucleotide, targeting agent, an isotopically labeled chemical compound, vaccine, an immunological agent, or an agent useful in bioprocessing.

115. The composition of any one of claims 106-114, wherein the agent is a polynucleotide.

116. The composition of claim any one of claims 100, 102-105, or 115, wherein the polynucleotide is an RNA.

117. The composition of claim 116, wherein the RNA is messenger RNA (mRNA), single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA.

118. The composition of claim 116, wherein the RNA is mRNA.

119. The composition of any one of claims 100, 102-105, or 115, wherein the polynucleotide encodes a protein or a peptide.

120. The composition of claim 119, wherein the protein or peptide is an antigen.

121. The composition of any one of claims 100, 102-105, or 115, wherein the polynucleotide is a DNA.

122. The composition of claim 121, wherein the DNA is a plasmid DNA (pDNA).

123. The composition of any one of claims 100-122, wherein the composition is a vaccine.

124. The composition of any one of claims 106-123, wherein the agent and the compound are not covalently attached.

125. The composition of any one of claims 100-124, wherein the composition is in the form of a particle.

126. The composition of claim 125, wherein the particle is a nanoparticle or microparticle.

127. The composition of claim 125, wherein the particle is a micelle, liposome, or lipoplex.

128. The composition of claim 125, wherein the particle encapsulates the agent.

129. The composition of any one of claims 100-128, wherein the composition is a lyophilized solid.

130. The composition of any one of claims 100-129, wherein the composition is useful for treating or preventing a disease in a subject in need thereof.

131. The composition of claim 130, wherein the composition comprises an effective amount of the agent.

132. A method of delivering an agent to a subject or a cell, the method comprising administering to the subject or contacting the cell with a composition of any one of claims 100-131.

133. The method of claim 132, wherein the cell is a liver cell, kidney cell, spleen cell, lung cell, skin cell, brain cell, epithelial cell, or a cell of the gastrointestinal tract.

134. A method of treating or preventing a disease in a subject in need thereof, the method comprising administering to the subject a composition of any one of claims 100-131.

135. The method of claim 134, wherein the subject is a human.

136. The method of claim 134 or claim 135, wherein the disease is a genetic disease, proliferative disease, hematological disease, neurological disease, liver disease, kidney disease, spleen disease, lung disease, painful condition, psychiatric disorder, musculoskeletal disease, a metabolic disorder, inflammatory disease, or autoimmune disease.

137. A kit comprising a composition of any one of claims 100-131; and instructions for using the kit.
